(19) United States
(12) Patent Application Publication
(10) Pub. No.: US 2024/0076346 A1
(43) Pub. Date: Mar. 7, 2024

(54) TYPE I MEMBRANE PROTEINS HETERODIMERS AND METHODS OF USE THEREOF

(71) Applicants: KAHR Medical Ltd., ModiIn Makabim-ReUt (IL); Thomas Jefferson University, Philadelphia, PA (US)

(72) Inventors: Ami TAMIR, Rehovot (IL); Mark L. TYKOCINSKI, Merion Station, PA (US); Edwin BREMER, Groningen (NL)

(73) Assignees: KAHR Medical Ltd., ModiIn Makabim-ReUt (IL); Thomas Jefferson University, Philadelphia, PA (US)

(21) Appl. No.: 18/272,154

(22) PCT Filed: Jan. 13, 2021
(86) PCT No.: PCT/IL2022/050055

§ 371 Date: Jul. 13, 2023

Related U.S. Application Data
(60) Provisional application No. 63/136,687, filed on Jan. 13, 2021.
(60) Provisional application No. 63/139,331, filed on Jan. 20, 2021.

(51) Int. Cl. C07K 14/705; A61P 35/00; C07K 14/47

(52) U.S. Cl. CPC C07K 14/705; A61P 35/00; C07K 14/4703; A61K 38/00

(57) ABSTRACT

Type I membrane proteins heterodimers are provided. Accordingly, there is provided a heterodimer comprising two polypeptides selected from the group consisting of SIRPalpha, PD1, TIGIT, LILRB2 and SIGLEC10, wherein each of the two polypeptides is capable of binding a natural binding pair thereof, and wherein the heterodimer does not comprise an amino acid sequence of a type II membrane protein capable of binding a natural binding pair thereof. Also provided are nucleic acid constructs and systems encoding the heterodimer, host-cells expressing same and methods of use thereof.

### RELATED APPLICATION/S

This application claims the benefit of priority of U.S. Patent Application No. 63/136,687 filed on Jan. 13, 2021 and U.S. Patent Application No. 63/139,331 filed on Jan. 20, 2021, the contents of which are incorporated herein by reference in their entirety.

### SEQUENCE LISTING STATEMENT

The ASCII file, entitled 89962SequenceListing.txt, created on Jan. 13, 2022, comprising 303,104 bytes, submitted concurrently with the filing of this application is incorporated herein by reference.

### FIELD AND BACKGROUND OF THE INVENTION

The present invention, in some embodiments thereof, relates to type I membrane proteins heterodimers and methods of use thereof.

The interaction between cancer and the immune system is complex and multifaceted. While many cancer patients appear to develop an anti-tumor immune response, cancers also develop strategies to evade immune detection and destruction. Cancer cells can reduce the expression of tumor antigens on their surface, making it harder for the immune system to detect them; express proteins on their surface that induce immune cell inactivation; and/or induce cells in the microenvironment to release substances that suppress immune responses and promote tumor cell proliferation and survival.

Recently, immunotherapies have been developed to enhance immune responses against tumors, by stimulating specific components of the immune system or by counteracting signals produced by cancer cells that suppress immune responses. Advances in defining the mechanisms and molecules that regulate immune responses resulted in novel therapeutic targets for treating cancer. Some of these targets include: co-stimulatory and co-inhibitory molecules (e.g. CTLA4, PD1) playing a central role in the regulation of T cell immune responses, proteins that help regulate or modulate immune system activity such as interleukins and interferons, tumor antigens and components involved in activity of the innate immune system (e.g. CD47-SIRPα “don't eat-me” signal).

Additional background art includes:
International Patent Application Publication Nos. WO/2020/146423, WO201712770, WO2017152132, WO2016023001 and WO2013112986; and
U.S. Pat. Nos. 7,569,663 and 8,039,437.

### SUMMARY OF THE INVENTION

According to an aspect of some embodiments of the present invention there is provided a heterodimer comprising two polypeptides selected from the group consisting of SIRPα, PD1, TIGIT, LILRB2 and SIGLEC10, wherein each of the two polypeptides is capable of binding a natural binding pair thereof, and wherein the heterodimer does not comprise an amino acid sequence of a type II membrane protein capable of binding a natural binding pair thereof.

According to some embodiments of the invention, the heterodimer comprises a dimerizing moiety attached to the two polypeptides.

According to some embodiments of the invention, the dimerizing moiety is an Fc domain of an antibody or a fragment thereof.

According to some embodiments of the invention, the Fc domain is modified to alter it's binding to an Fc receptor, reduce an immune activating function thereof and/or improve half-life of said fusion.

According to some embodiments of the invention, the heterodimer comprises the SIRPα polypeptide and the PD1 polypeptide.

According to some embodiments of the invention, the heterodimer comprises the SIRPα polypeptide and the LILRB2 polypeptide.

According to some embodiments of the invention, the heterodimer comprises the SIRPα polypeptide and the SIGLEC10 polypeptide.

According to some embodiments of the invention, the heterodimer comprises the SIRPα polypeptide and the TIGIT polypeptide.

According to some embodiments of the invention, the heterodimer comprises the TIGIT polypeptide and the PD1 polypeptide.

According to some embodiments of the invention, the heterodimer comprises the TIGIT polypeptide and the LILRB2 polypeptide.

According to some embodiments of the invention, the heterodimer comprises the TIGIT polypeptide and the SIGLEC10 polypeptide.

According to some embodiments of the invention, the heterodimer comprises the PD1 polypeptide and the SIGLEC10 polypeptide.

According to some embodiments of the invention, the heterodimer comprises the LILRB2 polypeptide and the SIGLEC10 polypeptide.

According to some embodiments of the invention, the heterodimer comprises the PD1 polypeptide and the LILRB2 polypeptide.

According to some embodiments of the invention, each of the polypeptides is a monomer in the heterodimer

According to some embodiments of the invention, the two polypeptides are comprised in a monomer of the heterodimer.

According to an aspect of some embodiments of the present invention there is provided a composition comprising the heterodimer, wherein the heterodimer is the predominant form of the two polypeptides in the composition.

According to an aspect of some embodiments of the present invention there is provided a nucleic acid construct or system comprising at least one polynucleotide encoding the heterodimer, and a regulatory element for directing expression of the polynucleotide in a host cell.

According to an aspect of some embodiments of the present invention there is provided a host cell comprising the heterodimer or the nucleic acid construct or system.

According to an aspect of some embodiments of the present invention there is provided a method of producing a heterodimer, the method comprising introducing the nucleic acid construct or system to a host cell or culturing the cells.

According to some embodiments of the invention, the method comprising isolating the heterodimer.

According to an aspect of some embodiments of the present invention there is provided a method of treating a disease that can benefit from treatment with the heterodimer in a subject in need thereof, the method comprising administering to the subject a therapeutically effective amount of the heterodimer, the composition, the nucleic acid construct or system or the cell, thereby treating the disease in the subject.

According to an aspect of some embodiments of the present invention there is provided the heterodimer, the composition, the nucleic acid construct or system or the cell, for use in treating a disease that can benefit from treatment with the heterodimer in a subject in need thereof.

According to some embodiments of the invention, the disease that can benefit from activating immune cells.

According to some embodiments of the invention, cells associated with the disease express the natural binding pair.

According to some embodiments of the invention, the disease is cancer.

According to some embodiments of the invention, the cancer is selected from the group consisting of lymphoma, leukemia, colon carcinoma, ovarian carcinoma, lung carcinoma, head and neck carcinoma and hepatocellular carcinoma.

According to some embodiments of the invention, the cancer is non-small cell lung cancer (NSCLC) or mesothelioma.

According to an aspect of some embodiments of the present invention there is provided a method of activating immune cells, the method comprising in-vitro activating immune cells in the presence of the heterodimer, the composition, the nucleic acid construct or system or the cell.

According to some embodiments of the invention, the activating is in the presence of cells expressing the natural binding pair.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a schematic representation of non-limiting examples of possible arrangements/conformations of a heterodimer.

FIG. 1B shows schematic representations of compositions and arrangements of heterodimers contemplated by some embodiments of the invention.

FIG. 2A is a schematic representation of the SIRPα-PD1 heterodimer referred to herein as “DSP120V1” (SEQ ID NOs: 5 and 7).

FIGS. 2B-C demonstrate the predicted 3D structure of SIRPα-PD1 heterodimer DSP120V1 (SEQ ID NOs: 5 and 7). FIG. 2B is a schematic 3D model and FIG. 2C is a full atomic 3D model. SIRPα (in the ‘knob’ chain) is represented in dark grey ribbons display (lower right-hand side). PD1 (in the ‘hole’ chain) is represented in grey ribbons display (upper right-hand side). hIgG4 of the ‘knob’ sequence is represented in white ribbons in the lower right-hand side of the figure. hIgG4 of the ‘hole’ sequence is represented in grey ribbons in the upper right-hand side of the figure. ‘Spacer’/‘linker’ segments are represented in grey and white ribbons between the structural elements of SIRPα, hIgG4 and PD1. The hinge cysteine residues of the hIgG4 Fc domain (which stabilizes the complex) are represented in CPK representation.

FIG. 3A is a schematic representation of the SIRPα-LILRB2 heterodimer referred to herein as “DSP216V1” (SEQ ID NOs: 5 and 15).

FIGS. 3B-C demonstrate the predicted 3D structure of SIRPα-LILRB2 heterodimer DSP216V1 (SEQ ID NOs: 5 and 15). FIG. 3B is a schematic 3D model and FIG. 3C is a full atomic 3D model. SIRPα (in the ‘knob’ chain) is represented in dark grey ribbons display (lower right-hand side). LILRB2 (in the ‘hole’ chain) is represented in dark grey ribbons display (upper right-hand side). hIgG4 of the ‘knob’ sequence is represented in white ribbons in the lower right-hand side of the figure. hIgG4 of the ‘hole’ sequence is represented in grey ribbons in the upper right-hand side of the figure. ‘Spacer’/‘linker’ segments are represented in grey and white ribbons between the structural elements of SIRPα, hIgG4 and LILRB2. The hinge cysteine residues of the hIgG4 Fc domain (which stabilizes the complex) are represented in CPK representation.

FIG. 4A is a schematic representation of the TIGIT-SIGLEC10 heterodimer referred to herein as “DSP404V1” (SEQ ID NOs: 13 and 30).

FIGS. 4B-C demonstrate the predicted 3D structure of TIGIT-SIGLEC10 heterodimer DSP404V1 (SEQ ID NOs: 13 and 30). FIG. 4B is a schematic 3D model and FIG. 4C is a full atomic 3D model. TIGIT (in the ‘knob’ chain) is represented in grey surface display (lower right-hand side). SIGLEC10 (in the ‘hole’ chain) is represented in grey surface display (upper right-hand side). hIgG4 of the ‘knob’ sequence is represented in white surface in the lower right-hand side of the figure. hIgG4 of the ‘hole’ sequence is represented in grey surface in the upper right-hand side of the figure. ‘Spacer’/‘linker’ segments are represented in Grey and white ribbons between the structural elements of TIGIT, hIgG4 and SIGLEC10. The hinge cysteine residues of the hIgG4 Fc domain (which stabilizes the complex) are represented in CPK representation.

FIG. 5A is a schematic representation of the TIGIT-PD1 heterodimer referred to herein as “DSP502V1” (SEQ ID NOs: 13 and 7).

FIGS. 5B-C demonstrate the predicted 3D structure of TIGIT-PD1 heterodimer DSP502V1 (SEQ ID NOs: 13 and 7). FIG. 5B is a schematic 3D model and FIG. 5C is a full atomic 3D model. TIGIT (in the ‘knob’ chain) is represented in grey ribbons display (lower right-hand side). PD1 (in the ‘hole’ chain) is represented in grey ribbons display (upper right-hand side). hIgG4 of the ‘knob’ sequence is represented in white ribbons in the lower right-hand side of the figure. hIgG4 of the ‘hole’ sequence is represented in grey ribbons in the upper right-hand side of the figure. ‘Spacer’/‘linker’ segments are represented in Grey and white ribbons between the structural elements of TIGIT, hIgG4 and PD1. The hinge cysteine residues of the hIgG4 Fc domain (which stabilizes the complex) are represented in CPK representation.

FIGS. 6A-B show SDS poly acrylamide gel electrophoresis (SDS-PAGE) analysis of several produced heterodimers (see description and sequences in Table 1 hereinbelow). FIG. 6A presents SDS-PAGE images of samples of crude (non-purified)-five days-supernatant of Expi293F cells transfected with plasmids encoding the indicated heterodimers, separated under reducing (R) and/or non-reducing (NR) conditions. The control sample is a supernatant of a five days' culture of non-transfected Expi293F cells. FIG. 6B presents SDS-PAGE images of samples purified from five days-supernatants of cells transfected with constructs encoding the indicated heterodimers, using protein-A or Anion exchange chromatography as indicated.

FIGS. 7A-C present Western Blot analysis of several produced heterodimers (see description and sequences in Table 1 hereinbelow). The samples presented in the figures are crude (non-purified)-five days-supernatant of Expi293F cells transfected with plasmids encoding the indicated heterodimers. The supernatants were separated on SDS-PAGE at non-reducing (NR) or reducing (R) conditions, followed by immunoblotting with anti-PD1 (FIG. 7A), anti-SIRPα (FIG. 7B) or anti-LILRB2 (FIG. 7C) antibodies.

FIGS. 8A-B demonstrate binding of the SIRPα-PD1 heterodimer referred to herein as “DSP120” (SEQ ID NOs: 1 and 3) to CD47 and PDL1. Supernatants containing the heterodimers or control supernatant (from non-transfected Expi293F cells) were incubated in CD47 or PDL1 pre-coated 96-wells plate. Following incubation, detection was effected with anti PD-1 (For CD47 coated plate) or rabbit anti-human SIRPα antibody (for PDL1 coated plate), followed by incubation with a corresponding HRP conjugated secondary antibody. Detection was effected with a TMB substrate according to standard ELISA protocol using a Plate reader (Thermo Scientific, Multiscan FC) at 450 nm, with reference at 620. FIG. 8A shows binding of DSP120 to CD47-coated plates in a concentration dependent manner and FIG. 8B demonstrates binding of DSP120 to PDL1-coated plates in a concentration dependent manner.

FIGS. 9A-C demonstrate binding of the SIRPα-LILRB2 heterodimers referred to herein as “DSP216” (SEQ ID NOs: 1 and 11, FIG. 9A) and “DSP216V1” (SEQ ID NOs: 5 and 15, FIGS. 9B-C) to HLA-G. Supernatants containing the heterodimers or control supernatant (from non-transfected Expi293F cells) were incubated in HLA-G pre-coated 96-well plates. Binding was detected by incubation with rabbit anti-human SIRPα antibody, followed by goat anti-rabbit IgG-HRP and TMB substrate according to standard ELISA protocol using a plate reader at 450 nm, with reference at 620 nm. FIG. 9A shows binding of DSP216 to HLA-G protein coated plates in a concentration dependent manner. No binding was observed with control supernatant (control). FIGS. 9B-C show binding of crude supernatants containing DSP216V1 (FIG. 9B) or purified DSP216V1 (FIG. 9C) to HLA-G coated plates in a concentration dependent manner.

FIGS. 10A-B demonstrate binding of the PD1-TIGIT heterodimer referred to herein as “DSP502” (SEQ ID NOs: 9 and 3) to its PVR counterpart. Supernatants containing the heterodimers or control supernatant (from non-transfected Expi293F cells) (FIG. 10A) or purified protein (FIG. 10B) were incubated in a PVR pre-coated 96-wells plate. Following incubation, detection was effected with an anti-PD1 antibody followed by incubation with a corresponding HRP conjugated secondary antibody. Detection was effected with a TMB substrate according to standard ELISA protocol using a Plate reader (Thermo Scientific, Multiscan FC) at 450 nm, with reference at 620. FIGS. 10A-B demonstrate binding of DSP502 to PVR-coated plates in a concentration dependent manner.

FIGS. 11A-E demonstrate binding of DSP120 and DSP120V1 to cells expressing PDL1 or CD47, as determined by flow cytometry analysis. MFI values presented were used to create binding curves graph with a FlowJo software. FIG. 11A is a histogram demonstrating expression of PDL1 on DLD1-PDL1 overexpressing cell line. The surface expression level of PDL1 was determined by immuno-staining of DLD1 WT and PDL1 overexpressing cell lines (DLD1-PDL1) with a fluorescently labeled anti-PDL1 antibody, followed by flow cytometry analysis. FIG. 11B is a histogram demonstrating expression of the CD47 receptor on CHO-K1-CD47 HB9 clone cells. The surface expression level of CD47 was determined by immuno-staining of CHO-K1 WT and CD47 overexpressing cell lines (clone HB9) with an anti-CD47 antibody, followed by flow cytometry analysis. FIGS. 11C-D demonstrate binding of DSP120 (FIG. 11C) and DSP120V1 (FIG. 11D) to DLD1-PDL1 overexpressing cell lines compared to DLD1-WT. Binding of the heterodimer to the cell line was determined following incubation by immuno-staining of its SIRPα domain using an anti-SIRPα antibody, followed by flow cytometry analysis. FIG. 11E demonstrates binding of DSP120V1 to CHO-K1-CD47 HB9 clone cells. Binding of the heterodimer to the hCD47 overexpressing cell line was determined following incubation by immuno-staining of its IgG-Fc domain using an anti-IgG4 antibody, followed by flow cytometry analysis. CHO-K1 WT cells were used as a negative cell control for the binding assay.

FIGS. 12E-F demonstrate binding of DSP216 and DSP216V1 to cells expressing CD47 and HLA-G, as determined by flow cytometry analysis. FIGS. 12A and 12C demonstrate expression of CD47 on HT1080 (FIG. 12A) and HT1080-HLA-G (FIG. 12C) cell lines. The cell surface expression of CD47 was determined by immuno-staining of the cell lines with an anti-human-CD47 antibody and IgG1 isotype control, followed by flow cytometry analysis. FIGS. 12B and 12D demonstrate expression of HLA-G on HT1080 (FIG. 12B) and HT1080-HLA-G (FIG. 12D) cell lines. The surface expression level of HLA-G was determined by immuno-staining of the cell lines with an anti-human-HLA-G antibody and IgG2a isotype control. FIG. 12E demonstrate binding of DSP216 to the CD47 expressing cells HT1080. Binding of the heterodimer to the cell line was determined following incubation by immuno-staining of its LILRB2 domain using a LILRB2 antibody, followed by flow cytometry analysis. Percentage of cells positive for LILRB2 are presented and were used to create binding curve graphs with the GraphPad Prism software. FIG. 12F demonstrates binding of supernatant containing the heterodimer DSP216V1 to HT1080-HLA-G cell lines. The binding of the heterodimer to the cell line was determined by immuno-staining of the IgG4 backbone using an anti-IgG4 antibody, followed by flow cytometry analysis. MFI values are presented and were used to create a binding curve graph with the GraphPad Prism software.

FIG. 13 demonstrates binding of the SIGLEC10-PD1 heterodimer, referred to herein as “DSP402” (SEQ ID NOs: 24 and 3) to DLD1 WT and PDL1 overexpressing cell lines, as determined by flow cytometry analysis. Following incubation of the cells with DSP402, binding of the heterodimer to the DLD1 PDL1 overexpressing cell line was determined by immuno-staining of its PD1 domain using an anti-PD1 antibody, followed by flow cytometry analysis. DLD-1 WT cells were used as a negative cell control for the binding assay. MFI values are presented and were used to create a binding curve graph with a FlowJo software.

FIGS. 14A-G demonstrate binding of the TIGIT-PD1 heterodimer referred to herein as “DSP502” (SEQ ID NOs: 9 and 3) to cells expressing PVR and PDL1. FIGS. 14A-C demonstrate expression of PVR on DLD-1 WT (FIG. 14A), DLD-1 PDL1 (FIG. 14B) and HT1080 (FIG. 14C) cell lines. The cell surface expression of PVR was determined by immuno-staining of the cell lines with an APC labeled anti PVR antibody, followed by flow cytometry analysis. MFI values are presented. FIG. 14D demonstrates expression of PDL1 on HT1080 cells. The surface expression level of PDL1 was determined by immuno-staining of the cell line with an APC labeled anti-PDL1 antibody or isotype control, followed by flow cytometry analysis. FIGS. 14E-F demonstrate binding of DSP502 to DLD1 PDL1 (FIG. 14E) or HT1080 (FIG. 14F) cells. Binding of the heterodimer to the cell line was determined following incubation by immuno-staining of its IgG1-Fc domain using an anti-human IgG1 antibody, followed by flow cytometry analysis. Specificity of the binding to each domain of DSP502 was tested by incubating the cells with blocking Abs against PVR or PD-L1. FIG. 14G demonstrate specific binding of the TIGIT-PD1 heterodimer referred to herein as “DSP502V1” (SEQ ID NOs: 13 and 7), “DSP502V2” (SEQ ID NOs: 31 and 7), “DSP502V3” (SEQ ID NOs: 33 and 7), to PVR, demonstrated by binding to DLD-1 WT cells which express PVR and do not express PD1. Binding of the heterodimers was determined by immuno-staining of its IgG4-Fc domain using an anti-human IgG4 antibody, followed by flow cytometry analysis. MFI values are presented and were used to create binding curve graphs with a FlowJo software.

FIG. 15 shows SDS poly acrylamide gel electrophoresis (SDS-PAGE) analysis of several produced heterodimers (see description and sequences in Table 1 hereinbelow). The figure presents SDS-PAGE images of samples of crude (non-purified)-five days-supernatant of Expi293F cells transfected with plasmids encoding the indicated heterodimers, separated under reducing (R) and/or non-reducing (NR) conditions.

FIGS. 16A-F demonstrate binding of SIRPα-LILRB2 heterodimers referred to herein as “DSP216” (SEQ ID NO: 1 and 11, FIG. 16A), “DSP216V1” (SEQ ID NO: 5 and 15, FIG. 16B), “DSP216V3” (SEQ ID NO: 138 and 11, FIG. 16C), “DSP216V4” (SEQ ID NO: 140 and 15, FIG. 16D) “DSP216V5” (SEQ ID NO: 142 and 150, FIG. 16E) or “DSP216V6” (SEQ ID NO: 144 and 150, FIG. 16F) to cells overexpressing HLA-G (HT1080-HLA-G) as compared to HT1080-WT cells. Binding of the heterodimers to the cell line was determined following incubation with or without a blocking antibody, as indicated, by immuno-staining of the IgG backbone using APC conjugated anti human-IgG1 antibody, or immuno-staining of the SIRPα domain using anti-SIRPα for DSP216V1 and DSP216V4 followed by flow cytometry analysis. GMFI values are presented and were used to create binding curve graphs with the GraphPad Prism software.

FIGS. 17A-F demonstrate binding of SIRPα-LILRB2 heterodimers DSP216 (SEQ ID NO: 1 and 11, FIG. 17C), DSP216V3 (SEQ ID NO: 138 and 11, FIG. 17D), DSP216V5 (SEQ ID NO: 142 and 150, FIG. 17E) or DSP216V6 (SEQ ID NO: 144 and 150, FIG. 17F) to JEG-3 cells expressing both CD47 and HLA-G (FIGS. 17A-B). Binding of the heterodimers to the cell line was determined following incubation with or without a blocking antibody, as indicated, by immuno-staining of the IgG backbone using anti human-IgG1 antibody, followed by flow cytometry analysis. GMFI values are presented and were used to create binding curve graphs with the GraphPad Prism software.

FIGS. 18A-D demonstrate binding of SIRPα-LILRB2 heterodimers DSP216 (SEQ ID NO: 1 and 11, FIG. 18A), DSP216V1 (SEQ ID NO: 5 and 15, FIG. 18B), DSP216V3 (SEQ ID NO: 138 and 11, FIG. 18C) and DSP216V4 (SEQ ID NO: 140 and 15, FIG. 18D) to plate bound (PB) recombinant human CD47. Supernatants containing the heterodimers were incubated in CD47 pre-coated 96-well plates. Binding was detected by incubation with an anti-human IgG1- or IgG4-HRP antibody, detection with a TMB substrate, according to a standard ELISA protocol using a plate reader (Thermo Scientific, Multiscan FC) at 450 nm, with reference at 620 nm. O.D. values were used to create a binding curve graph with a GraphPad Prism software.

FIGS. 18E-H demonstrate binding of SIRPα-LILRB2 heterodimers DSP216 (SEQ ID NO: 1 and 11, FIG. 18E), DSP216V1 (SEQ ID NO: 5 and 15, FIG. 18F), DSP216V3 (SEQ ID NO: 138 and 11, FIG. 18G) and DSP216V4 (SEQ ID NO: 140 and 15, FIG. 18H) to plate bound (PB) recombinant human HLA-G. Supernatants containing the heterodimers were incubated in HLA-G pre-coated 96-well plates. Binding was detected by incubation with an anti-human IgG1- or IgG4-HRP antibody, detection with a TMB substrate, according to a standard ELISA protocol using a plate reader (Thermo Scientific, Multiscan FC) at 450 nm, with reference at 620 nm. O.D. values were used to create a binding curve graph with a GraphPad Prism software.

FIGS. 19A-I demonstrate binding of the TIGIT-PD1 heterodimers DSP502 (SEQ ID NOs: 9 and 3) and the heterodimer referred to herein as “DSP502V4” (SEQ ID NOs: 146 and 148) to cells expressing PDL1 and/or PVR, as determined by flow cytometry analysis. MFI values presented were used to create binding curve graphs with a FlowJo software. FIG. 19A demonstrates binding of DSP502 to K562 PD-L1 cells; FIG. 19B demonstrates binding of DSP502 to K562 PD-L1/PVR cells; FIG. 19C demonstrates binding of DSP502V4 to K562 PD-L1 cells; FIG. 19D demonstrates binding of DSP502V4 to K562 PD-L1/PVR cells; FIG. 19G demonstrates binding of DSP502 to K562 PVR cells; and FIG. 19H demonstrates binding of DSP502V4 to K562 PVR cells. FIGS. 19E, 19F and 191 are histograms demonstrating expression of PDL1 or PVR, as indicted, on K562 PD-L1, K562 PVR and K562 PD-L1/PVR cells. The surface expression levels of PDL1 and PVR were determined by immuno-staining of the cells with a fluorescently labeled anti-PDL1 or anti-PVR antibody, as indicated, followed by flow cytometry analysis.

FIG. 20A demonstrates binding of the TIGIT-PD1 heterodimer DSP502 (SEQ ID NOs: 9 and 3) to SKOV3 cells expressing PVR, as determined by flow cytometry analysis following incubation with or without a blocking antibody, as indicated. MFI values presented were used to create binding curve graphs with a FlowJo software.

FIG. 20B is a histogram demonstrating expression of PVR on SKOV3 cells. The surface expression level of PVR was determined by immuno-staining of the cells with a fluorescently labeled anti-PVR antibody, followed by flow cytometry analysis.

FIG. 21A demonstrates binding of the TIGIT-PD1 heterodimer DSP502 (SEQ ID NOs: 9 and 3) to Renca cells expressing mouse PDL1 and PVR, as determined by flow cytometry analysis following incubation with or without a blocking antibody, as indicated. MFI values presented were used to create binding curve graphs with a FlowJo software.

FIG. 21B shows histograms demonstrating expression of PDL-1 and PVR on Renca cells. The surface expression levels of PDL1 and PVR were determined by immuno-staining of the cells with a fluorescently a labeled anti-PDL1 or anti-PVR antibody, as indicated, followed by flow cytometry analysis.

FIG. 22A demonstrates binding of the TIGIT-PD1 heterodimer DSP502 (SEQ ID NOs: 9 and 3) to the murine cell line AB12, as determined by flow cytometry analysis. MFI values presented were used to create binding curve graphs with a FlowJo software.

FIG. 22B is a histogram demonstrating surface expression levels of PDL1 and PVR on AB12 cells. The surface expression levels of PDL1 and PVR were determined by immuno-staining of the cells with a fluorescently labeled anti-PDL1 or anti-PVR antibody, as indicated, followed by flow cytometry analysis.

FIG. 23A demonstrates binding of the TIGIT-PD1 heterodimer DSP502 (SEQ ID NOs: 9 and 3) to Jurkat NFAT-CD16 cells, as determined by flow cytometry analysis following incubation with or without a blocking antibody, as indicated. MFI values presented were used to create binding curve graphs with a FlowJo software.

FIG. 23B is a histogram demonstrating expression of CD16 on Jurkat NFAT-CD16 cells. The surface expression level of CD16 was determined by immuno-staining of the cells with a fluorescently labeled anti-CD16 antibody, as indicated, followed by flow cytometry analysis.

FIG. 24 demonstrates luciferase secretion levels from Jurkat NFAT-CD16 cells, following co-culturing with K562-WT or K562 overexpressing PDL1 and PVR cells in the presence of various concentrations of DSP502 (SEQ ID NOs: 9 and 3). The levels of Luciferase secretion were measured as a luminescence signal, produced by interaction of luciferase and added substrate (QUANTI-Luc).

FIG. 25A demonstrates simultaneous binding of the TIGIT and PD1 domains of DSP502 (SEQ ID NOs: 9 and 3) to their counterpart ligands/receptors. FIG. 25A shows binding to plate bound PDL1 followed by incubation with human CD155 (PVR)-Mouse IgG2a Fc. Detection was effected with streptavidin HRP followed by adding a TMB substrate according to a standard ELISA protocol using a Plate reader (Thermo Scientific, Multiscan FC) at 450 nm, with reference at 540 nm.

FIGS. 25B-D demonstrate doublets formation of NK cells and K562 PVR/PD-L1 cells in the presence of various concentrations of the TIGIT-PD1 heterodimer DSP502 (SEQ ID NOs: 9 and 3). FIG. 25B is a histogram demonstrating the expression level of CD16 on NK cells, determined by immuno-staining of the cells with a fluorescently labeled anti-CD16 antibody, followed by flow cytometry analysis. FIG. 25C demonstrates doublets formation in the presence of the indicated concentrations of DSP502. FIG. 25D demonstrates doublets formation in the presence of the indicated concentrations of DSP502 following incubation with a blocker antibody: Fc blocking, PVR blocking or PD-L1 blocking, as indicated. Q1 (upper left quarter in each panel) represents the K562 PVR/PD-L1 CFSE labeled cells (positive cells on Y axis); Q3 (lower righty quarter in each panel) represents NK CPD labeled cells (positive cells on X axis); and Q2 (upper right quarter in each panel) represents doublets of NK-K562 PVR/PD-L1 cells with the doublet's percentage.

FIG. 26 demonstrates the in-vivo anti-tumor effect of the TIGIT-PD1 heterodimer DSP502 (SEQ ID NOs: 9 and 3) in an A549-NSCLC xenograft model in a humanized NSG mouse, manifested by reduced tumor volume compared to a vehicle control. n=5 in each experimental group.

FIG. 27 demonstrates the in-vivo anti-tumor effect of the TIGIT-PD1 heterodimer DSP502 (SEQ ID NOs: 9 and 3) in mice bearing AB12 mesothelioma tumors, manifested by prolonged survival as compared to a vehicle control. n=5 in each experimental group.

FIG. 28 demonstrates the effect of the SIRPα-LILRB2 heterodimer DSP216 (SEQ ID NO: 1 and 11, FIG. 16A) on the phagocytosis of cancer cells by granulocytes. HT1080 or HT1080-HLA-G cells were labelled and pre-incubated with 0, 1, 2 or 5 μg/mL DSP216, then co-cultured 1:1 with granulocytes and analyzed by Flow cytometry. Shown are percentages of phagocytosis by granulocytes taken from three donors.

FIG. 29A demonstrates cytotoxic effect of the TIGIT-PD1 heterodimer DSP502 (SEQ ID NOs: 9 and 3). Shown are percentages of dead cells following co-culturing of the indicated K562 cells with NK cells, at the indicated ratios, in the presence of DSP502. Asterisks above the bars represent the statistical significance relative to an untreated co-culture.

FIG. 29B demonstrates the effect of the TIGIT-PD1 heterodimer DSP502 (SEQ ID NOs: 9 and 3) on Granzyme B secretion from NK cells following co-culturing with the indicated K562 cells. Asterisks above the bars represent the statistical significance relative to an untreated co-culture.

### DESCRIPTION OF SPECIFIC EMBODIMENTS OF THE INVENTION

The present invention, in some embodiments thereof, relates to type I membrane proteins heterodimers and methods of use thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Cancer immunotherapies aim to enhance immune responses against tumors by stimulating specific components of the immune system or by counteracting signals produced by cancer cells that suppress immune responses.

Whilst reducing specific embodiments of the present invention to practice, the present inventors have now generated heterodimers comprising extracellular portions of two type I membrane proteins selected from the group consisting of SIRPα, PD1, TIGIT, LILRB2 and SIGLEC10 (see for example Table 1 hereinbelow).

Thus, according to an aspect of the present invention, there is provided a heterodimer comprising two polypeptides selected from the group consisting of SIRPα, PD1, TIGIT, LILRB2 and SIGLEC10, wherein each of said two polypeptides is capable of binding a natural binding pair thereof, and wherein said heterodimer does not comprise an amino acid sequence of a type II membrane protein capable of binding a natural binding pair thereof.

As used herein, the term “heterodimer” refers to a non-naturally occurring dimeric protein formed by the artificial attachment of two different proteins (referred to herein as monomers).

Methods of determining dimerization, and specifically heterodimerization, are well known in the art and include, but are not limited to NATIVE-PAGE, SEC-HPLC 2D gels, gel filtration, SEC-MALS, Analytical ultracentrifugation (AUC) Mass spectrometry (MS), capillary gel electrophoresis (CGE).

According to specific embodiments, the monomers of the heterodimer are not covalently attached.

According to other specific embodiments, the monomers of the heterodimer are covalently attached.

According to other specific embodiments, the monomers of the heterodimer are attached by a disulfide bond.

According to specific embodiments, the monomers of the heterodimer are attached by disulfide bonds.

As used herein, the terms “SIRPα polypeptide”, “PD1 polypeptide”, “TIGIT polypeptide”, “LILRB2 polypeptide” and “SIGLEC10 polypeptide” refer to the amino acid sequences, or functional homolog thereof, of SIRPα, PD1, TIGIT, LILRB2 and SIGLEC10, respectively, capable of at least binding a natural binding pair thereof, as further described hereinbelow.

As use herein, the phrase “a functional homolog” refers to a fragment, a homologue (naturally occurring or synthetically/recombinantly produced) and/or an amino acid sequence comprising conservative and non-conservative amino acid substitutions, which maintains at least the activity of the full length protein of binding its natural binding pair.

As used herein, the phrase “a natural binding pair thereof” refers to the native ligand or receptor of the recited polypeptide.

Assays for testing binding are well known in the art and include, but not limited to flow cytometry, BiaCore, bio-layer interferometry Blitz® assay, HPLC.

According to specific embodiments, the heterodimer comprises a PD1 polypeptide.

As used herein the term “PD1 (Programmed Death 1, also known as CD279)” refers to the polypeptide encoded by the PDCD1 gene (Gene ID 5133). According to specific embodiments, PD1 is human PD1. According to a specific embodiment, the PD1 refers to the human PD1, such as provided in the following GenBank Number NP_005009.

Two ligands for PD1 have been identified so far, PDL1 and PDL2 (also known as B7-DC). According to a specific embodiment, the PDL1 protein refers to the human protein, such as provided in the following GenBank Number NP_001254635 and NP_054862. According to a specific embodiment, the PDL2 protein refers to the human protein, such as provided in the following GenBank Number NP_079515.

According to specific embodiments, PD1 amino acid sequence comprises SEQ ID NO: 37.

According to specific embodiments, PD1 amino acid sequence consists of SEQ ID NO: 37.

According to specific embodiments, the PD1 polypeptide binds PD-L1 with a Kd of 1 nM-100 μM, 10-nM-10 μM, 100 nM-100 μM, 200 nM-10 μM, as determined by SPR analysis, each possibility represents a separate embodiment of the present invention.

According to specific embodiments, the PD1 polypeptide binds PDL1 with a Kd of about 270 nM as determined by SPR analysis.

According to specific embodiments, the PD1 polypeptide binds PDL1 with a Kd of about 8-9 μM as determined by SPR analysis.

According to specific embodiments, the PD1 polypeptide comprises an extracellular domain of PD1 or a functional homolog (e.g. fragment) thereof.

According to specific embodiments, the PD1 polypeptide comprises SEQ ID NO: 41, 42 or 43 or a functional homolog (e.g. fragment) thereof.

According to specific embodiments, the PD1 polypeptide comprises SEQ ID NO: 41, 42 or 43.

According to specific embodiments, PD1 amino acid sequence consists of SEQ ID NO: 41, 42 or 43.

The term “PD1 polypeptide” also encompasses functional homologues which exhibit the desired activity (i.e., binding PD-L1 and/or PD-L2). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 37, 41, 42, or 43 or any other PD1 amino acid sequence disclosed herein; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

As used herein, “identity” or “sequence identity” refers to global identity, i.e., an identity over the entire amino acid or nucleic acid sequences disclosed herein and not over portions thereof.

Sequence identity or homology can be determined using any protein or nucleic acid sequence alignment algorithm such as Blast, ClustalW, and MUSCLE.

The homolog may also refer to an ortholog, a deletion, insertion, or substitution variant, including an amino acid substitution, as further described hereinbelow.

According to specific embodiments, the PD1 polypeptide may comprise conservative and non-conservative amino acid substitutions. Such substitution are known in the art and disclosed e.g. in Maute et al. PNAS, 2015 Nov. 24; 112(47):E6506-14; Ju Yeon et al. Nature Communications 2016 volume 7, Article number: 13354 (DOI: 10.1038/ncomms13354); Zack K M et al. Structure. 2015 23(12): 2341-2348 (DOI:10.1016/j.str.2015.09.010); and US Patent Application Publication No. 2016/0039903, the contents of which are fully incorporated herein by reference.

According to specific embodiments, the mutations result in increased affinity of the PD1 polypeptide to PDL1 as compared to SEQ ID NO: 37.

According to specific embodiments, one or more amino acid mutations are located at an amino acid residue selected from: V39, L40, N41, Y43, R44, M45, S48, N49, Q50, T51, D52, K53, A56, Q63, G65, Q66, V72, H82, M83, R90, Y96, L97, A100, S102, L103, A104, P105, K106, and A107 corresponding to the PD1 amino acid sequence set forth in SEQ ID NO: 42. According to specific embodiments, one or more amino acid mutations are located at an amino acid residue selected from: V39, L40, N41, Y43, R44, M45, S48, N49, Q50, T51, D52, K53, A56, Q63, G65, Q66, C68, V72, H82, M83, R90, Y96, L97, A100, S102, L103, A104, P105, K106, and A107 corresponding to the PD1 amino acid sequence set forth in SEQ ID NO: 42.

According to specific embodiments, one or more amino acid changes are selected from the group consisting of: (1) V39H or V39R; (2) L40V or L40I; (3) N41I or N41V; (4) Y43F or Y43H; (5) R44Y or R44L; (6) M45Q, M45E, M45L, or M45D; (7) S48D, S48L, S48N, S48G, or S48V; (8) N49C, N49G, N49Y, or N49S; (9) Q50K, Q50E, or Q50H; (10) T51V, T51L, or T51A; (11) D52F, D52R, D52Y, or D52V; (12) K53T or K53L; (13) A56S or A56L; (14) Q63T, Q63I, Q63E, Q63L, or Q63P; (15) G65N, G65R, G65I, G65L, G65F, or G65V; (16) Q66P; (17) V72I; (18) H82Q; (19) M83L or M83F; (20) R90K; (21) Y96F; (22) L97Y, L97V, or L97I; (23) A100I or A100V; (24) S102T or S102A; (25) L103I, L103Y, or L103F; (26) A104S, A104H, or A104D; (27) P105A; (28) K106G, K106E, K106I, K106V, K106R, or K106T; and (29) A107P, A107I, or A107V corresponding to the PD1 amino acid sequence set forth in SEQ ID NO: 42.

According to specific embodiments, one or more amino acid changes are selected from the group consisting of: (1) V39H or V39R; (2) L40V or L40I; (3) N41I or N41V; (4) Y43F or Y43H; (5) R44Y or R44L; (6) M45Q, M45E, M45L, or M45D; (7) S48D, S48L, S48N, S48G, or S48V; (8) N49C, N49G, N49Y, or N49S; (9) Q50K, Q50E, or Q50H; (10) T51V, T51L, or T51A; (11) D52F, D52R, D52Y, or D52V; (12) K53T or K53L; (13) A56S or A56L; (14) Q63T, Q63I, Q63E, Q63L, or Q63P; (15) G65N, G65R, G65I, G65L, G65F, or G65V; (16) Q66P; (17) C68S (18), V72T; (19) H82Q; (20) M83L or M83F; (21) R90K; (22) Y96F; (23) L97Y, L97V, or L97I; (24) A100I or A100V; (25) S102T or S102A; (26) L103I, L103Y, or L103F; (27) A104S, A104H, or A104D; (28) P105A; (29) K106G, K106E, K106I, K106V, K106R, or K106T; and (30) A107P, A107I, or A107V corresponding to the PD1 amino acid sequence set forth in SEQ ID NO: 42.

According to specific embodiments, an amino acid mutation is located at an amino acid residue C93 corresponding to the PD1 amino acid sequence set forth in SEQ ID NO: 37 (e.g. equivalent to an amino acid residue C68 corresponding to the PD1 amino acid sequence set forth in SEQ ID NO: 42).

Thus, according to specific embodiments, the PD1 polypeptide comprises SEQ ID NO: 39 or a functional homolog (e.g. fragment) thereof.

According to specific embodiments, the PD1 polypeptide comprises SEQ ID NO: 39.

According to specific embodiments, PD1 amino acid sequence consists of SEQ ID NO: 39.

As used herein, the phrase “corresponding to PD1 amino acid sequence as set forth in SEQ ID NO: 37”, “corresponding to SEQ ID NO: 37”, “corresponding to PD1 amino acid sequence as set forth in SEQ ID NO: 42” or “corresponding to SEQ ID NO: 42”, intends to include the corresponding amino acid residue relative to any other PD1 amino acid sequence.

Additional description on conservative amino acid and non-conservative amino acid substitutions is further provided hereinabove and below.

The PD1 polypeptide of some embodiments of the present invention is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 39, 41, 42, 43, 45, 47, 49, 53, 55, 57, 59, 61, 63, 65, 67, 69, 71, 73, 75, 77, 79 or 81; or at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same, each possibility represents a separate embodiment of the present invention.

According to specific embodiments, the PD1 polypeptide does not comprise any of amino acid segments P1-L5 and/or F146-V150 corresponding to SEQ ID NO: 43.

According to specific embodiments, the PD1 polypeptide does not comprise any of amino acid residues P1-L5 and/or F146-V150 corresponding to SEQ ID NO: 43.

According to specific embodiments, PD1 polypeptide comprises 100-288 amino acids, 100-200 amino acids, 120-180 amino acids, 120-160, 130-170 amino acids, 130-160, 130-150, 140-160 amino acids, 145-155 amino acids, 123-166 amino acids, 138-145 amino acids, 123-148 amino acids, 126-148 amino acids, 123-140 amino acids, 126-140 amino acids, 127-140 amino acids, 130-140 amino acids, each possibility represents a separate embodiment of the present invention.

According to specific embodiments, the PD1 polypeptide comprises an amino acid sequence selected from the group consisting of SEQ ID NO: 39, 41, 42, 43, 45, 47, 49, 53, 55, 57, 59, 61, 63, 65, 67, 69, 71, 73, 75, 77, 79 and 81.

According to specific embodiments, the PD1 polypeptide consists of an amino acid sequence selected from the group consisting of SEQ ID NO: 39, 41, 42, 43, 45, 47, 49, 53, 55, 57, 59, 61, 63, 65, 67, 69, 71, 73, 75, 77, 79 and 81.

According to specific embodiments, the PD1 polypeptide comprises SEQ ID NO: 49 or a functional homolog (e.g. fragment) thereof.

According to specific embodiments, the PD1 polypeptide comprises SEQ ID NO: 49.

According to specific embodiments, the PD1 polypeptide consists of SEQ ID NO: 49.

According to specific embodiments, the nucleic acid sequence encoding the PD1 polypeptide has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 38, 40, 44, 46, 48, 50, 54, 56, 58, 60, 62, 64, 66, 68, 70, 72, 74, 76, 78, 80 or 82, each possibility represents a separate embodiment of the present invention.

According to specific embodiments, the nucleic acid sequence encoding the PD1 polypeptide comprises a nucleic acid sequence selected from the group consisting of SEQ ID NO: 38, 40, 44, 46, 48, 50, 54, 56, 58, 60, 62, 64, 66, 68, 70, 72, 74, 76, 78, 80 and 82.

According to specific embodiments, the nucleic acid sequence encoding the PD1 polypeptide consists of a nucleic acid sequence selected from the group consisting of SEQ ID NO: 38, 40, 44, 46, 48, 50, 54, 56, 58, 60, 62, 64, 66, 68, 70, 72, 74, 76, 78, 80 and 82.

According to specific embodiments, the heterodimer comprises a SIRPα polypeptide.

As used herein the term “SIRPα (Signal Regulatory Protein Alpha, also known as CD172a)” refers to the polypeptide encoded by the SIRPA gene (Gene ID 140885). According to specific embodiments, SIRPα is human SIRPα. According to a specific embodiment, the SIRPα refers to the human SIRPα, such as provided in the following GenBank Number NP_001035111, NP_001035112, NP_001317657 or NP_542970.

According to specific embodiments, SIRPα amino acid sequence comprises SEQ ID NO: 83.

According to specific embodiments, SIRPα amino acid sequence consists of SEQ ID NO: 83.

The known binding pair of SIRPα is CD47. According to a specific embodiment, the CD47 protein refers to the human protein, such as provided in the following GenBank Numbers NP_001768 or NP_942088.

According to specific embodiments, the SIRPα polypeptide binds CD47 with a Kd of 0.1-100 μM, 0.1-10 μM, 1-10 μM, 0.1-5 μM, or 1-2 μM as determined by SPR, each possibility represents a separate embodiment of the present invention.

According to specific embodiments, the SIRPα polypeptide comprises an extracellular domain of said SIRPα or a functional homolog (e.g. fragment) thereof.

According to specific embodiments, SIRPα polypeptide comprises SEQ ID NO: 85 or a functional homolog (e.g. fragment) thereof.

According to specific embodiments, SIRPα polypeptide comprises SEQ ID NO: 85.

According to specific embodiments, SIRPα polypeptide consists of SEQ ID NO: 85.

The term “SIRPα polypeptide” also encompasses functional homologues which exhibit the desired activity (i.e., binding CD47). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 83 or 85 or any other SIRPα amino acid sequence disclosed herein; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

According to specific embodiments, the SIRPα polypeptide may comprise conservative and non-conservative amino acid substitutions. Such substitutions are known in the art and disclosed e.g. in Weiskopf K et al. Science. (2013); 341(6141):88-91, the contents of which are fully incorporated herein by reference.

According to specific embodiments, one or more amino acid mutations are located at an amino acid residue selected from: L4, V6, A21, A27, 131, E47, K53, E54, H56, V63, L66, K68, V92 and F96 corresponding to the SIRPα amino acid sequence set forth in SEQ ID NO: 85.

According to specific embodiments, the SIRPα polypeptide comprises a mutation at an amino acid residue selected from the group consisting of L4, A27, E47 and V92 corresponding to the SIRPα amino acid sequence set forth in SEQ ID NO: 85.

According to specific embodiments, one or more amino acid mutations are selected from the group consisting of: L4V or L4I, V6I or V6L, A21V, A27I or A27L, I31F or I31T, E47V or E47L, K53R, E54Q, H56P or H56R, V63I, L66T or L66G, K68R, V92I and F94L or F94V corresponding to the SIRPα amino acid sequence set forth in SEQ ID NO: 85.

According to specific embodiments, the SIRPα polypeptide comprises a mutation selected from the group consisting of L4I, A27I, E47V and V92I corresponding to the SIRPα amino acid sequence set forth in SEQ ID NO: 85.

As used herein, the phrase “corresponding to the SIRPα amino acid sequence set forth in SEQ ID NO: 85” or “corresponding to SEQ ID NO: 85” intends to include the corresponding amino acid residue relative to any other SIRPα amino acid sequence.

According to specific embodiments, the SIRPα polypeptide comprises SEQ ID NO: 89 or a functional homolog (e.g. fragment) thereof.

According to specific embodiments, the SIRPα polypeptide comprises SEQ ID NO: 89.

According to specific embodiments, the SIRPα polypeptide consists of SEQ ID NO: 89.

Additional description on conservative amino acid and non-conservative amino acid substitutions is further provided hereinabove and below.

The SIRPα polypeptide of some embodiments of the present invention is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 85, 87, 89, 91 or 93; or at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same, each possibility represents a separate embodiment of the present invention.

According to specific embodiments, the SIRPα polypeptide does not comprise the amino acid segment K117-Y343 corresponding to SEQ ID NO: 85.

According to specific embodiments, the SIRPα polypeptide does not comprise any of amino acid residues K117-Y343 corresponding to SEQ ID NO: 85.

According to specific embodiments, the SIRPα polypeptide does not comprise the amino acid segment P118-Y343 corresponding to SEQ ID NO: 85.

According to specific embodiments, the SIRPα polypeptide does not comprise any of amino acid residues P118-Y343 corresponding to SEQ ID NO: 85.

According to specific embodiments, SIRPα polypeptide comprises 100-504, 100-500 amino acids, 150-450 amino acids, 200-400 amino acids, 250-400 amino acids, 300-400 amino acids, 320-420 amino acids, 340-350 amino acids, 300-400 amino acids, 340-450 amino acids, 100-200 amino acids, 100-150 amino acids, 100-125 amino acids, 100-120 amino acids, 100-119 amino acids, 105-119 amino acids, 110-119 amino acids, 115-119 amino acids, 105-118 amino acids, 110-118 amino acids, 115-118 amino acids, 105-117 amino acids, 110-117 amino acids, 115-117 amino acids, each possibility represents a separate embodiment of the present invention.

According to specific embodiments, the SIRPα polypeptide comprises an amino acid sequence selected from the group consisting of SEQ ID NO: 85, 87, 89, 91 and 93 or a functional homolog (e.g. fragment) thereof.

According to specific embodiments, the SIRPα polypeptide comprises an amino acid sequence selected from the group consisting of SEQ ID NO: 85, 87, 89, 91 and 93.

According to specific embodiments, the SIRPα polypeptide consists of an amino acid sequence selected from the group consisting of SEQ ID NO: 85, 87, 89, 91 and 93.

According to specific embodiments, a nucleic acid sequence encoding the SIRPα polypeptide has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 86, 88, 90, 92 or 94, each possibility represents a separate embodiment of the present invention.

According to specific embodiments, the nucleic acid sequence encoding the SIRPα polypeptide comprises a nucleic acid sequence selected from the group consisting of SEQ ID NO: 86, 88, 90, 92 and 94.

According to specific embodiments, the nucleic acid sequence encoding the SIRPα polypeptide consists of a nucleic acid sequence selected from the group consisting of SEQ ID NO: 86, 88, 90, 92 and 94.

According to specific embodiments, the heterodimer comprises a TIGIT polypeptide.

As used herein the term “TIGIT (T Cell Immunoreceptor With Ig And ITIM Domains)” refers to the polypeptide encoded by the TIGIT gene (Gene ID 201633). According to specific embodiments, TIGIT is human TIGIT. According to a specific embodiment, the TIGIT refers to the human TIGIT, such as provided in the following GenBank Number NP_776160 or XP_024309156.

According to specific embodiments, TIGIT amino acid sequence comprises SEQ ID NO: 106.

According to specific embodiments, TIGIT amino acid sequence consists of SEQ ID NO: 106.

A known binding pair of TIGIT is CD155 (PVR). According to a specific embodiment, the CD155 protein refers to the human protein, such as provided in the following GenBank Numbers NP_001129240, NP_001129241, NP_001129242, NP_006496.

According to specific embodiments, the TIGIT polypeptide binds CD155 with a Kd of 0.01-100 μM, 0.1-100 μM, 0.1-10 μM or 0.1-5 μM as determined by SPR, each possibility represents a separate embodiment of the present invention.

According to specific embodiments, the TIGIT polypeptide comprises an extracellular domain of TIGIT or a functional homolog (e.g. fragment) thereof.

According to specific embodiments, the TIGIT polypeptide comprises SEQ ID NO: 107, 113 or 115 or a functional homolog (e.g. fragment) thereof.

According to specific embodiments, the TIGIT polypeptide comprises SEQ ID NO: 107, 113 or 115.

According to specific embodiments, the TIGIT polypeptide consists of SEQ ID NO: 107, 113 or 115.

According to specific embodiments, the TIGIT polypeptide comprises SEQ ID NO: 113.

According to specific embodiments, the TIGIT polypeptide consists of SEQ ID NO: 113.

The term “TIGIT polypeptide” also encompasses functional homologues (which exhibit the desired activity (i.e., binding CD155). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 106, 107, 113 or 115 or any other TIGT amino acid sequence disclosed herein; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

According to specific embodiments, the TIGIT polypeptide may comprise conservative and non-conservative amino acid substitutions.

According to specific embodiments, one or more amino acid mutations are located at an amino acid residue selected from: 142 and C69 corresponding to the TIGIT amino acid sequence set forth in SEQ ID NO: 106.

According to specific embodiments, one or more amino acid mutations are selected from the group consisting of: I42A and C69S corresponding to the TIGIT amino acid sequence set forth in SEQ ID NO: 106.

As used herein, the phrase “corresponding to the TIGIT amino acid sequence set forth in SEQ ID NO: 106” or “corresponding to SEQ ID NO: 106” intends to include the corresponding amino acid residue relative to any other TIGIT amino acid sequence.

According to specific embodiments, the TIGIT polypeptide comprises SEQ ID NO: 109 or 111 or a functional homolog (e.g. fragment) thereof.

According to specific embodiments, the TIGIT polypeptide comprises SEQ ID NO: 109 or 111.

According to specific embodiments, the TIGIT polypeptide consists of SEQ ID NO: 109 or 111.

Additional description on conservative amino acid and non-conservative amino acid substitutions is further provided hereinabove and below.

According to specific embodiments, TIGIT polypeptide comprises 100-244 amino acids, 100-200 amino acids, 100-150 amino acids, 120-140 amino acids, each possibility represents a separate embodiment of the present invention.

According to specific embodiments, a nucleic acid sequence encoding the TIGIT polypeptide has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 108, 110, 112 or 114.

According to specific embodiments, the nucleic acid sequence encoding the TIGIT polypeptide comprises a nucleic acid sequence selected from the group consisting of SEQ ID NO: 108, 110, 112 and 114.

According to specific embodiments, the nucleic acid sequence encoding the TIGIT polypeptide consists of a nucleic acid sequence selected from the group consisting of SEQ ID NO: 108, 110, 112 and 114.

According to specific embodiments, the heterodimer comprises a LILRB2 polypeptide.

As used herein the term “LILRB2 (Leukocyte immunoglobulin-like receptor subfamily B member 2)” refers to the polypeptide encoded by the LILRB2 gene (Gene ID 10288). According to specific embodiments, LILRB2 is human LILRB2. According to a specific embodiment, the LILRB2 refers to the human LILRB2, such as provided in the following GenBank Number NP_001074447, NP_001265332, NP_001265333, NP_001265334, NP_001265335.

A known binding pair of LILRB2 is a major histocompatibility molecule (MHC, e.g. HLA-G). According to specific embodiments, the LILRB2 polypeptide binds MHC (e.g. HLA-G) with a Kd of 0.1 nM-100 μM, 0.1 nM-10 μM, 1 nM-1 μM, 1-100 nM, or 1-10 nM as determined by SPR, each possibility represents a separate embodiment of the present invention.

According to specific embodiments, the LILRB2 polypeptide comprises an extracellular domain of said LILRB2 or a functional homolog (e.g. fragment) thereof.

According to specific embodiments, the LILRB2 polypeptide comprises SEQ ID NO: 95 or a functional homolog (e.g. fragment) thereof.

According to specific embodiments, the LILRB2 polypeptide comprises SEQ ID NO: 95.

According to specific embodiments, the LILRB2 polypeptide consists of SEQ ID NO: 95.

The extracellular domain of LILRB2 comprises 4 Ig-like domains, known as D1-D4.

Hence, according to specific embodiments, the amino acid sequence of LILRB2 polypeptide comprises at least one Ig-like domain.

According to specific embodiments, the LILRB2 polypeptide comprises at least two Ig-like domains, at least three Ig-like domains or four Ig-like domains.

According to specific embodiments, the LILRB2 polypeptide comprises domains D1 and D2 of LILRB2; domains D1, D2 and D3 of LILRB2, domains D1, D2 and D4 or LILRB2, or domains D1, D2, D3 and D4 of LILRB2.

According to specific embodiments, the LILRB2 polypeptide comprises SEQ ID NO: 96 or 98 or a functional homolog (e.g. fragment) thereof.

According to specific embodiments, the LILRB2 polypeptide comprises SEQ ID NO: 96 or 98.

According to specific embodiments, the LILRB2 polypeptide consists of SEQ ID NO: 96 or 98.

The term “LILRB2 polypeptide” also encompasses functional homologues which exhibit the desired activity (i.e., binding MHC, e.g. HLA-G). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 95, 96 or 98 or any other LILRB2 amino acid sequence disclosed herein; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

According to specific embodiments, the LILRB2 polypeptide may comprise conservative and non-conservative amino acid substitutions. Additional description on conservative amino acid and non-conservative amino acid substitutions is further provided hereinabove and below.

According to specific embodiments, LILRB2 polypeptide comprises 100-597 amino acids, 100-500 amino acids, 100-400 amino acids, 150-400 amino acids, 300-400 amino acids, 350-400 amino acids, 150-250 amino acids, each possibility represents a separate embodiment of the present invention.

According to specific embodiments, the LILRB2 polypeptide comprises SEQ ID NO: 96.

According to specific embodiments, the LILRB2 polypeptide consists of SEQ ID NO: 96.

According to specific embodiments, a nucleic acid sequence encoding the LILRB2 polypeptide has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 97 or 99.

According to specific embodiments, the nucleic acid sequence encoding the LILRB2 polypeptide comprises SEQ ID NO: 97.

According to specific embodiments, the nucleic acid sequence encoding the LILRB2 polypeptide consists of SEQ ID NO: 97.

According to specific embodiments, the heterodimer comprises a SIGLEC10 polypeptide.

As used herein the term “SIGLEC-10 (Sialic acid-binding Ig-like lectin 10)” refers to the polypeptide encoded by the SIGLEC10 gene (Gene ID 89790). According to a specific embodiment, the SIGLEC10 refers to the human SIGLEC10, such as provided in the following GenBank Number NP_001164627, NP_001164628, NP_001164629, NP_001164630, NP_001164632.

According to specific embodiments, SIGLEC10 amino acid sequence comprises SEQ ID NO: 100.

According to specific embodiments, SIGLEC amino acid sequence consists of SEQ ID NO: 100.

A known binding pair of SIGLC10 is sialic acid expressed on CD24 and/or CD52. According to specific embodiments, the SIGLEC10 polypeptide binds CD24 or CD52 with a Kd of 1 nM-100 μM, 0.01-100 μM, 0.01-10 μM, 0.1-10 μM, 0.1-5 μM, or 0.1-1 μM as determined by SPR, each possibility represents a separate embodiment of the present invention.

According to specific embodiments, the SIGLEC-10 polypeptide comprises an extracellular domain of SIGLEC10 or a functional homolog (e.g. fragment) thereof.

According to specific embodiments, the SIGLEC10 polypeptide comprises at least one Ig-like domain.

According to specific embodiments, the SIGLEC10 polypeptide comprises at least two Ig-like domain.

According to specific embodiments, the SIGLEC10 polypeptide comprises SEQ ID NO: 105 or a functional homolog (e.g. fragment) thereof.

According to specific embodiments, the SIGLEC10 polypeptide comprises SEQ ID NO: 105.

According to specific embodiments, the SIGLEC10 amino acid sequence consists of SEQ ID NO: 105.

The term “SIGLEC10 polypeptide” also encompasses functional homologues which exhibit the desired activity (i.e., binding sialic acid expressed on CD24 and/or CD52). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 100 or 105; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

According to specific embodiments, the SIGLEC-10 polypeptide may comprise conservative and non-conservative amino acid substitutions.

According to specific embodiments, one mutation is located at an amino acid residue C36 corresponding to the SIGLEC10 amino acid sequence set forth in SEQ ID NO: 100.

According to specific embodiments, one amino acid mutation is C36S corresponding to the SIGLEC10 amino acid sequence set forth in SEQ ID NO: 100.

As used herein, the phrase “corresponding to the SIGLEC10 amino acid sequence set forth in SEQ ID NO: 100” or “corresponding to SEQ ID NO: 100” intends to include the corresponding amino acid residue relative to any other SIGLEC10 amino acid sequence.

According to specific embodiments, the SIGLEC10 polypeptide comprises SEQ ID NO: 103 or a functional homolog (e.g. fragment) thereof.

According to specific embodiments, the SIGLEC10 polypeptide comprises SEQ ID NO: 103.

According to specific embodiments, the SIGLEC-10 polypeptide consists of SEQ ID NO: 103.

Additional description on conservative amino acid and non-conservative amino acid substitutions is further provided hereinabove and below.

According to specific embodiments, SIGLEC10 amino acid sequence comprises 100-639 amino acids, 100-600 amino acids, 100-550 amino acids, 100-300 amino acids, 100-200 amino acids, 100-150 amino acids, each possibility represents a separate embodiment of the present invention.

According to specific embodiments, a nucleic acid sequence encoding the SIGLEC10 polypeptide has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 102 or 104.

According to specific embodiments, the nucleic acid sequence encoding the SIGLEC10 polypeptide comprises SEQ ID NO: 104.

According to specific embodiments, the nucleic acid sequence encoding the SIGLEC10 polypeptide consists of SEQ ID NO: 104.

According to specific embodiments, the heterodimer comprises a SIRPα polypeptide and a PD1 polypeptide, a SIRPα polypeptide and a TIGIT polypeptide, a SIRPα polypeptide and a LILRB2 polypeptide, a SIRPα polypeptide and a SIGLEC10 polypeptide, a PD1 polypeptide and a TIGIT polypeptide, a PD1 polypeptide and a LILRB2 polypeptide, a PD1 polypeptide and a SIGLEC10 polypeptide, a TIGIT polypeptide and a LILRB2 polypeptide, a TIGIT polypeptide and a SIGLEC10 polypeptide or a LILRB2 polypeptide and a SIGLEC10 polypeptide, each possibility represents a separate embodiment of the present invention.

According to specific embodiments, the heterodimer comprises a SIRPα polypeptide and a PD1 polypeptide.

According to a specific embodiment, the heterodimer comprises a SIRPα polypeptide as set forth in SEQ ID NO: 85 and a PD1 polypeptide as set forth in SEQ ID NO: 49.

According to specific embodiments, the heterodimer comprises a SIRPα polypeptide and a LILRB2 polypeptide.

According to a specific embodiment, the heterodimer comprises a SIRPα polypeptide as set forth in SEQ ID NO: 85 and a LILRB2 polypeptide as set forth in SEQ ID NO: 96.

According to a specific embodiment, the heterodimer comprises a SIRPα polypeptide as set forth in SEQ ID NO: 93 and a LILRB2 polypeptide as set forth in SEQ ID NO: 96.

According to specific embodiments, the heterodimer comprises a SIRPα polypeptide and a SIGLEC10 polypeptide.

According to a specific embodiment, the heterodimer comprises a SIRPα polypeptide as set forth in SEQ ID NO: 85 and a SIGLEC10 polypeptide as set forth in SEQ ID NO: 103.

According to specific embodiments, the heterodimer comprises a SIRPα polypeptide and a TIGIT polypeptide.

According to a specific embodiment, the heterodimer comprises a SIRPα polypeptide as set forth in SEQ ID NO: 85 and a TIGIT polypeptide as set forth in SEQ ID NO: 109.

According to specific embodiments, the heterodimer comprises a TIGIT polypeptide and a PD1 polypeptide.

According to a specific embodiment, the heterodimer comprises a TIGIT polypeptide as set forth in SEQ ID NO: 109, 111 or 113 and a PD1 polypeptide as set forth in SEQ ID NO: 49.

According to specific embodiments, the heterodimer comprises a TIGIT polypeptide and a LILRB2 polypeptide.

According to a specific embodiment, the heterodimer comprises a TIGIT polypeptide as set forth in SEQ ID NO: 109 and a LILRB2 polypeptide as set forth in SEQ ID NO: 96.

According to specific embodiments, the heterodimer comprises a TIGIT polypeptide and a SIGLEC10 polypeptide.

According to a specific embodiment, the heterodimer comprises a TIGIT polypeptide as set forth in SEQ ID NO: 109 and a SIGLEC10 polypeptide as set forth in SEQ IS NO: 103.

According to specific embodiments, the heterodimer comprises a PD1 polypeptide and a SIGLEC10 polypeptide.

According to a specific embodiment, the heterodimer comprises a PD1 polypeptide as set forth in SEQ ID NO: 49 and a SIGLEC10 polypeptide as set forth in SEQ ID NO: 103.

According to specific embodiments, the heterodimer comprises a LILRB2 polypeptide and a SIGLEC10 polypeptide.

According to a specific embodiment, the heterodimer comprises a LILRB2 polypeptide as set forth in SEQ ID NO: 96 and a SIGLEC10 polypeptide as set forth in SEQ ID NO: 103.

According to specific embodiments, the heterodimer comprises a PD1 polypeptide and a LILRB2 polypeptide.

According to a specific embodiment, the heterodimer comprises a PD1 polypeptide as set forth in SEQ ID NO: 49 and a LILRB2 polypeptide as set forth in SEQ ID NO: 96.

According to specific embodiments, the heterodimer does not comprise an amino acid sequence of a type II membrane protein capable of binding a natural binding pair thereof.

As used herein, the phrase “an amino acid sequence of a type II membrane protein” refers to a contiguous amino acids sequence of a type II membrane protein capable of at least binding its natural binding pair. According to specific embodiments, such an amino acid sequence comprises an extracellular domain of the type II membrane protein or a functional fragment thereof.

As used herein, the phrase “type II membrane protein” refers to a transmembrane protein having a C-terminus extracellular domain.

Non-limiting examples of such Type II membrane proteins include 4-1BBL, FasL, TRAIL, TNF-alpha, TNF-beta, OX40L, CD40L, CD27L, CD30L, RANKL, TWEAK, APRIL, BAFF, LIGHT, VEGI, GITRL, EDA1/2, Lymphotoxin alpha and Lymphotoxin beta.

According to specific embodiments, the heterodimer does not comprise an amino acid sequence of a type I membrane protein capable of binding a natural binding pair thereof other than the two polypeptides disclosed herein.

As used herein, the phrase “an amino acid sequence of a type I membrane protein” refers to a contiguous amino acids sequence of a type I membrane protein capable of at least binding natural binding pair. According to specific embodiments, such an amino acid sequence comprises an extracellular domain of the type I membrane protein or a functional fragment thereof.

As used herein, the phrase “type I membrane protein” refers to a transmembrane protein having an N-terminus extracellular domain.

Non-limiting examples of such Type I membrane proteins include LAG3, BTN3A1, CD27, CD80, CD86, ENG, NLGN4X, CD84, CD40, IL-8, IL-10, CD164, LY6G6F, CD28, CTLA4, BTLA, LILRB1, TYROBP, ICOS, VEGFA, CSF1, CSF1R, VEGFB, BMP2, BMP3, GDNF, PDGFC, PDGFD, RAET1E, CD155, CD166, MICA, NRG1, HVEM, DR3, TEK, TGFBR (e.g. TGFBR1), LY96, CD96, KIT, CD244 and GFER.

According to specific embodiments, the heterodimer does not comprise a proteinaceous targeting, signaling, immune modulating moiety and/or therapeutic moiety other than the two polypeptides disclosed herein and optionally the dimerizing moiety (e.g. Fc domain of an antibody or a fragment thereof) as further described hereinbelow.

Non-limiting examples of such moieties include a cytokine, a ligand, a receptor, an immune-modulatory polypeptide and a binding domain of an antibody (e.g. ScFv).

According to specific embodiments, the heterodimer consists of the two polypeptides described herein and optionally a dimerizing moiety (e.g. Fc domain of an antibody or a fragment thereof) as further described hereinbelow.

According to other specific embodiments, the heterodimer is attached to or comprises a heterologous therapeutic moiety. The therapeutic moiety may be any molecule, including small molecule chemical compounds and polypeptides.

Non-limiting examples of therapeutic moieties which can be used with specific embodiments of the invention include a cytotoxic moiety, a toxic moiety, a cytokine moiety, an immunomodultory moiety, a polypeptide, an antibody, a drug, a chemical and/or a radioisotope.

According to some embodiments of the invention, the therapeutic moiety is conjugated by translationally fusing the polynucleotide encoding the polypeptide of some embodiments of the invention with the nucleic acid sequence encoding the therapeutic moiety.

Additionally or alternatively, the therapeutic moiety can be chemically conjugated (coupled) to the heterodimer of some embodiments of the invention, using any conjugation method known to one skilled in the art. For example, a peptide can be conjugated to an agent of interest, using a 3-(2-pyridyldithio) propionic acid Nhydroxysuccinimide ester (also called N-succinimidyl 3-(2pyridyldithio) propionate) (“SDPD”) (Sigma, Cat. No. P-3415; see e.g., Cumber et al. 1985, Methods of Enzymology 112: 207-224), a glutaraldehyde conjugation procedure (see e.g., G. T. Hermanson 1996, “Antibody Modification and Conjugation, in Bioconjugate Techniques, Academic Press, San Diego) or a carbodiimide conjugation procedure [see e.g., J. March, Advanced Organic Chemistry: Reaction's, Mechanism, and Structure, pp. 349-50 & 372-74 (3d ed.), 1985; B. Neises et al. 1978, Angew Chem., Int. Ed. Engl. 17:522; A. Hassner et al. 1978, Tetrahedron Lett. 4475; E. P. Boden et al. 1986, J. Org. Chem. 50:2394 and L. J. Mathias 1979, Synthesis 561].

A therapeutic moiety can be attached, for example, to the heterodimer of some embodiments of the invention using standard chemical synthesis techniques widely practiced in the art [see e.g., hypertexttransferprotocol://worldwideweb (dot) chemistry (dot) org/portal/Chemistry)], such as using any suitable chemical linkage, direct or indirect, as via a peptide bond (when the functional moiety is a polypeptide), or via covalent bonding to an intervening linker element, such as a linker peptide or other chemical moiety, such as an organic polymer. Chimeric peptides may be linked via bonding at the carboxy (C) or amino (N) termini of the peptides, or via bonding to internal chemical groups such as straight, branched or cyclic side chains, internal carbon or nitrogen atoms, and the like.

According to specific embodiments, the heterodimer comprises a detectable tag. Hence, according to specific embodiments, any of the polypeptides comprised in the heterodimer may comprise a detectable tag. As used herein, in one embodiment the term “detectable tag” refers to any moiety that can be detected by a skilled practitioner using art known techniques. Detectable tags may be peptide sequences. Optionally the detectable tag may be removable by chemical agents or by enzymatic means, such as proteolysis. Detectable tags of some embodiments of the present invention can be used for purification of the polypeptide or the heterodimer. For example the term “detectable tag” includes chitin binding protein (CBP)-tag, maltose binding protein (MBP)-tag, glutathione-S-transferase (GST)-tag, poly(His)-tag, FLAG tag, Epitope tags, such as, V5-tag, c-myc-tag, and HA-tag, and fluorescence tags such as green fluorescent protein (GFP), red fluorescent protein (RFP), yellow fluorescent protein (YFP), blue fluorescent protein (BFP), and cyan fluorescent protein (CFP); as well as derivatives of these tags, or any tag known in the art. The term “detectable tag” also includes the term “detectable marker”.

According to specific embodiment, the polypeptide comprises a detectable tag attached to its N-terminal (e.g. poly(His)-tag).

According to specific embodiment, the polypeptide comprises a detectable tag attached to its C-terminal (e.g. poly(His)-tag).

According to specific embodiments, the N-terminal of the polypeptide does not comprise a detectable tag (e.g. poly(His)-tag).

According to specific embodiments, the C-terminal of the polypeptide does not comprise a detectable tag (e.g. poly(His)-tag).

According to specific embodiments, the heterodimer comprises a cleavable moiety. Hence, according to specific embodiments, any of the polypeptides comprised in the heterodimer may be fused to a cleavable moiety. Thus, for example, to facilitate recovery, the expressed coding sequence can be engineered to encode the polypeptide of some embodiments of the present invention and fused cleavable moiety. In one embodiment, the polypeptide is designed such that it is readily isolated by affinity chromatography; e.g., by immobilization on a column specific for the cleavable moiety. In one embodiment, a cleavage site is engineered between the polypeptide and the cleavable moiety and the peptide can be released from the chromatographic column by treatment with an appropriate enzyme or agent that specifically cleaves the fusion protein at this site [e.g., see Booth et al., Immunol. Lett. 19:65-70 (1988); and Gardella et al., J. Biol. Chem. 265:15854-15859 (1990)]. According to specific embodiments, the heterodimer comprises a dimerizing moiety attached to the two polypeptides disclosed herein.

As used herein the term “dimerizing moiety” refers to a moiety capable of attaching two different monomers to form a heterodimer. Such dimerizing moieties are known in the art and include chemical and proteinaceous moieties.

According to specific embodiments, the dimerizing moiety is directly attached to the polypeptide.

According to specific embodiments, the dimerizing moiety is non-directly attached to the polypeptide.

According to specific embodiments, the dimerizing moiety is covalently attached to the polypeptide.

According to specific embodiments, the dimerizing moiety is non-covalently attached to the polypeptide.

According to specific embodiments, the dimerizing moiety is heterologous to the polypeptide(s).

According to specific embodiments, the dimerizing moiety is a composition of at least two different molecules.

According to specific embodiments, the dimerizing moiety is capable of activating an immune response upon binding of the heterodimer to a cell expressing a natural binding pair of at least one of the two polypeptides and/or to a cell expressing the natural binding pairs of the two polypeptides.

As used herein, the phrase “activating” refers to stimulation of an immune cell (e.g. T cell, NK cell, B cell, dendritic cell, macrophage) that results in cellular proliferation, maturation, cytokine production and/or induction of regulatory or effector functions. Methods of evaluating immune cell activation or function are well known in the art and include, but are not limited to, proliferation assays such as BRDU and thymidine incorporation, cytotoxicity assays such as chromium release, cytokine secretion assays such as intracellular cytokine staining ELISPOT and ELISA, expression of activation markers such as CD25, CD69 and CD69 using flow cytometry and multimer (e.g. tetramer) assays.

A non-limiting example of such a dimerizing moiety which may be used with specific embodiment is an Fc domain of an antibody, as further described hereinbelow.

According to specific embodiments, the dimerizing moiety is a non-proteinaceous moiety, e.g. a cross linker, an organic polymer, a synthetic polymer, a small molecule and the like.

Numerous such non-proteinaceous moieties are known in the art and can be commercially obtained from e.g. Santa Cruz, Sigma-Aldrich, Proteochem and the like. According to specific embodiments, the non-proteinaceous moiety is a heterobifunctional cross linker. Heterobifunctional cross linkers have two different reactive ends. Typically, in the first step, a monomer is modified with one reactive group of the heterobifunctional reagent; the remaining free reagent is removed. In the second step, the modified monomer is mixed with a second monomer, which is then allowed to react with modifier group at the other end of the reagent. The most widely used couple proteins through amine and sulfhydryl groups (the least stable amine reactive NHS-esters couple first and after removal of uncoupled reagent, the coupling to the sulfhydryl group proceeds). The sulfhydryl reactive groups are generally maleimides, pyridyl disulfides and alpha-halocetyls. Other crosslinkers include carbodiimides, which link between carboxyl groups (—COOH) and primary amines (—NH2). Another approach is to modify the lysine residues of one monomer to thiols and the second monomer is modified by addition of maleimide groups followed by formation of stable thioester bonds between the monomers. If one of the monomers has native thiols, these groups can be reacted directly with maleimide attached to the other monomer. There are also heterobifunational cross-linkers with one phororeactive end, such as Bis[2-(4-azidosalicylamido)ethyl)] disulfide, BASED. Photoreactive groups are used when no specific groups are available to react with—as photoreactive groups react non-specifically upon exposure to UV light. Non-limiting Examples of such heterobifunctional cross linkers include, but are not limited to: Alkyne-PEG4-maleimide, Alkyne-PEG5-N-hydroxysuccinimidyl ester, Maleimide-PEG-succinimidyl ester, Azido-PEG4-phenyloxadiazole methylsulfone, LC-SMCC (succinimidyl-4-(N-maleimidomethyl)cyclohexane-1-carboxy-(6-amidocaproate)), MPBH (4-(4-N-Maleimidophenyl)butyric acid hydrazide hydrochloride+1/2 dioxane), PDPH (3-(2-pyridyldithio)propionyl hydrazide), SIAB (N-succinimidyl (4-iodoacetyl)aminobenzoate), SMPH (succinimidyl-6-((b-maleimidopropionamido)hexanoate), Sulfo-KMUS (N-(κ-maleimidoundecanoyloxy) sulfosuccinimide ester), Sulfo-SIAB (sulfosuccinimidyl (4-iodoacetyl)aminobenzoate), 3-(Maleimido)propionic acid N-hydroxysuccinimide ester, Methoxycarbonylsulfenyl chloride, Propargyl-PEG-acid, Amino-PEG-t-butyl ester, BocNH-PEG5-acid, BMPH (N-(β-maleimidopropionic acid) hydrazide, trifluoroacetic acid salt), ANB-NOS, BMPS, EMCS, GMBS, LC-SPDP, MBS, SBA, SIA, Sulfo-SIA, SMCC, SMPB, SMPH, SPDP, Sulfo-LC-SPDP, Sulfo-MBS, Sulfo-SANPAH, Sulfo-SMCC.

According to other specific embodiments, the dimerizing moiety is a proteinaceous moiety.

According to other specific embodiments, the dimerizing moiety is a proteinaceous dimer moiety.

According to specific embodiments, the polypeptide is attached to an N-terminus of the dimerizing proteinaceous moiety.

According to specific embodiments, the two polypeptides are attached to an N-terminus of the dimerizing proteinaceous moiety.

According to specific embodiments, the polypeptide is attached to a C-terminus of the dimerizing proteinaceous moiety.

According to specific embodiments, the two polypeptides are attached to a C-terminus of the dimerizing proteinaceous moiety.

According to specific embodiments, one of the two polypeptides is attached to an N-terminus of the dimerizing proteinaceous moiety and the second of the two polypeptide is attached to a C-terminus of the dimerizing proteinaceous moiety.

According to specific embodiments, the dimerizing moiety comprises members of affinity pairs polypeptide having two distinct affinity moieties for two different affinity complementary tags. Such affinity pairs are well known in the art and include, but are not limited to hemagglutinin (HA), anti-HA, AviTag™, V5, Myc, T7, FLAG, HSV, VSV-G, His, biotin, avidin, streptavidin, rhizavedin, metal affinity tags, lectins affinity tags. The skilled artisan would know which tag to select.

According to specific embodiments, the dimerizing moiety is an Fc domain of an antibody (e.g., of IgG, IgA, IgD or IgE) or a fragment thereof.

According to specific embodiments, the Fc is of IgG, IgA, IgD or IgE.

According to specific embodiments, the Fc domain of IgG.

According to specific embodiments, the Fc domain is of IgG1 or IgG4.

According to specific embodiments, the Fc domain is of human IgG4. A non-limiting example of human IgG4 Fc domain that can be used with specific embodiments of the invention is provided in SEQ ID NO: 134.

According to specific embodiments, the Fc domain is of human IgG1. Non-limiting examples of human IgG1 Fc domain that can be used with specific embodiments of the invention are provided in SEQ ID NOs: 137 and 156.

According to specific embodiments, the dimerizing moiety is an Fc domain monomer.

According to other specific embodiments, the dimerizing moiety is an Fc domain dimer.

There are a number of mechanisms that can be used to generate a heterodimer using an Fc domain of an antibody, such as, but not limited to, knob-into-hole or charge pairs (see e.g. Gunasekaran et al., J. Biol. Chem. (2010) 285(25):19637, hereby incorporated by reference in its entirety).

Thus, according to specific embodiments, the Fc domain may comprise conservative and non-conservative amino acid substitutions (also referred to herein as mutations).

When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. [Amino acid substitution matrices from protein blocks. Proc. Natl. Acad. Sci. U.S.A. 1992, 89(22): 10915-9].

Additional description on conservative amino acid and non-conservative amino acid substitutions is further provided hereinbelow.

Such substitution in an Fc domain are known in the art.

A representative example, which can be used with specific embodiments of the invention is the “knob-into-hole” (“KTH”) form. Such knob and hole mutations are well known in the art and disclosed e.g. in U.S. Pat. No. 8,216,805, Shane Atwell et Al. J. Mol. Biol. (1997) 270, 26-35; Cater et al. (Protein Engineering vol. 9 no. 7 pp. 617-621, 1996); and A. Margaret Merchant et. al. Nature Biotechnology (1998) 16 Jul., the contents of which are fully incorporated herein by reference. In addition, as described in Merchant et al., Nature Biotech. 16:677 (1998), these “knobs and hole” mutations can be combined with disulfide bonds to skew formation to heterodimerization.

Thus, according to specific embodiments, one of the monomers comprises an Fc domain comprising a knob mutation(s) and the other monomer comprises an Fc domain comprising a hole mutation(s).

It is within the scope of those skilled in the art to select a specific immunoglobulin Fc domain from particular immunoglobulin classes and subclasses and to select a first Fc variant for knob mutation and the other for hole mutation. Non-limiting Examples of substitutions that can be used with specific embodiments include S228P, L235E, T366W, Y349C, T366S, L368A, Y407V and/or E356C [according to EU numbering (Kabat, E. A., T. T. Wu, M. Reid-Miller, H. M. Perry and K. S. Gottesman. 1987. Sequences of proteins of Immunological Interest. US. Dept. of Health and Human Services, Bethesda), corresponding to the human IgG4 as part of a full length antibody], or L235A, Y349C, T366W, T354C, D356C, T366S, L368A and/or Y407V [according to EU numbering (Kabat, E. A., T. T. Wu, M. Reid-Miller, H. M. Perry and K. S. Gottesman. 1987. Sequences of proteins of Immunological Interest. US. Dept. of Health and Human Services, Bethesda) corresponding to the human IgG1 as part of a full length antibody].

Non-limiting examples of IgG4 Fc domains comprising a knob mutation that can be used with specific embodiments of the invention are provided in SEQ ID NOs: 135, 157, 158 and 163.

Non-limiting examples of IgG4 Fc domains comprising a hole mutation that can be used with specific embodiments of the invention are provided in SEQ ID NOs: 136, 159 and 164.

Non-limiting examples of IgG1 Fc domains comprising a knob mutation that can be used with specific embodiments of the invention are provided in SEQ ID NOs: 27, 51, 154 and 160.

Non-limiting examples of IgG1 Fc domains comprising a hole mutation that can be used with specific embodiments of the invention is provided in SEQ ID NOs: 28, 52, 152, 161 and 162.

According to specific embodiments, the Fc domain comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or homology to an amino acid sequence selected from the group consisting of SEQ ID NO: 27, 28, 51, 52, 134, 135, 136, 137, 152, 154, 156, 157, 158, 159, 160, 161, 162, 163 and 164 or a functional fragment thereof which exhibits the desired activity as disclosed herein; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to the polynucleotide sequence encoding same.

According to specific embodiments, the Fc domain comprises an amino acid sequence selected from the group consisting of SEQ ID NO: 135-136, 27-28 or 51-52.

According to specific embodiments, the Fc domain is modified to alter it's binding to an Fc receptor, reduce an immune activating function thereof and/or improve half-life of the fusion.

According to specific embodiments, when the natural binding pair(s) is known to be expressed on healthy cells the Fc domain is modified to reduce it's binding to an Fc receptor and/or an immune activating function thereof.

According to a specific embodiment, the SIRPα-LILRB2 heterodimer is modified to reduce it's binding to an Fc receptor and/or an immune activating function thereof.

According to other specific embodiments, the Fc domain is not-modified to alter it's binding to an Fc receptor, reduce an immune activating function thereof and/or improve half-life of the fusion.

According to specific embodiments, when the natural binding pair(s) is known to be solely expressed or overexpressed on pathologic cells (e.g. cancer cells) the Fc domain is not modified to alter it's binding to an Fc receptor and/or reduce an immune activating function thereof.

According to a specific embodiment, the TIGIT-PD1 heterodimer comprises an Fc domain which is not modified to alter it's binding to an Fc receptor and/or reduce an immune activating function thereof.

According to specific embodiments, the Fc domain is modified to reduce or prevent binding to Fc receptors (e.g. Fc.gamma.RI, Fc.gamma.RII and Fc.gamma.RIII) in vivo. Such modifications have been described by, for example, Clark and colleagues, who have designed and described a series of mutant IgG1, IgG2 and IgG4 Fc domains and their Fc.gamma.R binding properties (Armour et al., 1999; Armour et al., 2002, the content of which are incorporated herein by reference in their entirety). Additional or alternative modifications in the Fc of human IgG1 to reduce it binding to Fc receptors are described by CHAPPEL and colleagues (Proc. Natl. Acad. Sci (1991) 88: 9036-9040, the content of which are incorporated herein by reference in their entirety), who identified amino acids L234 and L235 [according to EU numbering (Kabat et al.) corresponding to a full length antibody] as essential for Fc receptor binding. An additional substitution of P329 to G even weaker the binding, this LALA-PG combination of substitutions is described by e.g., Schlothauer, T., et al. (2016) Protein Eng. Des. Sel. 29, 457-466; and International Patent Application Publication No. WO 2012/130831, the contents of which are incorporated herein by reference in their entirety). Additional or alternative modifications in the Fc of human IgG4 to prevent Fab arm exchange and to reduce it binding to Fc receptor are described by John-Paul Silva et al., (THE JOURNAL OF BIOLOGICAL CHEMISTRY (2015), 290: 9, 5462-5469, the content of which are incorporated herein by reference in their entirety) and Newman et al., (Clinical Immunology (2001) 98:2, the content of which are incorporated herein by reference in their entirety), who identified S228P and L235E [according to EU numbering (Kabat et al.) corresponding to a full length antibody], respectively.

According to specific embodiments, the Fc domain is modified to maximize FcγRIIIa binding. Such modifications have been described by, for example, Shields R L J Biol Chem. (2001) 276:6591, Smith P, Proc Natl Acad Sci USA. (2012) 109:6181, Stavenhagen et al., Cancer Res (2007) 67:8882, Lazar et al., Proc Natl Acad Sci UCA (2006) 103:4005, Richards et al., 2008 Cancer Ther 7:2517 and Mimoto et al., (2013) MAbs 5:229, the content of which are incorporated herein by reference in their entirety. Non-limiting examples of such modifications which can be used with specific embodiments include substitution in one or more amino residues [according to EU numbering (Kabat et al.) corresponding to a full length antibody] selected from S298, E333 and K334 (e.g. S298A, E333A, K334A); G236A, S239A, A330L and 1332E; F243L, R292P, Y300L, V305I and P396L; S239D, 1332E and A330L; 236A, S239D and 1332E; and asymmetric substitution-L234Y/L235Q/G236W/S239M/H268D/D270E/S298A in one heavy chain and D270E/K326D/A330M/K334E in the opposing heavy chain.

According to specific embodiments, the Fc domain is modified to alter effector function, such as to reduce complement binding and/or to reduce or abolish complement dependent cytotoxicity. Such modifications have been described in, for example, U.S. Pat. Nos. 5,624,821 and 5,648,260, 6,194,551, WO 99/51642, Wines et al., 2000, Idusogie et al. (2000) J. Immunol. 164:4178; Tao et al. (1993) J. Exp. Med. 178:661 and Canfield & Morrison (1991) J. Exp. Med. 173: 1483, the content of which are incorporated herein by reference in their entirety. Non-limiting examples of such modifications which can be used with specific embodiments include substitution in one or more amino acids at positions [according to EU numbering (Kabat et al.) corresponding to a full length antibody] selected from 234, 235, 236, 237, 297, 318, 320 and 322; 329, 331 and 322; L234 and/or L235 (e.g. L234A and/or L235A); D270, K322, P329 and P331 (e.g. D270A, K322A, P329A and P331A).

According to specific embodiments, Fc domain is modified to improve the half-life of the fusion protein. Such alterations are described for instance in U.S. Pat. Nos. 5,869,046 and 6,121,022, the content of which are incorporated herein by reference in their entirety. For example, substitution in one or more amino acids at positions [according to EU numbering (Kabat et al.) corresponding to a full length antibody] selected from 252 (e.g., to introduce Thr), 254 (e.g., to introduce Ser) and 256 (e.g., to introduce Phe). Another modification to improve half-life may be by altering the CH1 or CL region to introduce a salvage receptor motif, such as that found in the two loops of a CH2 domain of an Fc region of an IgG.

Maximizing FcRn binding and extending half-life has also been described e.g. in Stapleton N M, Nat Commun. (2011) 2:599, Shields R L. J Biol Chem. (2001) 276:6591, Dall'acqua WF J Immunol. (2002) 169:5171, Zalevsky J, Nat Biotechnol. (2010) 28:157, Ghetie V., Nat. Biotechnol. (1997) 15:637 and Monnet C, MAbs. (2014) 6:422, the content of which are incorporated herein by reference in their entirety. Non-limiting examples of such modifications which can be used with specific embodiments include substitution in one or more amino acids residues [according to EU numbering (Kabat et al.) corresponding to a full length antibody] selected from Arg435His; Asn434Ala; Met252Tyr, Ser254Thr, and Thr256Glu; Met428Leu and Asn434Ser; Thr252Leu, Thr253Ser and Thr254Phe; Glu294delta, Thr307Pro and Asn434Tyr; Thr256Asn, Ala378Val, Ser383Asn and Asn434Tyr.

According to specific embodiments, the dimerizing moiety comprises a leucine zipper or a helix-loop-helix.

According to specific embodiments, each of the moieties comprised in the heterodimer may comprise a linker, separating between the moieties, e.g. between the polypeptide (e.g. SIRPα, PD1, TIGIT, LILRB2, SIGLEC10) and the dimerizing moiety.

According to other specific embodiments, the heterodimer does not comprise a linker between the polypeptide (e.g. SIRPα, PD1, TIGIT, LILRB2, SIGLEC10) and the dimerizing moiety.

Any linker known in the art can be used with specific embodiments of the invention.

According to specific embodiments, the linker may be derived from naturally-occurring multi-domain proteins or is an empirical linker as described, for example, in Chichili et al., (2013), Protein Sci. 22(2): 153-167, Chen et al, (2013), Adv Drug Deliv Rev. 65(10): 1357-1369, the entire contents of which are hereby incorporated by reference. In some embodiments, the linker may be designed using linker designing databases and computer programs such as those described in Chen et al., (2013), Adv Drug Deliv Rev. 65(10): 1357-1369 and Crasto et al., (2000), Protein Eng. 13(5):309-312, the entire contents of which are hereby incorporated by reference.

According to specific embodiments, the linker is a synthetic linker such as PEG.

According to specific embodiments, the linker may be functional. For example, without limitation, the linker may function to improve the folding and/or stability, improve the expression, improve the pharmacokinetics, and/or improve the bioactivity of the heterodimer. In another example, the linker may function to target the heterodimer to a particular cell type or location.

According to specific embodiments, the linker is a polypeptide.

Non-limiting examples of polypeptide linkers include linkers having the sequence LE, GGGGS (SEQ ID NO: 124), (GGGGS)ₙ(n=1-4) (SEQ ID NO: 123), GGGGSGGGG (SEQ ID NO: 122), (GGGGS)x2 (SEQ ID NO: 125), (GGGGS)x2+GGGG (SEQ ID NO: 121), (GGGGS)x3 (SEQ ID NO: 117), (GGGGS)x4 (SEQ ID NO: 118), (Gly)₈ (SEQ ID NO: 119), (Gly)₆ (SEQ ID NO: 120), (EAAAK)ₙ (n=1-3) (SEQ ID NO: 126), A(EAAAK)ₙA (n=2-5) (SEQ ID NO: 127), AEAAAKEAAAKA (SEQ ID NO: 128), A(EAAAK)₄ALEA(EAAAK)4A (SEQ ID NO: 129), PAPAP (SEQ ID NO: 130), K ESGSVSS EQ LAQ FRS LD (SEQ ID NO: 131), EGKSSGSGSESKST (SEQ ID NO: 132), GSAGSAAGSGEF (SEQ ID NO: 133), and (XP)ₙ, with X designating any amino acid, e.g., Ala, Lys, or Glu.

According to specific embodiments, the linker comprises SEQ ID NO: 117 (e.g. but not limited to as a linker between a LILRB2 polypeptide and an Fc domain).

According to specific embodiments, the liner comprises SEQ ID NO: 125 (e.g. but not limited to as a linker between a SIRPα, PD1, TIGIT or SIGLEC10 polypeptide and an Fc domain).

According to specific embodiments, the linker is at a length of one to six amino acids.

According to specific embodiments, the linker is substantially comprised of glycine and/or serine residues (e.g. about 30%, or about 40%, or about 50%, or about 60%, or about 70%, or about 80%, or about 90%, or about 95%, or about 97% or 100% glycines and serines).

According to specific embodiments, the linker is a single amino acid linker.

In some embodiments of the invention, the one amino acid is glycine.

According to specific embodiments, the linker is not an Fc domain or a hinge region of an antibody or a fragment thereof.

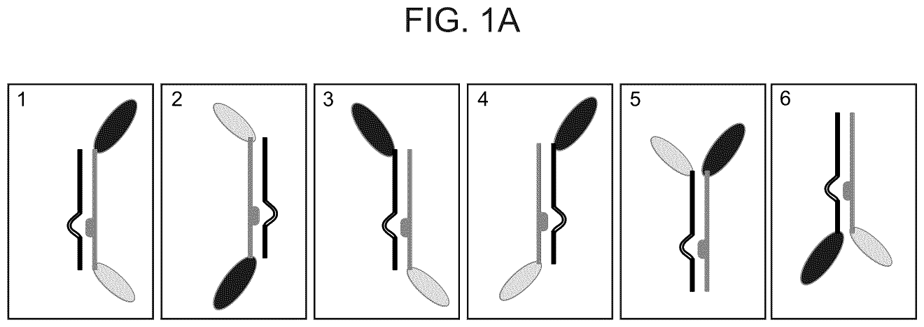

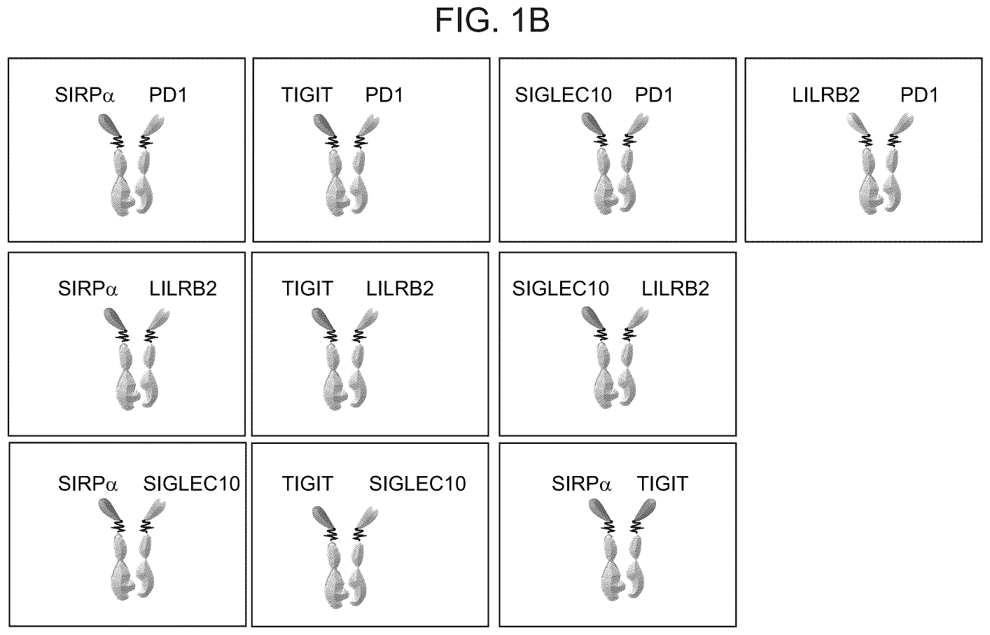

The heterodimer disclosed herein comprises two polypeptides selected from the group consisting of SIRPα, PD1, TIGIT, LILRB2 and SIGLEC10. Non-limiting examples of possible arrangements of such a heterodimer is schematically shown in FIG. 1A.

According to specific embodiments, the heterodimer arrangement is selected from the arrangements shown in panels 1-6 of FIG. 1A, each possibility represents a separate embodiment of the present invention.

According to specific embodiments, the two polypeptides are comprised in a monomer of the heterodimer. Non-limiting examples of such a heterodimer arrangement which may be used with specific embodiments of the invention are shown in panels 1-2 of FIG. 1A.

Hence, according to specific embodiments, one of the monomers of the heterodimer is a fusion polypeptide comprising the two polypeptides.

According to specific embodiments, one of the monomers of the heterodimer is a fusion polypeptide comprising the two polypeptides attached via a proteinaceous dimerizing moiety (e.g. an Fc domain of an antibody or fragment thereof).

As used herein, the term “fusion polypeptide” refers to an amino acid sequence having two or more parts which are not found together in a single amino acid sequence in nature.

According to specific embodiments, one of the monomers of the heterodimer is a fusion polypeptide comprising the two polypeptides attached via an Fc domain of an antibody or fragment thereof comprising a knob mutation(s) and the other monomer comprises an Fc domain of an antibody or fragment thereof comprising a hole mutation(s).

According to specific embodiments, one of the monomers of the heterodimer is a fusion polypeptide comprising the two polypeptides attached via an Fc domain of an antibody or fragment thereof comprising a hole mutation(s) and the other monomer comprises an Fc domain of an antibody or fragment thereof comprising a knob mutation(s).

According to other specific embodiments, each of the two polypeptides is a monomer in the heterodimer. Non-limiting examples of such a heterodimer arrangement which may be used with specific embodiments of the invention are shown in panels 3-6 of FIG. 1A. According to a specific embodiment, the heterodimer arrangement is as shown in panel 5 of FIG. 1A.

According to specific embodiments, the heterodimer comprises a first monomer comprising one of the two polypeptides attached to (e.g., as a translational fusion) a proteinaceous dimerizing moiety (e.g. an Fc domain of an antibody or fragment thereof) and a second monomer comprising the second of the two polypeptides attached to (e.g., as a translational fusion) a proteinaceous dimerizing moiety (e.g. an Fc domain of an antibody or fragment thereof).

According to specific embodiments, the heterodimer comprises a first monomer comprising one of the two polypeptides attached to (e.g., as a translational fusion) an Fc domain of an antibody or fragment thereof comprising a knob mutation(s) and a second monomer comprising the second of the two polypeptides attached to (e.g., as a translational fusion) an Fc domain of an antibody or fragment thereof comprising a hole mutation(s).

According to specific embodiments, the heterodimer composition and arrangement is selected from the heterodimers schematically shown in FIG. 1B, each possibility represents a separate embodiment of the present invention.

Non-limiting examples of heterodimers that can be used with specific embodiments of the present invention are shown in Table 1 hereinbelow.

According to specific embodiments, the heterodimer comprises a monomer comprising a SIRPα polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 1 or 5; and a monomer comprising a PD1 polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 3 or 7.

According to specific embodiments, the heterodimer comprises a monomer comprising SEQ ID NO: 1 and a monomer comprising SEQ ID NO: 3.

According to specific embodiments, the heterodimer comprises a monomer as set forth in SEQ ID NO: 1 and a monomer as set forth in SEQ ID NO: 3.

According to specific embodiments, the heterodimer comprises a monomer comprising SEQ ID NO: 5 and a monomer comprising SEQ ID NO: 7.

According to specific embodiments, the heterodimer comprises a monomer as set forth in SEQ ID NO: 5 and a monomer as set forth in SEQ ID NO: 7.

According to specific embodiments, the heterodimer comprises a monomer comprising a TIGIT polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 9 or 13; and a monomer comprising a LILRB2 polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 11 or 15.

According to specific embodiments, the heterodimer comprises a monomer comprising SEQ ID NO: 9 and a monomer comprising SEQ ID NO: 11.

According to specific embodiments, the heterodimer comprises a monomer as set forth in SEQ ID NO: 9 and a monomer as set forth in SEQ ID NO: 11.

According to specific embodiments, the heterodimer comprises a monomer comprising SEQ ID NO: 13 and a monomer comprising SEQ ID NO: 15.

According to specific embodiments, the heterodimer comprises a monomer as set forth in SEQ ID NO: 13 and a monomer as set forth in SEQ ID NO: 15.

According to specific embodiments, the heterodimer comprises a monomer comprising a SIRPα polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 1, 5, 17, 138, 140, 142 or 144; and a monomer comprising a LLIRB2 polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 11, 15, 19 or 150.

According to specific embodiments, the heterodimer comprises a monomer comprising a SIRPα polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 1, 5 or 17; and a monomer comprising a LLIRB2 polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 11, 15 or 19.

According to specific embodiments, the heterodimer comprises a monomer comprising SEQ ID NO: 1 and a monomer comprising SEQ ID NO: 11.

According to specific embodiments, the heterodimer comprises a monomer as set forth in SEQ ID NO: 1 and a monomer as set forth in SEQ ID NO: 11.

According to specific embodiments, the heterodimer comprises a monomer comprising SEQ ID NO: 5 and a monomer comprising SEQ ID NO: 15.

According to specific embodiments, the heterodimer comprises a monomer as set forth in SEQ ID NO: 5 and a monomer as set forth in SEQ ID NO: 15.

According to specific embodiments, the heterodimer comprises a monomer comprising SEQ ID NO: 17 and a monomer comprising SEQ ID NO: 19.

According to specific embodiments, the heterodimer comprises a monomer as set forth in SEQ ID NO: 17 and a monomer as set forth in SEQ ID NO: 19.

According to specific embodiments, the heterodimer comprises a monomer comprising SEQ ID NO: 138 and a monomer comprising SEQ ID NO: 11.

According to specific embodiments, the heterodimer comprises a monomer as set forth in SEQ ID NO: 138 and a monomer as set forth in SEQ ID NO: 11.

According to specific embodiments, the heterodimer comprises a monomer comprising SEQ ID NO: 140 and a monomer comprising SEQ ID NO: 15.

According to specific embodiments, the heterodimer comprises a monomer as set forth in SEQ ID NO: 140 and a monomer as set forth in SEQ ID NO: 15.

According to specific embodiments, the heterodimer comprises a monomer comprising SEQ ID NO: 142 and a monomer comprising SEQ ID NO: 150.

According to specific embodiments, the heterodimer comprises a monomer as set forth in SEQ ID NO: 142 and a monomer as set forth in SEQ ID NO: 150.

According to specific embodiments, the heterodimer comprises a monomer comprising SEQ ID NO: 144 and a monomer comprising SEQ ID NO: 150.

According to specific embodiments, the heterodimer comprises a monomer as set forth in SEQ ID NO: 144 and a monomer as set forth in SEQ ID NO: 150.

According to specific embodiments, the heterodimer comprises a monomer comprising a LILRB2 polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 21 or 22; and a monomer comprising a PD1 polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 3 or 7.

According to specific embodiments, the heterodimer comprises a monomer comprising SEQ ID NO: 21 and a monomer comprising SEQ ID NO: 3.

According to specific embodiments, the heterodimer comprises a monomer as set forth in SEQ ID NO: 21 and a monomer as set forth in SEQ ID NO: 3.

According to specific embodiments, the heterodimer comprises a monomer comprising SEQ ID NO: 22 and a monomer comprising SEQ ID NO: 7.

According to specific embodiments, the heterodimer comprises a monomer as set forth in SEQ ID NO: 22 and a monomer as set forth in SEQ ID NO: 7.

According to specific embodiments, the heterodimer comprises a monomer comprising a SIGLEC10 polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 24 or 25; and a monomer comprising a PD1 polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 3 or 7.

According to specific embodiments, the heterodimer comprises a monomer comprising SEQ ID NO: 24 and a monomer comprising SEQ ID NO: 3.

According to specific embodiments, the heterodimer comprises a monomer as set forth in SEQ ID NO: 24 and a monomer as set forth in SEQ ID NO: 3.

According to specific embodiments, the heterodimer comprises a monomer comprising SEQ ID NO: 25 and a monomer comprising SEQ ID NO: 7.

According to specific embodiments, the heterodimer comprises a monomer as set forth in SEQ ID NO: 25 and a monomer as set forth in SEQ ID NO: 7.

According to specific embodiments, the heterodimer comprises a monomer comprising a SIRPα polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 1 or 5; and a monomer comprising a SIGLEC10 polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 29 or 30.

According to specific embodiments, the heterodimer comprises a monomer comprising SEQ ID NO: 1 and a monomer comprising SEQ ID NO: 29.

According to specific embodiments, the heterodimer comprises a monomer as set forth in SEQ ID NO: 1 and a monomer as set forth in SEQ ID NO: 29.

According to specific embodiments, the heterodimer comprises a monomer comprising SEQ ID NO: 5 and a monomer comprising SEQ ID NO: 30.

According to specific embodiments, the heterodimer comprises a monomer as set forth in SEQ ID NO: 5 and a monomer as set forth in SEQ ID NO: 30.

According to specific embodiments, the heterodimer comprises a monomer comprising a TIGIT polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 9 or 13; and a monomer comprising a SIGLEC10 polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 29 or 30.

According to specific embodiments, the heterodimer comprises a monomer comprising SEQ ID NO: 9 and a monomer comprising SEQ ID NO: 29.

According to specific embodiments, the heterodimer comprises a monomer as set forth in SEQ ID NO: 9 and a monomer as set forth in SEQ ID NO: 29.

According to specific embodiments, the heterodimer comprises a monomer comprising SEQ ID NO: 13 and a monomer comprising SEQ ID NO: 30.

According to specific embodiments, the heterodimer comprises a monomer as set forth in SEQ ID NO: 13 and a monomer as set forth in SEQ ID NO: 30.

According to specific embodiments, the heterodimer comprises a monomer comprising a SIGLEC10 polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 24 or 25; and a monomer comprising a LILRB2 polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 11 or 15.

According to specific embodiments, the heterodimer comprises a monomer comprising SEQ ID NO: 24 and a monomer comprising SEQ ID NO: 11.

According to specific embodiments, the heterodimer comprises a monomer as set forth in SEQ ID NO: 24 and a monomer as set forth in SEQ ID NO: 11.

According to specific embodiments, the heterodimer comprises a monomer comprising SEQ ID NO: 25 and a monomer comprising SEQ ID NO: 15.

According to specific embodiments, the heterodimer comprises a monomer as set forth in SEQ ID NO: 25 and a monomer as set forth in SEQ ID NO: 15.

According to specific embodiments, the heterodimer comprises a monomer comprising a TIGIT polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 9, 13, 31, 33 or 146; and a monomer comprising a PD1 polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 3, 7 or 148.

According to specific embodiments, the heterodimer comprises a monomer comprising a TIGIT polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 9, 13, 31 or 33; and a monomer comprising a PD1 polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 3 or 7.

According to specific embodiments, the heterodimer comprises a monomer comprising SEQ ID NO: 9 and a monomer comprising SEQ ID NO: 3.

According to specific embodiments, the heterodimer comprises a monomer as set forth in SEQ ID NO: 9 and a monomer as set forth in SEQ ID NO: 3.

According to specific embodiments, the heterodimer comprises a monomer comprising SEQ ID NO: 13 and a monomer comprising SEQ ID NO: 7.

According to specific embodiments, the heterodimer comprises a monomer as set forth in SEQ ID NO: 13 and a monomer as set forth in SEQ ID NO: 7.

According to specific embodiments, the heterodimer comprises a monomer comprising SEQ ID NO: 31 and a monomer comprising SEQ ID NO: 7.

According to specific embodiments, the heterodimer comprises a monomer as set forth in SEQ ID NO: 31 and a monomer as set forth in SEQ ID NO: 7.

According to specific embodiments, the heterodimer comprises a monomer comprising SEQ ID NO: 33 and a monomer comprising SEQ ID NO: 7.

According to specific embodiments, the heterodimer comprises a monomer as set forth in SEQ ID NO: 33 and a monomer as set forth in SEQ ID NO: 7.

According to specific embodiments, the heterodimer comprises a monomer comprising SEQ ID NO: 146 and a monomer comprising SEQ ID NO: 148.

According to specific embodiments, the heterodimer comprises a monomer as set forth in SEQ ID NO: 146 and a monomer as set forth in SEQ ID NO: 148.

According to specific embodiments, the heterodimer comprises a monomer comprising a SIRPα polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 1 or 5; and a monomer comprising a TIGIT polypeptide comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 35 or 36.

According to specific embodiments, the heterodimer comprises a monomer comprising SEQ ID NO: 1 and a monomer comprising SEQ ID NO: 35.

According to specific embodiments, the heterodimer comprises a monomer as set forth in SEQ ID NO: 1 and a monomer as set forth in SEQ ID NO: 35.

According to specific embodiments, the heterodimer comprises a monomer comprising SEQ ID NO: 5 and a monomer comprising SEQ ID NO: 36.

According to specific embodiments, the heterodimer comprises a monomer as set forth in SEQ ID NO: 5 and a monomer as set forth in SEQ ID NO: 36.

According to specific embodiments, the heterodimer disclosed herein is soluble (i.e., not immobilized to a synthetic or a naturally occurring surface).

According to specific embodiments, the heterodimer disclosed herein is immobilized to a synthetic or a naturally occurring surface.

According to an additional or an alternative aspect of the present invention, there is provided a composition comprising the heterodimer disclosed herein, wherein the heterodimer is the predominant form of the two polypeptides in said composition.

Methods of determining dimerization, and specifically heterodimerization, are well known in the art and are further described hereinabove and below.

According to specific embodiments, the predominant form comprises at least 55%, at least 60%, at least 65%, at least 70%, at least 75%, at least 80%, at least 85%, at least 90%, at least 95% or at least 98%, each possibility represents a separate embodiment of the present invention.

According to specific embodiments, the production yield, stability, activity, selectivity and/or safety of the heterodimer or the composition comprising same described herein is higher than the production yield, stability, activity, selectivity and/or safety of a composition comprising a homodimer comprising the same two polypeptides, wherein the homodimer is the predominant form of the two polypeptides in the composition, isolated monomers comprising the same two polypeptides and/or each of the two polypeptides as a single agent.

According to specific embodiments, the production yield, stability, activity, selectivity and/or safety of the heterodimer or the composition comprising same described herein is higher than the production yield, stability, activity, selectivity and/or safety of an antibody e.g. bispecific antibody targeting the natural binding pairs of the two polypeptides described herein.

According to specific embodiments, the increased selectivity and/or safety may be manifested by a selective activity only upon binding of the heterodimer to the natural binding pairs of both polypeptides (e.g. a cell expressing the natural binding pairs of both polypeptides of the heterodimer as compared to a cell expressing only one of the natural binding pairs). In specific embodiments, when the dimerizing moiety is an Fc domain, selectivity and/or safety may be manifested by binding and/or activation of an Fc receptor only upon binding to the natural binding pairs of both polypeptides.

According to specific embodiments, the term “higher” refers to a statistically significant increase.

According to specific embodiments, the term “higher” refers to an increase of at least 1.5 fold, at least 2 fold, at least 2.5 fold, at least 3 fold, at least 5 fold.

According to specific embodiments, the heterodimer or the composition comprising same described herein has a combined improved activity as compared to each of the two polypeptides as a single agent. As used herein the phrase “combined improved activity” refers to at least additive but preferably synergistically improved activity.

According to specific embodiments, the amount of aggregates of the heterodimer or the composition comprising same described herein is at least 20%, at least 30%, at least 40%, at least 50%, at least 60%, at least 70%, at least 80%, at least 90% or at least 95% lower than the amount of aggregates of a composition comprising a homodimer comprising the same two polypeptides, wherein the homodimer is the predominant form of the two polypeptides in the composition, isolated monomers comprising the same two polypeptides and/or each of the two polypeptides as a single agent.

As the heterodimer of some embodiments of present invention comprises two polypeptide elected from SIRPα, PD1, TIGIT, LILRB2 and SIGLEC10, the heterodimer may be used in method of activating immune cells, in-vitro, ex-vivo and/or in-vivo.

Thus, according to an aspect of the present invention, there is provided a method of activating immune cells, the method comprising in-vitro activating immune cells in the presence of the heterodimer, a composition comprising same, a nucleic acid construct or system encoding same or a host cell comprising same.

According to specific embodiments, the immune cells comprise peripheral mononuclear blood cells (PBMCs).

As used herein the term “peripheral mononuclear blood cells (PBMCs)” refers to a blood cell having a single nucleus and includes lymphocytes, monocytes and dendritic cells (DCs).

According to specific embodiments, the PBMCs are selected from the group consisting of dendritic cells (DCs), T cells, B cells, NK cells and NKT cells.

According to specific embodiments, the PBMCs comprise T cells, B cells, NK cells and NKT cells.

Methods of obtaining PBMCs are well known in the art, such as drawing whole blood from a subject and collection in a container containing an anti-coagulant (e.g. heparin or citrate); and apheresis. Following, according to specific embodiments, at least one type of PBMCs is purified from the peripheral blood. There are several methods and reagents known to those skilled in the art for purifying PBMCs from whole blood such as leukapheresis, sedimentation, density gradient centrifugation (e.g. ficoll), centrifugal elutriation, fractionation, chemical lysis of e.g. red blood cells (e.g. by ACK), selection of specific cell types using cell surface markers (using e.g. FACS sorter or magnetic cell separation techniques such as are commercially available e.g. from Invitrogen, Stemcell Technologies, Cellpro, Advanced Magnetics, or Miltenyi Biotec.), and depletion of specific cell types by methods such as eradication (e.g. killing) with specific antibodies or by affinity based purification based on negative selection (using e.g. magnetic cell separation techniques, FACS sorter and/or capture ELISA labeling). Such methods are described for example in THE HANDBOOK OF EXPERIMENTAL IMMUNOLOGY, Volumes 1 to 4, (D. N. Weir, editor) and FLOW CYTOMETRY AND CELL SORTING (A. Radbruch, editor, Springer Verlag, 2000).

According to specific embodiments, the immune cells comprise tumor infiltrating lymphocytes.

As used herein the term “tumor infiltrating lymphocytes (TILs) refers to mononuclear white blood cells that have lest the bloodstream and migrated into a tumor.

According to specific embodiments, the TILs are selected from the group consisting of T cells, B cells, NK cells and monocytes.

Methods of obtaining TILs are well known in the art, such as obtaining tumor samples from a subject by e.g. biopsy or necropsy and preparing a single cell suspension thereof. The single cell suspension can be obtained in any suitable manner, e.g., mechanically (disaggregating the tumor using, e.g., a GentleMACS™ Dissociator, Miltenyi Biotec, Auburn, Calif.) or enzymatically (e.g., collagenase or DNase). Following, the at least one type of TILs can be purified from the cell suspension. There are several methods and reagents known to those skilled in the art for purifying the desired type of TILs, such as selection of specific cell types using cell surface markers (using e.g. FACS sorter or magnetic cell separation techniques such as are commercially available e.g. from Invitrogen, Stemcell Technologies, Cellpro, Advanced Magnetics, or Miltenyi Biotec.), and depletion of specific cell types by methods such as eradication (e.g. killing) with specific antibodies or by affinity based purification based on negative selection (using e.g. magnetic cell separation techniques, FACS sorter and/or capture ELISA labeling). Such methods are described for example in THE HANDBOOK OF EXPERIMENTAL IMMUNOLOGY, Volumes 1 to 4, (D. N. Weir, editor) and FLOW CYTOMETRY AND CELL SORTING (A. Radbruch, editor, Springer Verlag, 2000).

According to specific embodiments, the immune cells comprise phagocytic cells.

As used herein, the term “phagocytic cells” refer to a cell that is capable of phagocytosis and include both professional and non-professional phagocytic cells. Methods of analyzing phagocytosis are well known in the art and include for examples killing assays, flow cytometry and/or microscopic evaluation (live cell imaging, fluorescence microscopy, confocal microscopy, electron microscopy). According to specific embodiments, the phagocytic cells are selected from the group consisting of monocytes, dendritic cells (DCs) and granulocytes.

According to specific embodiments, the phagocytes comprise granulocytes.

According to specific embodiments, the phagocytes comprise monocytes.

According to specific embodiments, the immune cells comprise monocytes.

According to specific embodiments, the term “monocytes” refers to both circulating monocytes and to macrophages (also referred to as mononuclear phagocytes) present in a tissue.

According to specific embodiments, the monocytes comprise macrophages. Typically, cell surface phenotype of macrophages include CD14, CD40, CD11b, CD64, F4/80 (mice)/EMR1 (human), lysozyme M, MAC-1/MAC-3 and CD68.

According to specific embodiments, the monocytes comprise circulating monocytes. Typically, cell surface phenotypes of circulating monocytes include CD14 and CD16 (e.g. CD14++CD16−, CD14+CD16++, CD14++CD16+).

According to specific embodiments, the immune cells comprise DCs

As used herein the term “dendritic cells (DCs)” refers to any member of a diverse population of morphologically similar cell types found in lymphoid or non-lymphoid tissues. DCs are a class of professional antigen presenting cells, and have a high capacity for sensitizing HLA-restricted T cells. DCs include, for example, plasmacytoid dendritic cells, myeloid dendritic cells (including immature and mature dendritic cells), Langerhans cells, interdigitating cells, follicular dendritic cells. Dendritic cells may be recognized by function, or by phenotype, particularly by cell surface phenotype. These cells are characterized by their distinctive morphology having veil-like projections on the cell surface, intermediate to high levels of surface HLA-class II expression and ability to present antigen to T cells, particularly to naive T cells (See Steinman R, et al., Ann. Rev. Immunol. 1991; 9:271-196.). Typically, cell surface phenotype of DCs include CDla+, CD4+, CD86+, or HLA-DR. The term DCs encompasses both immature and mature DCs.

According to specific embodiments, the immune cells comprise granulocytes.

As used herein, the term “granulocytes” refer to polymorphonuclear leukocytes characterized by the presence of granules in their cytoplasm.

According to specific embodiments, the granulocytes comprise neutrophils.

According to specific embodiments, the granulocytes comprise mast-cells.

According to specific embodiments the immune cells comprise T cells.

As used herein, the term “T cells” refers to a differentiated lymphocyte with a CD3+, T cell receptor (TCR)+ having either CD4+ or CD8+ phenotype. The T cell may be either an effector or a regulatory T cell.

As used herein, the term “effector T cells” refers to a T cell that activates or directs other immune cells e.g. by producing cytokines or has a cytotoxic activity e.g., CD4+, Th1/Th2, CD8+ cytotoxic T lymphocyte.

As used herein, the term “regulatory T cell” or “Treg” refers to a T cell that negatively regulates the activation of other T cells, including effector T cells, as well as innate immune system cells. Treg cells are characterized by sustained suppression of effector T cell responses.

According to a specific embodiment, the Treg is a CD4+CD25+Foxp3+ T cell.

According to specific embodiments, the T cells are CD4+ T cells.

According to other specific embodiments, the T cells are CD8+ T cells.

According to specific embodiments, the T cells are memory T cells. Non-limiting examples of memory T cells include effector memory CD4+ T cells with a CD3+/CD4+/CD45RA−/CCR7− phenotype, central memory CD4+ T cells with a CD3+/CD4+/CD45RA−/CCR7+ phenotype, effector memory CD8+ T cells with a CD3+/CD8+CD45RA−/CCR7− phenotype and central memory CD8+ T cells with a CD3+/CD8+CD45RA−/CCR7+ phenotype.

According to specific embodiments, the T cells comprise engineered T cells transduced with a nucleic acid sequence encoding an expression product of interest.

According to specific embodiments, the expression product of interest is a T cell receptor (TCR) or a chimeric antigen receptor (CAR).

As used herein the phrase “transduced with a nucleic acid sequence encoding a TCR” or “transducing with a nucleic acid sequence encoding a TCR” refers to cloning of variable α- and β-chains from T cells with specificity against a desired antigen presented in the context of MHC. Methods of transducing with a TCR are known in the art and are disclosed e.g. in Nicholson et al. Adv Hematol. 2012; 2012:404081; Wang and Riviere Cancer Gene Ther. 2015 March; 22(2):85-94); and Lamers et al, Cancer Gene Therapy (2002) 9, 613-623.

As used herein, the phrase “transduced with a nucleic acid sequence encoding a CAR” or “transducing with a nucleic acid sequence encoding a CAR” refers to cloning of a nucleic acid sequence encoding a chimeric antigen receptor (CAR), wherein the CAR comprises an antigen recognition moiety and a T-cell activation moiety. A chimeric antigen receptor (CAR) is an artificially constructed hybrid protein or polypeptide containing an antigen binding domain of an antibody (e.g., a single chain variable fragment (scFv)) linked to T-cell signaling or T-cell activation domains. Method of transducing with a CAR are known in the art and are disclosed e.g. in Davila et al. Oncoimmunology. 2012 Dec. 1; 1(9):1577-1583; Wang and Riviere Cancer Gene Ther. 2015 March; 22(2):85-94); Maus et al. Blood. 2014 Apr. 24; 123(17):2625-35; Porter D L The New England journal of medicine. 2011, 365(8):725-733; Jackson H J, Nat Rev Clin Oncol. 2016; 13(6):370-383; and Globerson-Levin et al. Mol Ther. 2014; 22(5):1029-1038.

According to specific embodiments, the immune cells comprise B cells.

As used herein the term “B cells” refers to a lymphocyte with a B cell receptor (BCR)+, CD19+ and or B220+ phenotype. B cells are characterized by their ability to bind a specific antigen and elicit a humoral response.

According to specific embodiments, the immune cells comprise NK cells.

As used herein the term “NK cells” refers to differentiated lymphocytes with a CD16+CD56+ and/or CD57+ TCR− phenotype. NK are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

According to specific embodiments, the immune cells comprise NKT cells.

As used herein the term “NKT cells” refers to a specialized population of T cells that express a semi-invariant αβ T-cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD1d. NKT cells can have either protective or deleterious effects due to their abilities to produce cytokines that promote either inflammation or immune tolerance.

According to specific embodiments, the immune cells are obtained from a healthy subject.

According to specific embodiments, the immune cells are obtained from a subject suffering from a pathology (e.g. cancer).

According to specific embodiments, activating is in the presence of cells expressing a natural binding pair of at least one of the two polypeptides or an exogenous binding pair of at least one of the two polypeptides.

According to specific embodiments, activating is in the presence of cells expressing the natural binding pairs of the two polypeptides or exogenous binding pairs of the two polypeptides.

According to specific embodiments, the exogenous binding pair is soluble.

According to other specific embodiments, the exogenous binding pair is immobilized to a solid support.

According to specific embodiments, the cells expressing the binding pair comprise pathologic (diseased) cells, e.g. cancer cells.

According to specific embodiments, the activating is in the presence of a stimulatory agent capable of at least transmitting a primary activating signal [e.g. ligation of the T-Cell Receptor (TCR) with the Major Histocompatibility Complex (MHC)/peptide complex on the Antigen Presenting Cell (APC)] resulting in cellular proliferation, maturation, cytokine production, phagocytosis and/or induction of regulatory or effector functions of the immune cell. According to specific embodiments, the stimulator agent can also transmit a secondary co-stimulatory signal.

Methods of determining the amount of the stimulatory agent and the ratio between the stimulatory agent and the immune cells are well within the capabilities of the skilled in the art and thus are not specified herein.

The stimulatory agent can activate the immune cells in an antigen-dependent or -independent (i.e. polyclonal) manner.

According to specific embodiments, stimulatory agent comprises an antigen non-specific stimulator.

Non-specific stimulators are known to the skilled in the art. Thus, as a non-limiting example, when the immune cells comprise T cells, antigen non-specific stimulator can be an agent capable of binding to a T cell surface structure and induce the polyclonal stimulation of the T cell, such as but not limited to anti-CD3 antibody in combination with a co-stimulatory protein such as anti-CD28 antibody. Other non-limiting examples include anti-CD2, anti-CD137, anti-CD134, Notch-ligands, e.g. Delta-like 1/4, Jagged1/2 either alone or in various combinations with anti-CD3. Other agents that can induce polyclonal stimulation of T cells include, but not limited to mitogens, PHA, PMA-ionomycin, CEB and CytoStim (Miltenyi Biotech). According to specific embodiments, the antigen non-specific stimulator comprises anti-CD3 and anti-CD28 antibodies. According to specific embodiments, the T cell stimulator comprises anti-CD3 and anti-CD28 coated beads, such as the CD3CD28 MACSiBeads obtained from Miltenyi Biotec.

According to specific embodiments, the stimulatory agent comprises an antigen-specific stimulator.

Non-limiting examples of antigen specific T cell stimulators include an antigen-loaded antigen presenting cell [APC, e.g. dendritic cell] and peptide loaded recombinant MHC. Thus, for example, a T cells stimulator can be a dendritic cell preloaded with a desired antigen (e.g. a tumor antigen) or transfected with mRNA coding for the desired antigen.

According to specific embodiments, the antigen is a cancer antigen.

As used herein, the term “cancer antigen” refers to an antigen overexpressed or solely expressed by a cancerous cell as compared to a non-cancerous cell. A cancer antigen may be a known cancer antigen or a new specific antigen that develops in a cancer cell (i.e. neoantigens).

Non-limiting examples for known cancer antigens include MAGE-AI, MAGE-A2, MAGE-A3, MAGE-A4, MAGE-AS, MAGE-A6, MAGE-A7, MAGE-AS, MAGE-A9, MAGE-AIO, MAGE-A11, MAGE-A12, GAGE-I, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-1, LAGE-1, SSX-1, SSX-2(HOM-MEL-40), SSX-3, SSX-4, SSX-5, SCP-1 and XAGE, melanocyte differentiation antigens, p53, ras, CEA, MUCI, PMSA, PSA, tyrosinase, Melan-A, MART-I, gplOO, gp75, alphaactinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAA0205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-I), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, plSOerbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29\BCAA), CA 195, CA 242, CA-50, CAM43, CD68\KP1, C0-029, FGF-5, 0250, Ga733 (EpCAM), HTgp-175, M344, MA-50, MG7-Ag, MOV18, NB\170K, NYCO-I, RCASI, SDCCAG16, TA-90 (Mac-2 binding protein\cyclophilin C-associated protein), TAAL6, TAG72, TLP, TPS, tyrosinase related proteins, TRP-1, or TRP-2.

Other tumor antigens that may be expressed are well-known in the art (see for example WO00/20581; Cancer Vaccines and Immunotherapy (2000) Eds Stern, Beverley and Carroll, Cambridge University Press, Cambridge). The sequences of these tumor antigens are readily available from public databases but are also found in WO 1992/020356 AI, WO 1994/005304 AI, WO 1994/023031 AI, WO 1995/020974 AI, WO 1995/023874 AI & WO 1996/026214 AI.

Alternatively, or additionally, a tumor antigen may be identified using cancer cells obtained from the subject by e.g. biopsy.

Thus, according to specific embodiments, the stimulatory agent comprises a cancer cell.

According to specific embodiments, the activating is in the presence of an anti-cancer agent.

According to specific embodiments, the immune cells are purified following the activation.

Thus, the present invention also contemplates isolated immune cells obtainable according to the methods of the present invention.

According to specific embodiments, the immune cells used and/or obtained according to the present invention can be freshly isolated, stored e.g., cryopreserved (i.e. frozen) at e.g. liquid nitrogen temperature at any stage for long periods of time (e.g., months, years) for future use; and cell lines.

Methods of cryopreservation are commonly known by one of ordinary skill in the art and are disclosed e.g. in International Patent Application Publication Nos. WO2007054160 and WO 2001039594 and US Patent Application Publication No. US20120149108.

According to specific embodiments, the cells obtained according to the present invention can be stored in a cell bank or a depository or storage facility.

Consequently, the present teachings further suggest the use of the isolated immune cells and the methods of the present invention as, but not limited to, a source for adoptive immune cells therapies for diseases that can benefit from activating immune cells e.g. a hyper-proliferative disease; a disease associated with immune suppression and infections.

Thus, according to specific embodiments, a method of the present invention comprises adoptively transferring the immune cells following said activating to a subject in need thereof.

According to specific embodiments, there is provided the immune cells obtainable according to the methods of the present invention for use in adoptive cell therapy.

The cells used according to specific embodiments of the present invention may be autologous or non-autologous; they can be syngeneic or non-syngeneic: allogeneic or xenogeneic to the subject; each possibility represents a separate embodiment of the present invention.

The present teachings also contemplate the use of the compositions of the present invention (e.g. the heterodimer, a composition comprising same, a nucleic acid construct or system encoding same or a host cell expressing same) in methods of treating a disease that can benefit from treatment with the heterodimer.

Thus, according to an aspect of the present invention, there is provided a method of treating a disease that can benefit from treatment with the heterodimer, the method comprising administering to a subject in need thereof the heterodimer, a composition comprising same, a nucleic acid construct or system encoding same or a host cell comprising same, thereby treating the disease in the subject.

According to an additional or an alternative aspect of the present invention, there is provided the heterodimer, a composition comprising same, a nucleic acid construct or system encoding same or a cell comprising same for use in treating a disease that can benefit from treatment with said heterodimer.

The term “treating” or “treatment” refers to inhibiting, preventing or arresting the development of a pathology (disease, disorder or medical condition) and/or causing the reduction, remission, or regression of a pathology or a symptom of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, e.g., human beings at any age and of any gender. According to specific embodiments, the term “subject” refers to a subject who suffers from the pathology (disease, disorder or medical condition). According to specific embodiments, this term encompasses individuals who are at risk to develop the pathology.

According to specific embodiments, cells associated with the disease (e.g. cancer cells) express a natural binding pair of at least one of the two polypeptides.

According to specific embodiments, cells associated with the disease (e.g. cancer cells) express the natural binding pairs of the two polypeptides.

According to specific embodiments, the disease can benefit from activating immune cells.

As used herein the phrase “a disease that can benefit from activating immune cells” refers to diseases in which the subject's immune response activity may be sufficient to at least ameliorate symptoms of the disease or delay onset of symptoms, however for any reason the activity of the subject's immune response in doing so is less than optimal.

Non-limiting examples of diseases that can benefit from activating immune cells include hyper-proliferative diseases, diseases associated with immune suppression, immunosuppression caused by medication (e.g. mTOR inhibitors, calcineurin inhibitor, steroids) and infections.

According to specific embodiments, the disease comprises a hyper-proliferative disease.

According to specific embodiments, the hyper-proliferative disease comprises sclerosis, fibrosis, Idiopathic pulmonary fibrosis, psoriasis, systemic sclerosis/scleroderma, primary biliary cholangitis, primary sclerosing cholangitis, liver fibrosis, prevention of radiation-induced pulmonary fibrosis, myelofibrosis or retroperitoneal fibrosis.

According to other specific embodiments, the hyper-proliferative disease comprises cancer.

As used herein, the term cancer encompasses both malignant and pre-malignant cancers.

With regard to pre-malignant or benign forms of cancer, optionally the compositions and methods thereof may be applied for halting the progression of the pre-malignant cancer to a malignant form.

Cancers which can be treated by the methods of some embodiments of the invention can be any solid or non-solid cancer and/or cancer metastasis.

According to specific embodiments, the cancer comprises malignant cancer.

Cancers which can be treated by the methods of some embodiments of the invention can be any solid or non-solid cancer and/or cancer metastasis. Examples of cancer include but are not limited to, carcinoma, lymphoma, blastoma, sarcoma, and leukemia. More particular examples of such cancers include squamous cell cancer, lung cancer (including small-cell lung cancer, non-small-cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; Burkitt lymphoma, Diffused large B cell lymphoma (DLBCL), high grade lymphoblastic NHL; high-grade small non-cleaved cell NHL; bulky disease NHL; mantle cell lymphoma; AIDS-related lymphoma; and Waldenstrom's Macroglobulinemia); T cell lymphoma, Hodgkin lymphoma, chronic lymphocytic leukemia (CLL); acute lymphoblastic leukemia (ALL); Acute myeloid leukemia (AML), Acute promyelocytic leukemia (APL), Hairy cell leukemia; chronic myeloblastic leukemia (CML); and post-transplant lymphoproliferative disorder (PTLD), as well as abnormal vascular proliferation associated with phakomatoses, edema (such as that associated with brain tumors), and Meigs' syndrome.

According to specific embodiments, the cancer is selected from the group consisting of breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. The cancerous conditions amenable for treatment of the invention include metastatic cancers.

According to specific embodiments, the cancer comprises pre-malignant cancer.

Pre-malignant cancers (or pre-cancers) are well characterized and known in the art (refer, for example, to Berman J J. and Henson D E., 2003. Classifying the precancers: a metadata approach. BMC Med Inform Decis Mak. 3:8). Classes of pre-malignant cancers amenable to treatment via the method of the invention include acquired small or microscopic pre-malignant cancers, acquired large lesions with nuclear atypia, precursor lesions occurring with inherited hyperplastic syndromes that progress to cancer, and acquired diffuse hyperplasias and diffuse metaplasias. Examples of small or microscopic pre-malignant cancers include HGSIL (High grade squamous intraepithelial lesion of uterine cervix), AIN (anal intraepithelial neoplasia), dysplasia of vocal cord, aberrant crypts (of colon), PIN (prostatic intraepithelial neoplasia). Examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Examples of precursor lesions occurring with inherited hyperplastic syndromes that progress to cancer include atypical mole syndrome, C cell adenomatosis and MEA. Examples of acquired diffuse hyperplasias and diffuse metaplasias include AIDS, atypical lymphoid hyperplasia, Paget's disease of bone, post-transplant lymphoproliferative disease and ulcerative colitis.

According to specific embodiments, the cancer is Acute Myeloid Leukemia, Anal Cancer, Basal Cell Carcinoma, B-Cell Non-Hodgkin Lymphoma, Bile Duct Cancer, Bladder Cancer, Breast Cancer, Cervical Cancer, Chronic Lymphocytic Leukemia (CLL), Chronic Myelocytic Leukemia (CML), Colorectal Cancer, Cutaneous T-Cell Lymphoma, Diffuse Large B-Cell Lymphoma, Endometrial Cancer, Esophageal Cancer, Fallopian Tube Cancer, Follicular Lymphoma, Gastric Cancer, Gastroesophageal (GE) Junction Carcinomas, Germ Cell Tumors, Germinomatous (Seminomatous), Germ Cell Tumors, Glioblastoma Multiforme (GBM), Gliosarcoma, Head And Neck Cancer, Hepatocellular Carcinoma, Hodgkin Lymphoma, Hypopharyngeal Cancer, Laryngeal Cancer, Leiomyosarcoma, Mantle Cell Lymphoma, Melanoma, Merkel Cell Carcinoma, Multiple Myeloma, Neuroendocrine Tumors, Non-Hodgkin Lymphoma, Non-Small Cell Lung Cancer, Oral Cavity (Mouth) Cancer, Oropharyngeal Cancer, Osteosarcoma, Ovarian Cancer, Pancreatic Cancer, Peripheral Nerve Sheath Tumor (Neurofibrosarcoma), Peripheral T-Cell Lymphomas (PTCL), Peritoneal Cancer, Prostate Cancer, Renal Cell Carcinoma, Salivary Gland Cancer, Skin Cancer, Small-Cell Lung Cancer, Soft Tissue Sarcoma, Squamous Cell Carcinoma, Synovial Sarcoma, Testicular Cancer, Thymic Carcinoma, Thyroid Cancer, Ureter Cancer, Urethral Cancer, Uterine Cancer, Vaginal Cancer or Vulvar Cancer.

According to specific embodiments, the cancer is Acute myeloid leukemia, Bladder Cancer, Breast Cancer, chronic lymphocytic leukemia, Chronic myelogenous leukemia, Colorectal cancer, Diffuse large B-cell lymphoma, Epithelial Ovarian Cancer, Epithelial Tumor, Fallopian Tube Cancer, Follicular Lymphoma, Glioblastoma multiform, Hepatocellular carcinoma, Head and Neck Cancer, Leukemia, Lymphoma, Mantle Cell Lymphoma, Melanoma, Mesothelioma, Multiple Myeloma, Nasopharyngeal Cancer, Non Hodgkin lymphoma, Non-small-cell lung carcinoma, Ovarian Cancer, Prostate Cancer or Renal cell carcinoma.

According to specific embodiments, the cancer is selected from the group consisting of lymphoma, leukemia and carcinoma (e.g. colon carcinoma, ovarian carcinoma, lung carcinoma, head and neck carcinoma, hepatocellular carcinoma).

According to specific embodiments, the cancer is non-small cell lung cancer (NSCLC).

According to specific embodiments, the cancer is mesothelioma [e.g. malignant pleural mesothelioma (MPM)].

According to specific embodiments, the leukemia is selected from the group consisting of acute nonlymphocytic leukemia, chronic lymphocytic leukemia, acute granulocytic leukemia, chronic granulocytic leukemia, acute promyelocytic leukemia, adult T-cellleukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, ( )ross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

According to specific embodiments, the leukemia is promyelocytic leukemia, acute myeloid leukemia or chronic myelogenous leukemia.

According to specific embodiments, the cancer is lymphoma.

According to specific embodiments, the lymphoma is B cell lymphoma, T cell lymphoma, Hodgkins lymphoma or non-Hodgkins lymphoma.

According to specific embodiments, the non-Hodgkin's Lymphoma is a selected from the group consisting of aggressive NHL, transformed NHL, indolent NHL, relapsed NHL, refractory NHL, low grade non-Hodgkin's Lymphoma, follicular lymphoma, large cell lymphoma, B-cell lymphoma, T-cell lymphoma, Mantle cell lymphoma, Burkitt's lymphoma, NK cell lymphoma, diffuse large B-cell lymphoma, acute lymphoblastic lymphoma, and cutaneous T cell cancer, including mycosos fungoides/Sezry syndrome.

According to specific embodiments, the cancer is multiple myeloma.

According to at least some embodiments, the multiple myeloma is selected from the group consisting of multiple myeloma cancers which produce light chains of kappa-type and/or light chains of lambda-type; aggressive multiple myeloma, including primary plasma cell leukemia (PCL); benign plasma cell disorders such as MGUS (monoclonal gammopathy of undetermined significance), Waldenstrom's macroglobulinemia (WM, also known as lymphoplasmacytic lymphoma) which may proceed to multiple myeloma; smoldering multiple myeloma (SMM), indolent multiple myeloma, premalignant forms of multiple myeloma which may also proceed to multiple myeloma; primary amyloidosis.

According to specific embodiments, the cancer is defined by the presence of tumors that have tumor-infiltrating lymphocytes (TILs) in the tumor micro-environment and/or tumors with a relatively high expression of the natural binding pair in the tumor micro-environment.

According to specific embodiments, the disease comprises a disease associated with immune suppression or immunosuppression caused by medication (e.g. mTOR inhibitors, calcineurin inhibitor, steroids).

According to specific embodiments, the disease comprises HIV, Measles, influenza, LCCM, RSV, Human Rhinoviruses, EBV, CMV or Parvo viruses.

According to specific embodiments, the disease comprises an infection.

As used herein, the term “infection” or “infectious disease” refers to a disease induced by a pathogen. Specific examples of pathogens include, viral pathogens, bacterial pathogens e.g., intracellular mycobacterial pathogens (such as, for example, Mycobacterium tuberculosis), intracellular bacterial pathogens (such as, for example, Listeria monocytogenes), or intracellular protozoan pathogens (such as, for example, Leishmania and Trypanosoma).

Specific types of viral pathogens causing infectious diseases treatable according to the teachings of the present invention include, but are not limited to, retroviruses, circoviruses, parvoviruses, papovaviruses, adenoviruses, herpesviruses, iridoviruses, poxviruses, hepadnaviruses, picornaviruses, caliciviruses, togaviruses, flaviviruses, reoviruses, orthomyxoviruses, paramyxoviruses, rhabdoviruses, bunyaviruses, coronaviruses, arenaviruses, and filoviruses.

Specific examples of viral infections which may be treated according to the teachings of the present invention include, but are not limited to, human immunodeficiency virus (HIV)-induced acquired immunodeficiency syndrome (AIDS), influenza, rhinoviral infection, viral meningitis, Epstein-Barr virus (EBV) infection, hepatitis A, B or C virus infection, measles, papilloma virus infection/warts, cytomegalovirus (CMV) infection, Herpes simplex virus infection, yellow fever, Ebola virus infection, rabies, etc.

According to specific embodiments, the compositions disclosed herein (e.g. heterodimer, composition comprising same, nucleic acid construct or system encoding same and/or host-cell expressing same) can be administered to a subject in combination with other established or experimental therapeutic regimen to treat the disease including, but not limited to analgesics, chemotherapeutic agents, radiotherapeutic agents, cytotoxic therapies (conditioning), hormonal therapy, antibodies and other treatment regimens (e.g., surgery) which are well known in the art.

According to specific embodiments, the therapeutic agent administered in combination with the composition of some embodiments of the invention comprises an antibody.

According to specific embodiments, the compositions disclosed herein (e.g. heterodimer, composition comprising same, nucleic acid construct or system encoding same and/or host-cell expressing same) can be administered to a subject in combination with adoptive cell transplantation such as, but not limited to transplantation of bone marrow cells, hematopoietic stem cells, PBMCs, cord blood stem cells and/or induced pluripotent stem cells.

According to specific embodiments, the therapeutic agent administered in combination with the composition of some embodiments of the invention comprises an anti-cancer agent.

According to specific embodiments, the therapeutic agent administered in combination with the composition of some embodiments of the invention comprises an anti-infection agent (e.g. antibiotics and anti-viral agents).

According to specific embodiments, the therapeutic agent administered in combination with the composition of some embodiments of the invention comprises an immune suppressor agent (e.g. GCSF and other bone marrow stimulators, steroids).

According to specific embodiments the combination therapy has an additive effect.

According to specific embodiments, the combination therapy has a synergistic effect.

According to another aspect of the present invention there is provided an article of manufacture comprising a packaging material packaging a therapeutic agent for treating a disease; and the heterodimer, a composition comprising same, a nucleic acid construct or system encoding same or a host cell comprising same.

According to specific embodiments, the article of manufacture is identified for the treatment of a disease that can benefit from treatment with the heterodimer, e.g. a disease that can benefit from activating immune cells.

According to specific embodiments, the therapeutic agent for treating said disease; and the heterodimer, the composition comprising same, the nucleic acid construct or system encoding same or the host cell expressing same are packaged in separate containers.

According to specific embodiments, the therapeutic agent for treating said disease; and the heterodimer, the composition comprising same, the nucleic acid construct or system encoding same or the host cell expressing same are packaged in a co-formulation.

As used herein, the terms “amino acid sequence”, “protein”, “peptide”, “polypeptide” and “proteinaceous moiety”, which are interchangeably used herein, encompass native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated amide bonds (—N(CH3)-CO—), ester bonds (—C(═O)—O—), ketomethylene bonds (—CO—CH2-), sulfinylmethylene bonds (—S(═O)—CH2-), α-aza bonds (—NH—N(R)—CO—), wherein R is any alkyl (e.g., methyl), amine bonds (—CH2-NH—), sulfide bonds (—CH2-S—), ethylene bonds (—CH2-CH2-), hydroxyethylene bonds (—CH(OH)-CH2-), thioamide bonds (—CS—NH—), olefinic double bonds (—CH═CH—), fluorinated olefinic double bonds (—CF═CH—), retro amide bonds (—NH—CO—), peptide derivatives (—N(R)-CH2-CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides of some embodiments of the invention may also include one or more modified amino acids or one or more non-amino acid monomers (e.g. fatty acids, complex carbohydrates etc.).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 2 and 3 below list naturally occurring amino acids (Table 2), and non-conventional or modified amino acids (e.g., synthetic, Table 3) which can be used with some embodiments of the invention.

TABLE 2

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 3

| Non-conventional | | Non-conventional | |
|---|---|---|---|
| amino acid | Code | amino acid | Code |
| ornithine | Orn | hydroxyproline | Hyp |
| a-aminobutyric acid | Abu | aminonorbornyl- | Norb |
| | | carboxylate | |
| D-alanine | Dala | aminocyclopropane- | Cpro |
| | | carboxylate | |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino-α- | Nmaabu |
| | | methylbutyrate | |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | αmethyl-cyclohexylalanine | Mchexa |
| N-(2-aminoethyl)glycine | Naeg | α-methyl-cyclopentylalanine | Mcpen |
| N-(2,2-diphenylethyl)glycine | Nbhm | N-(N-(2,2-diphenylethyl) | Nnbhm |
| | | carbamylmethyl-glycine | |
| N-(3,3- | Nbhe | N-(N-(3,3-diphenylpropyl) | Nnbhe |
| diphenylpropyl)glycine | | carbamylmethyl-glycine | |
| 1-carboxy-1-(2,2-diphenyl | Nmbc | 1,2,3,4-tetrahydroisoquinoline- | Tic |
| ethylamino)cyclopropane | | 3-carboxylic acid | |
| phosphoserine | pSer | phosphothreonine | pThr |
| phosphotyrosine | pTyr | O-methyl-tyrosine | |
| 2-aminoadipic acid | | hydroxylysine | |

The polypeptides of some embodiments of the invention are preferably utilized in a linear form, although it will be appreciated that in cases where cyclicization does not severely interfere with peptide characteristics, cyclic forms of the peptide can also be utilized.

Since the present heterodimers are preferably utilized in therapeutics which require the heterodimer to be in soluble form, the polypeptides of some embodiments of the invention include one or more non-natural or natural polar amino acids, including but not limited to serine and threonine which are capable of increasing peptide solubility due to their hydroxyl-containing side chain.

The amino acids of the polypeptides of the present invention may be substituted either conservatively or non-conservatively.

The term “conservative substitution” as used herein, refers to the replacement of an amino acid present in the native sequence in the peptide with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions, the substituting amino acid should have the same or a similar functional group in the side chain as the original amino acid.

Conservative substitution tables providing functionally similar amino acids are well known in the art. Guidance concerning which amino acid changes are likely to be phenotypically silent can also be found in Bowie et al., 1990, Science 247: 1306 1310. Such conservatively modified variants are in addition to and do not exclude polymorphic variants, interspecies homologs, and alleles. Typical conservative substitutions include but are not limited to: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). Amino acids can be substituted based upon properties associated with side chains, for example, amino acids with polar side chains may be substituted, for example, Serine (S) and Threonine (T); amino acids based on the electrical charge of a side chains, for example, Arginine (R) and Histidine (H); and amino acids that have hydrophobic side chains, for example, Valine (V) and Leucine (L). As indicated, changes are typically of a minor nature, such as conservative amino acid substitutions that do not significantly affect the folding or activity of the protein.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH [(—CH₂)₅—COOH]—CO— for aspartic acid. Those non-conservative substitutions which fall under the scope of the present invention are those which still constitute a peptide having anti-bacterial properties.

The N and C termini of the peptides of the present invention may be protected by function groups. Suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the compound attached thereto into a cell, for example, by reducing the hydrophilicity and increasing the lipophilicity of the compounds.

According to specific embodiments, one or more of the amino acids may be modified by the addition of a functional group, for example (conceptually views as “chemically modified”). For example, the side amino acid residues appearing in the native sequence may optionally be modified, although as described below alternatively other parts of the protein may optionally be modified, in addition to or in place of the side amino acid residues. The modification may optionally be performed during synthesis of the molecule if a chemical synthetic process is followed, for example by adding a chemically modified amino acid. However, chemical modification of an amino acid when it is already present in the molecule (“in situ” modification) is also possible. Modifications to the peptide or protein can be introduced by gene synthesis, site-directed (e.g., PCR based) or random mutagenesis (e.g., EMS) by exonuclease deletion, by chemical modification, or by fusion of polynucleotide sequences encoding a heterologous domain or binding protein, for example.

As used herein the term “chemical modification”, when referring to a peptide, refers to a peptide where at least one of its amino acid residues is modified either by natural processes, such as processing or other post-translational modifications, or by chemical modification techniques which are well known in the art. Non-limiting exemplary types of modification include carboxymethylation, acetylation, acylation, phosphorylation, glycosylation, amidation, ADP-ribosylation, fatty acylation, addition of farnesyl group, an isofarnesyl group, a carbohydrate group, a fatty acid group, a linker for conjugation, functionalization, GPI anchor formation, covalent attachment of a lipid or lipid derivative, methylation, myristylation, pegylation, prenylation, phosphorylation, ubiquitination, or any similar process and known protecting/blocking groups. Ether bonds can optionally be used to join the serine or threonine hydroxyl to the hydroxyl of a sugar. Amide bonds can optionally be used to join the glutamate or aspartate carboxyl groups to an amino group on a sugar (Garg and Jeanloz, Advances in Carbohydrate Chemistry and Biochemistry, Vol. 43, Academic Press (1985); Kunz, Ang. Chem. Int. Ed. English 26:294-308 (1987)). Acetal and ketal bonds can also optionally be formed between amino acids and carbohydrates. Fatty acid acyl derivatives can optionally be made, for example, by acylation of a free amino group (e.g., lysine) (Toth et al., Peptides: Chemistry, Structure and Biology, Rivier and Marshal, eds., ESCOM Publ., Leiden, 1078-1079 (1990)).

According to specific embodiments, the modifications include the addition of a cycloalkane moiety to the peptide, as described in PCT Application No. WO 2006/050262, hereby incorporated by reference as if fully set forth herein. These moieties are designed for use with biomolecules and may optionally be used to impart various properties to proteins.

Furthermore, optionally any point on the peptide may be modified. For example, pegylation of a glycosylation moiety on a protein may optionally be performed, as described in PCT Application No. WO 2006/050247, hereby incorporated by reference as if fully set forth herein. One or more polyethylene glycol (PEG) groups may optionally be added to O-linked and/or N-linked glycosylation. The PEG group may optionally be branched or linear. Optionally any type of water-soluble polymer may be attached to a glycosylation site on a protein through a glycosyl linker.

By “PEGylated protein” is meant a protein, or a fragment thereof having biological activity, having a polyethylene glycol (PEG) moiety covalently bound to an amino acid residue of the protein.

By “polyethylene glycol” or “PEG” is meant a polyalkylene glycol compound or a derivative thereof, with or without coupling agents or derivatization with coupling or activating moieties (e.g., with thiol, triflate, tresylate, azirdine, oxirane, or preferably with a maleimide moiety). Compounds such as maleimido monomethoxy PEG are exemplary or activated PEG compounds of the invention. Other polyalkylene glycol compounds, such as polypropylene glycol, may be used in the present invention. Other appropriate polyalkylene glycol compounds include, but are not limited to, charged or neutral polymers of the following types: dextran, colominic acids or other carbohydrate-based polymers, polymers of amino acids, and biotin derivatives.

According to specific embodiments, the peptide is modified to have an altered glycosylation pattern (i.e., altered from the original or native glycosylation pattern). As used herein, “altered” means having one or more carbohydrate moieties deleted, and/or having at least one glycosylation site added to the original protein.

Glycosylation of proteins is typically either N-linked or O-linked. N-linked refers to the attachment of the carbohydrate moiety to the side chain of an asparagine residue. The tripeptide sequences, asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to a peptide is conveniently accomplished by altering the amino acid sequence of the peptide such that it contains one or more of the above-described tripeptide sequences (for N-linked glycosylation sites). The alteration may also be made by the addition of, or substitution by, one or more serine or threonine residues in the sequence of the original peptide (for O-linked glycosylation sites). The peptide's amino acid sequence may also be altered by introducing changes at the DNA level.

Another means of increasing the number of carbohydrate moieties on peptides is by chemical or enzymatic coupling of glycosides to the amino acid residues of the peptide. Depending on the coupling mode used, the sugars may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described e.g. in WO 87/05330, and in Aplin and Wriston, CRC Crit. Rev. Biochem., 22: 259-306 (1981).

Removal of any carbohydrate moieties present on a peptide may be accomplished chemically, enzymatically or by introducing changes at the DNA level. Chemical deglycosylation requires exposure of the peptide to trifluoromethanesulfonic acid, or an equivalent compound. This treatment results in the cleavage of most or all sugars except the linking sugar (N-acetylglucosamine or N-acetylgalactosamine), leaving the amino acid sequence intact.

Chemical deglycosylation is described by Hakimuddin et al., Arch. Biochem. Biophys., 259: 52 (1987); and Edge et al., Anal. Biochem., 118: 131 (1981). Enzymatic cleavage of carbohydrate moieties on peptides can be achieved by the use of a variety of endo- and exo-glycosidases as described by Thotakura et al., Meth. Enzymol., 138: 350 (1987).

The polypeptides and heterodimers comprising same of some embodiments of the invention may be synthesized and purified by any techniques that are known to those skilled in the art of peptide synthesis, such as, but not limited to, solid phase and recombinant techniques.

According to specific embodiments, preparing the polypeptide and/or heterodimer involves solid phase peptide synthesis.

For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

Large scale peptide synthesis is described by Andersson Biopolymers 2000; 55(3):227-50.

According to specific embodiments, the polypeptide or heterodimer comprising same is synthesized using in vitro expression systems.

Hence, any of the polypeptides described herein can be encoded from a polynucleotide. These polynucleotides can be used per se or in the recombinant production of the polypeptides disclosed herein.

A “recombinant” polypeptide refers to a polypeptide produced by recombinant DNA techniques; i.e., produced from cells transformed by an exogenous DNA construct encoding the desired polypeptide.

Thus, according to another aspect of the present invention, there is provided a nucleic acid construct or system comprising at least one polynucleotide encoding the heterodimer, and a regulatory element for directing expression of the polynucleotide in a host cell.

Non-limiting examples of polynucleotide sequences which may be used with specific embodiments of the invention are described hereinabove and in Table 1 hereinbelow.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

According to specific embodiments, any of the polynucleotides and nucleic acid sequences disclosed herein may comprise conservative nucleic acid substitutions. Conservatively modified polynucleotides refer to those nucleic acids which encode identical or essentially identical amino acid sequences, or where the nucleic acid does not encode an amino acid sequence, to essentially identical or associated (e.g., naturally contiguous) sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations”, which are one species of conservatively modified polynucleotides. According to specific embodiments, any polynucleotide and nucleic acid sequence described herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a polynucleotide which encodes a polypeptide is implicit in a described sequence with respect to the expression product.

To express an exogenous polypeptide in mammalian cells, a polynucleotide sequence encoding the polypeptide is preferably ligated into a nucleic acid construct suitable for mammalian cell expression. Such a nucleic acid construct includes a promoter sequence for directing transcription of the polynucleotide sequence in the cell in a constitutive or inducible manner.

According to specific embodiments, the regulatory element is a heterologous regulatory element.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, a typical cloning vector may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for secretion of the peptide from a host cell in which it is placed. Preferably the signal sequence for this purpose is a mammalian signal sequence or the signal sequence of the polypeptide variants of some embodiments of the invention.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

Thus, according to specific embodiments, both monomers comprised in the heterodimer are expressed from a single construct.

According to other specific embodiments, each of the monomers comprised in the heterodimer is expressed from a different construct.

It will be appreciated that the individual elements comprised in the expression vector can be arranged in a variety of configurations. For example, enhancer elements, promoters and the like, and even the polynucleotide sequence(s) encoding the monomers or the heterodimer arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo expression of the monomers and heterodimers since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of some embodiments of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

As mentioned, other than containing the necessary elements for the transcription and translation of the inserted coding sequence, the expression construct of some embodiments of the invention can also include sequences engineered to enhance stability, production, purification, yield or toxicity of the expressed monomer or heterodimer. For example, the expression of a fusion protein or a cleavable fusion protein comprising the monomer or heterodimer of some embodiments of the invention and a heterologous protein can be engineered. Such a fusion protein can be designed so that the fusion protein can be readily isolated by affinity chromatography; e.g., by immobilization on a column specific for the heterologous protein. Where a cleavage site is engineered between the monomer or heterodimer of some embodiments of the present invention and the heterologous protein, the monomer or heterodimer can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

The present invention also contemplates cells comprising the composition described herein.

Thus, according to an aspect of the present invention, there is provided a host cell comprising the heterodimer or the nucleic acid construct or system.

As mentioned hereinabove, a variety of prokaryotic or eukaryotic cells can be used as host-expression systems to express the heterodimer of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the polypeptides of some embodiments of the invention.

Examples of bacterial constructs include the pET series of E. coli expression vectors [Studier et al. (1990) Methods in Enzymol. 185:60-89).

Examples of eukaryotic cells which may be used along with the teachings of the invention include but are not limited to, mammalian cells, fungal cells, yeast cells, insect cells, algal cells or plant cells.

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp17.5-E or hsp17.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Other expression systems such as insects and mammalian host cell systems which are well known in the art can also be used by some embodiments of the invention.

According to specific embodiments the cell is a mammalian cell.

According to specific embodiment, the cell is a human cell.

According to a specific embodiment, the cell is a cell line.

According to another specific embodiment, the cell is a primary cell.

The cell may be derived from a suitable tissue including but not limited to blood, muscle, nerve, brain, heart, lung, liver, pancreas, spleen, thymus, esophagus, stomach, intestine, kidney, testis, ovary, hair, skin, bone, breast, uterus, bladder, spinal cord, or various kinds of body fluids. The cells may be derived from any developmental stage including embryo, fetal and adult stages, as well as developmental origin i.e., ectodermal, mesodermal, and endodermal origin.

Non limiting examples of mammalian cells include monkey kidney CV1 line transformed by SV40 (COS, e.g. COS-7, ATCC CRL 1651); human embryonic kidney line (HEK293 or HEK293 cells subcloned for growth in suspension culture, Graham et al., J. Gen Virol., 36:59 1977); baby hamster kidney cells (BHK, ATCC CCL 10); mouse sertoli cells (TM4, Mather, Biol. Reprod., 23:243-251 1980); monkey kidney cells (CV1 ATCC CCL 70); African green monkey kidney cells (VERO-76, ATCC CRL-1587); human cervical carcinoma cells (HeLa, ATCC CCL 2); NIH3T3, Jurkat, canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 1982); MRC 5 cells; FS4 cells; and a human hepatoma line (Hep G2), PER.C6, K562, and Chinese hamster ovary cells (CHO).

According to some embodiments of the invention, the mammalian cell is selected from the group consisting of a Chinese Hamster Ovary (CHO), HEK293, PER.C6, HT1080, NSO, Sp2/0, BHK, Namalwa, COS, HeLa and Vero cell.

According to some embodiments of the invention, the host cell comprises a Chinese Hamster Ovary (CHO), PER.C6 or a 293 (e.g. Expi293F) cell.

According to another aspect of the present invention, there is provided method of producing a heterodimer, the method comprising introducing the nucleic acid construct or system described herein to a host cell or culturing the cells expressing the nucleic acid construct or system described herein.

According to specific embodiments, the producing comprises culturing at 32-37° C., 5-10% CO2 for 5-13 days.

Non-limiting examples of production conditions that can be used with specific embodiments of the invention are disclosed in the Examples section which follows.

Thus, for example an expression vector encoding the heterodimer, is introduced into mammalian cells such as Expi293F, ExpiCHO cells, CHO-K1 or CHO-DG44. The transduced cells are then cultured at 32-37° C. 5-10% CO2 in cell-specific culture medium according to the Expi293F, ExpiCHO, CHO-K1 or CHO-DG44 cells manufacturer instructions (Thermo) and following at least 5 days in culture the proteins are collected from the supernatant and purified.

According to specific embodiments the culture is operated in a batch, split-batch, fed-batch, or perfusion mode.

According to specific embodiments, the culture is operated under fed-batch conditions.

According to specific embodiments, the culturing is effected at 36.5° C.

According to specific embodiments, the culturing it effected at 36. 5° C. with a temperature shift to 32° C. This temperature shift can be effected to slow down cells metabolism prior to reaching a stationary phase.

According to specific embodiments, the method comprising adding the dimerizing moiety to the expressed polypeptides.

According to specific embodiments, the methods comprising isolating the heterodimer.

According to specific embodiments, recovery of the recombinant heterodimer is effected following an appropriate time in culture. According to specific embodiments, recovering the recombinant heterodimer refers to collecting the whole culture medium containing the heterodimer and need not imply additional steps of separation or purification. According to specific embodiments, heterodimers of some embodiments of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, mix mode chromatography, metal affinity chromatography, Lectins affinity chromatography chromatofocusing and differential solubilization.

According to specific embodiments, following production and purification, the therapeutic efficacy of the heterodimer can be assayed either in vivo or in vitro. Such methods are known in the art and include for example cell viability, survival of transgenic mice, and expression of activation markers.

The compositions (e.g. the heterodimer, composition comprising same, nucleic acid construct or system encoding same and/or cells) of some embodiments of the invention can be administered to an organism per se, or in a pharmaceutical composition where it is mixed with suitable carriers or excipients.

Thus, the present invention, in some embodiments, features a pharmaceutical composition comprising a therapeutically effective amount of the composition disclosed herein.

Herein the term “active ingredient” refers to the composition (e.g. heterodimer, composition comprising same, nucleic acid construct or system and/or cells described herein) accountable for the biological effect.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition according to at least some embodiments of the present invention also may include a pharmaceutically acceptable anti-oxidants. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. A pharmaceutical composition according to at least some embodiments of the present invention also may include additives such as detergents and solubilizing agents (e.g., TWEEN 20 (polysorbate-20), TWEEN 80 (polysorbate-80)) and preservatives (e.g., Thimersol, benzyl alcohol) and bulking substances (e.g., lactose, mannitol).

Examples of suitable aqueous and nonaqueous carriers that may be employed in the pharmaceutical compositions according to at least some embodiments of the present invention include water, buffered saline of various buffer content (e.g., Tris-HCl, acetate, phosphate), pH and ionic strength, ethanol, polyols (such as glycerol, propylene glycol, polyethylene glycol, and the like), and suitable mixtures thereof, vegetable oils, such as olive oil, and injectable organic esters, such as ethyl oleate.

Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions according to at least some embodiments of the present invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms according to at least some embodiments of the present invention are dictated by and directly dependent on (a) the unique characteristics of the active compound and the particular therapeutic effect to be achieved, and (b) the limitations inherent in the art of compounding such an active compound for the treatment of sensitivity in individuals.

Techniques for formulation and administration of drugs may be found in “Remington's Pharmaceutical Sciences,” Mack Publishing Co., Easton, PA, latest edition, which is incorporated herein by reference.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

A composition of the present invention can be administered via one or more routes of administration using one or more of a variety of methods known in the art. As will be appreciated by the skilled artisan, the route and/or mode of administration will vary depending upon the desired results. Preferred routes of administration for therapeutic agents according to at least some embodiments of the present invention include intravascular delivery (e.g. injection or infusion), intravenous, intramuscular, intradermal, intraperitoneal, subcutaneous, spinal, oral, enteral, rectal, pulmonary (e.g. inhalation), nasal, topical (including transdermal, buccal and sublingual), intravesical, intravitreal, intraperitoneal, vaginal, brain delivery (e.g. intra-cerebroventricular, intra-cerebral, and convection enhanced diffusion), CNS delivery (e.g. intrathecal, perispinal, and intra-spinal) or parenteral (including subcutaneous, intramuscular, intraperitoneal, intravenous (IV) and intradermal), transdermal (either passively or using iontophoresis or electroporation), transmucosal (e.g., sublingual administration, nasal, vaginal, rectal, or sublingual), administration or administration via an implant, or other parenteral routes of administration, for example by injection or infusion, or other delivery routes and/or forms of administration known in the art. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion or using bioerodible inserts, and can be formulated in dosage forms appropriate for each route of administration. In a specific embodiment, a protein, a therapeutic agent or a pharmaceutical composition according to at least some embodiments of the present invention can be administered intraperitoneally or intravenously.

According to specific embodiments, the compositions disclosed herein are administered in an aqueous solution, by parenteral injection. The formulation may also be in the form of a suspension or emulsion. In general, pharmaceutical compositions for parenteral injection are provided including effective amounts of the compositions described herein, and optionally include pharmaceutically acceptable diluents, preservatives, solubilizers, emulsifiers, adjuvants and/or carriers. Such compositions optionally include one or more for the following: diluents, sterile water, buffered saline of various buffer content (e.g., Tris-HCl, acetate, phosphate), pH and ionic strength; and additives such as detergents and solubilizing agents (e.g., TWEEN 20 (polysorbate-20), TWEEN 80 (polysorbate-80)), anti-oxidants (e.g., water soluble antioxidants such as ascorbic acid, sodium metabisulfite, cysteine hydrochloride, sodium bisulfate, sodium metabisulfite, sodium sulfite; oil-soluble antioxidants, such as ascorbyl palmitate, butylated hydroxyanisole (BHA), butylated hydroxytoluene (BHT), lecithin, propyl gallate, alpha-tocopherol; and metal chelating agents, such as citric acid, ethylenediamine tetraacetic acid (EDTA), sorbitol, tartaric acid, phosphoric acid), and preservatives (e.g., Thimersol, benzyl alcohol) and bulking substances (e.g., lactose, mannitol). Examples of non-aqueous solvents or vehicles are ethanol, propylene glycol, polyethylene glycol, vegetable oils, such as olive oil and corn oil, gelatin, and injectable organic esters such as ethyl oleate. The formulations may be freeze dried (lyophilized) or vacuum dried and redissolved/resuspended immediately before use. The formulation may be sterilized by, for example, filtration through a bacteria retaining filter, by incorporating sterilizing agents into the compositions, by irradiating the compositions, or by heating the compositions.

Various compositions (e.g., polypeptides) disclosed herein can be applied topically. Topical administration does not work well for most peptide formulations, although it can be effective especially if applied to the lungs, nasal, oral (sublingual, buccal), vaginal, or rectal mucosa.

Compositions of the present invention can be delivered to the lungs while inhaling and traverse across the lung epithelial lining to the blood stream when delivered either as an aerosol or spray dried particles having an aerodynamic diameter of less than about 5 microns. A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which may be incorporated into a tablet, gel, capsule, suspension or emulsion. Standard pharmaceutical excipients are available from any formulator. Oral formulations may be in the form of chewing gum, gel strips, tablets or lozenges.

Transdermal formulations may also be prepared. These will typically be ointments, lotions, sprays, or patches, all of which can be prepared using standard technology. Transdermal formulations will require the inclusion of penetration enhancers. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

According to specific embodiments, the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans. Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

In certain embodiments, the composition (e.g. heterodimer, composition comprising same, the nucleic acid construct or system or cells) is administered locally, for example by injection directly into a site to be treated. Typically, the injection causes an increased localized concentration of the composition which is greater than that which can be achieved by systemic administration. The heterodimer compositions can be combined with a matrix as described above to assist in creating an increased localized concentration of the polypeptide compositions by reducing the passive diffusion of the polypeptides out of the site to be treated.

Pharmaceutical compositions of the present invention may be administered with medical devices known in the art. For example, in an optional embodiment, a pharmaceutical composition according to at least some embodiments of the present invention can be administered with a needle hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of the active agents disclosed herein, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl Polymer ScL, 35:755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

In certain embodiments, to ensure that the therapeutic compounds according to at least some embodiments of the present invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J Physiol. 1233:134); p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Reagents—ExcelBand™ 3-color high range protein marker or ExcelBand™ 3-color extra range protein marker (SMOBIO, Cat #PM2600 or PM2800 respectively), Sample buffer (GenScript Cat #M00676), Polyacrylamide gel 8% or 4-20% GenScript Cat #M00662 or M00656 respectively), ECL Plus Western Blotting substrate (Pierce, Cat #32132), TMB-ELISA Substrate Solution (Sigma, Cat #T0440), TMB stop solution (Southern Biotech, Cat #0412-01), Streptavidin-HRP (Pierce Cat #TS21126). FuGENE® HD Transfection Reagent (Promega, Cat #TM328). Vybrant DiD cell labeling solution (Thermo Fisher, Cat #V22887), Lymphoprep, ™ Density gradient medium (StemCells Technologies, Cat #07801).

Antibodies—LEAF™ purified Mouse anti human PD-L1 (CD274) B7H1 clone 29E.2A3, BioLegend, Cat #329711, Anti-human PD1 (GenScript, Cat #A01829-40), APC-labeled anti PD1 (Biolegend, Cat #329908), APC Mouse IgG2b, κ IC (Biolegend, Cat #400322), Biotinylated rabbit anti-human SIRPα (LsBio Cat #LS-C370337), Rabbit anti-human SIRPα antibody (anti-drug antibody DSP107) Batch #104, Anti-human LILRB2 (R&D systems Cat #MAB2078), APC anti-human CD155 antibody (Biolegend, Cat #337618), APC Mouse IgG1 k, (Biolegend, Cat #400120), APC anti-human CD47 antibody (Biolegend, Cat #323124), APC anti-human CD274 (Biolegend, Cat #329708), APC anti-human CD172a/b SIRPα clone SE5A5 Antibody, Mouse IgG1, K, (Biolegend, Cat #323809), PE mouse anti-human IgG1-Fc, (SouthernBiotech, Cat #9054-09), PE Mouse anti-human IgG4 (SouthernBiotech, Cat #9190-09), AF647 anti-human IgG4-Fc, (SouthernBiotech Cat #9200-31), APC anti-human CD85d (ILT4) antibody clone 41D1 (Biolegend, Cat #338708), Goat anti rabbit IgG (H+L)-HRP conjugate (R&D systems, Cat #170-6515), Goat anti-mouse IgG HRP conjugate (Bio-rad Cat #170-6516), APC anti human HLA-G (from patent US 2020/0102390 A1), APC human IgG4 (Biolegend, Cat #403706), CD47 blocker Ab (from patent WO 2011/143624 A2), HLA-G blocker Ab (from patent US 2020/0102390 A1), Mouse anti-human IgG1 HRP conjugate (Southern Biotech, Cat #9054-05), Mouse anti-human IgG4 HRP conjugate (Southern Biotech, Cat #9200-05), PE Mouse anti-Human IgG1 FC (Southern Biotech, Cat #9054-09), PE mouse anti-human IgG4-Fc (SouthernBiotech, Cat #9190-09), Anti-human PVR (NOVUS, Cat #NB6001241), APC Mouse anti-human IgG1 hinge (Southern Biotech, Cat #9054-09), APC Anti human TIGIT, mIgG2a clone A15153G (Biolegend, Cat #372706), APC mIgG2a MOPC-173 (Biolegend, Cat #400220), PE Anti human PD-L1, 29E2A3, mIgG2b (Biolegend, Cat #329706), APC Anti human CD16, mIgG1 clone ICRF44 (Biolegend, Cat #301310).

Recombinant proteins—Human recombinant HLA-G protein (His tag), (Abcam Cat #ab225660), Human PDL-1 FC Tag (Acro Cat #PD1-H5258), Human PDL-1-Fc (GenScript U3420DK140-1), Human CD47 protein HIS Tag (Acro, Cat #CD7-H5227), Human CD155 (PVR) protein Fc Tag (Acro Cat #CD5-H5251 and CD5-H5254).

Cell lines—Expi 293F (Gibco, Cat #A14257), DLD1-WT cell line (ATCC, CCL-221), DLD1-PDL1 cell lines (Hendriks et al 2016), HT1080 WT (ATCC CCL-121), CHO-K1, CHO-K1-CD47, CHO-K1-CD47 (Cell Bank Australia, CBA-0146), HT1080-HLA-G (cells were generated by virus infection with HLA-G expression plasmid), JEG-3 cells (ATCC, HTB-36), K562 (ATCC, CCL-243), K562 PVR, K562 PD-L1, K562 PVR/PD-L1 (produced by transfection of K562 cells with PDL1 and/or PVR expressing plasmids and selection for stable expressing cells), AB12 (ECACC, 10092306), Renca (ATCC, CRL-2947), Jurkat NFAT-CD16 (Promega, jktl-nfat-cd16), SK-OV-3 (ATCC, HTB-77).

Media and Tissue culture reagents—Expi 269 medium (Gibco, Cat #A-14351-01), RPMI 1640 (Biological Industries, Cat #01-100-1A), FCS (Gibco, Cat #12657-029), BSA, Sigma, A7030, EDTA, Sigma, E7889, Cell Dissociation Buffer, Gibco, 13151-014, EMEM (Biological Industries, Cat #01-040-1A), DMEM (Biological Industries, Cat #01-055-1A), TrypLE Express (Gibco, Cat #12604-13), Glutamax (Gibco, Cat #35050-038), Penicillin-Streptomycin (Gibco, Cat #151140-122), Sodium pyruvate (Biological Industries, Cat #03-042-1B), IMDM (Biological Industries, Cat #01-058-1A).

Equipment—FACS Device, Stratadigm, Cytometry S1000EXI, ELISA Reader, Thermoscience Multiskan FC, ELISA Software, SkanIt Software 4.1 for Microplate Readers RE, ver. 4.1.0.43.

Structural analysis of heterodimers-proteins—Homology modeling was performed for each part based on a homologue X-ray structure. For PD1—PDB IDs: 3RRQ, 5GGR, 5GGS, 5JXE and 4ZQK were used as templates. For hIgG4—PDB IDs: 4C54, 4C55, 5W5M and 5W5N were used as templates. For SIRPα—PDB ID's 2UV3, 2WNG, 4CMM, 6BIT, 2JJS and 2JJT were used as templates. For LILRB2—PDB IDs: 2GW5, 4LLA, 2DYP and 6BCP were used as templates. For SIGLEC10—PDB IDs: 2N7A and 2N7B of the SIGLEC8 homologue were used as templates. For TIGIT—PDB IDs: 3QOH, 3RQ3, 3UCR, 3UDW and 5V52 were used as templates.

Linker segments were modeled using loop modeling in CHARMM primarily in order to avoid structural violations and to enable a plausible estimation for a possible ‘spacer’ length.

Manufacturing of heterodimers—For comparative functional analysis and production evaluation, several heterodimers (referred to herein as “DSPs”) were designed and/or produced using the “knob” into “hole” method (described e.g. in U.S. Pat. No. 8,216,805), see Table 1 hereinbelow. Production was effected in Expi293F cells transfected by pcDNA3.4 expression vectors cloned with coding sequence for the desired Fc fusion proteins (see Table 1 hereinbelow).

The sequences were cloned into the vector using restriction enzymes such as EcoRI and HindIII or XbaI and EcoRV, with addition of Kozak sequence and STOP codon plus an artificial signal peptide (MESPAQLLFLLLLWLPDGVHA, SEQ ID NO: 116). The proteins were collected from the supernatant of cell culture and in some cases, proteins were purified by one-step purification using protein A (PA) Poros MabCapture A resin or Anion Exchange High Trap Q FF resin.

TABLE 1
Description of the designed heterodimers

| | First monomer | | Second monomer | |
|---|---|---|---|---|
| Heterodimer | Description | Sequences aa/na | Description | Sequences aa/na |
| DSP120 | SIRPα-IgG1 | 1 (85 + 51)/2 | PD1-IgG1 | 3 (49 + 52)/4 |
| | knob Fc | | hole Fc | |
| DSP120V1 | SIRPα-IgG4 | 5 (85 + 135)/6 | PD1-IgG4 | 7 (49 + 136)/8 |
| | knob Fc | | hole Fc | |
| DSP205 | TIGIT- IgG1 | 9 (109 + 51)/10 | LILRB2- | 11 (96 + 52)/12 |
| | knob Fc | | IgG1 hole Fc | |
| DSP205V1 | TIGIT- IgG4 | 13 (109 + 135)/14 | LILRB2- | 15 (96 + 136)/16 |
| | knob Fc | | IgG4 hole Fc | |
| DSP216 | SIRPα-IgG1 | 1 (85 + 51)/2 | LILRB2- | 11 (96 + 52)/12 |
| | knob Fc | | IgG1 hole Fc | |
| DSP216V1 | SIRPα-IgG4 | 5 (85 + 135)/6 | LILRB2- | 15 (96 + 136)/16 |
| | knob Fc | | IgG4 hole Fc | |
| DSP216V2 | SIRPα-IgG1 | 17 (85 + 27)/18 | LILRB2- | 19 (96 + 28)/20 |
| | knob Fc | | IgG1 hole Fc | |
| DSP220 | LILRB2-IgG1 | 21 (96 + 51) | PD1-IgG1 | 3 (49 + 52)/4 |
| | knob Fc | | hole Fc | |
| DSP220V1 | LILRB2-IgG4 | 22 (96 + 135)/23 | PD1-IgG4 | 7 (49 + 136)/8 |
| | knob Fc | | hole Fc | |
| DSP402 | SIGLEC10- | 24 (103 + 51) | PD1-IgG1 | 3 (49 + 52)/4 |
| | IgG1 knob Fc | | hole Fc | |
| DSP402V1 | SIGLEC10- | 25 (103 + 135)/26 | PD1-IgG4 | 7 (49 + 136)/8 |
| | IgG4 knob Fc | | hole Fc | |
| DSP403 | SIRPα-IgG1 | 1 (85 + 51)/2 | SIGLEC10- | 29 (103 + 52) |
| | knob Fc | | IgG1 hole Fc | |
| DSP403V1 | SIRPα-IgG4 | 5 (85 + 135)/6 | SIGLEC10- | 30 (103 + 136) |
| | knob Fc | | IgG4 hole Fc | |
| DSP404 | TIGIT-IgG1 | 9 (109 + 51)/10 | SIGLEC10- | 29 (103 + 52) |
| | knob Fc | | IgG1 hole Fc | |
| DSP404V1 | TIGIT- IgG4 | 13 (109 + 135)/14 | SIGLEC10- | 30 (103 + 136) |
| | knob Fc | | IgG4 hole Fc | |
| DSP412 | SIGLEC10- | 24 (103 + 51) | LILRB2- | 11 (96 + 52)/12 |
| | IgG1 knob Fc | | IgG1 hole Fc | |
| DSP412V1 | SIGLEC10- | 25 (103 + 135)/26 | LILRB2- | 15 (96 + 136)/16 |
| | IgG4 knob Fc | | IgG4 hole Fc | |
| DSP502 | TIGIT-IgG1 | 9 (109 + 51)/10 | PD1-IgG1 | 3 (49 + 52)/4 |
| | knob Fc | | hole Fc | |
| DSP502V1 | TIGIT-IgG4 | 13 (109 + 135)/14 | PD1-IgG4 | 7 (49 + 136)/8 |
| | knob Fc | | hole Fc | |
| DSP502V2 | TIGIT-IgG4 | 31 (111 + 135)/32 | PD1-IgG4 | 7(49 + 136)/8 |
| | knob Fc | | hole Fc | |
| DSP502V3 | TIGIT-IgG4 | 33 (113 + 135)/34 | PD1-IgG4 | 7(49 + 136)/8 |
| | knob Fc | | hole Fc | |
| DSP503 | SIRPα-IgG1 | 1 (85 + 51)/2 | TIGIT-IgG1 | 35 (109 + 52) |
| | knob Fc | | hole Fc | |
| DSP503V1 | SIRPα-IgG4 | 5 (85 + 135)/6 | TIGIT-IgG4 | 36 (109 + 136) |
| | knob Fc | | hole Fc | |
| DSP216V3 | SIRPα-short- | 138 (93 + 51)/139 | LILRB2- | 11 (96 + 52)/12 |
| | IgG1 knob Fc | | IgG1 hole Fc | |
| DSP216V4 | SIRPα-short- | 140 (93 + 135)8/141 | LILRB2- | 15 (96 + 136)/16 |
| | IgG4 knob Fc | | IgG4 hole Fc | |
| DSP216V5 | SIRPα-IgG1 | 142 (85 + 154)/143 | LILRB2- | 150 (96 + 152)/151 |
| | knob Fc- | | IgG1 hole | |
| | LALA | | Fc-LALA | |
| DSP216V6 | SIRPα-short- | 144(93 + 154)/145 | LILRB2- | 150 (96 + 152)/151 |
| | IgG1 knob Fc- | | IgG1 hole | |
| | LALA | | Fc-LALA | |
| DSP502V4 | TIGIT-IgG1 | 146 (109 + 154)/147 | PD1-IgG1 | 148 (49 + 152)/149 |
| | knob Fc- | | hole Fc- | |
| | LALA | | LALA | |

SDS-PAGE analysis—Thirty-five μl of cell culture supernatant or 3 μg purified protein from each heterodimer sample were mixed with loading buffer with or without β-mercaptoethanol (reduced and non-reduced conditions, respectively), heated for 5 minutes at 95° C. and separated on 8% or 4-20% gradient polyacrylamide gel electrophoresis SDS-PAGE. Proteins migration on the gel was visualized by e-Stain machinery (GenScript), according to manufacturer instructions.

Western blot analysis—Samples containing the produced heterodimers (50-500 ng per lane) were treated at reducing or non-reducing conditions (in loading buffer with or without 03-mercaptoethanol, respectively), heated for 5 minutes at 95° C. and separated on a 8% or 4-20% gradient SDS-PAGE. Following, proteins were transferred onto a PVDF membrane and incubated with primary antibodies for one hour or overnight, followed by 1 hour incubation with an HRP-conjugated secondary antibody. Signals were detected following ECL development.

Binding of heterodimers to their counterpart ligands/receptors by Sandwich ELISA—Flat bottom 96-wells plates were pre-coated with receptor/ligand of one arm of the analyzed-heterodimer by incubating overnight at 4° C. with a recombinant counterpart protein such as CD47 protein, PDL1 protein, HLA-G protein, or PVR protein, followed by blocking and washing. Serially diluted cell culture supernatant or purified protein samples containing the produced heterodimers were added to the corresponding pre-coated wells. Following an additional washing step, biotinylated, an unlabeled Ab or HRP-labeled Ab against the second arm of the heterodimer or the IgG backbone was added and allowed to bind to the captured protein. Thereafter, in case where the detected Ab was not HRP-labeled, HRP conjugated secondary Ab or streptavidin-HRP was added. Detection was effected with a TMB substrate according to standard ELISA protocol using a plate reader (Thermo Scientific, Multiscan FC) at 450 nm, with reference at 620 nm. O.D. values were used to create a binding curve graph with a GraphPad Prism software.

Binding of heterodimers to their counterpart-ligands/receptors expressed on cell's surface—Cells expressing one of the analyzed-heterodimer's counterparts were incubated with serial dilutions of the produced heterodimer for 30 minutes at 4° C., followed by immuno-staining with fluorescently labeled antibody specific to the second component of the heterodimer (i.e., the one that does not bind the counterpart expressed by the cells) or to the IgG backbone and analysis by flow cytometry. Optionally, in cases that the cells express both counterparts, cells underwent pre-incubation with a blocker antibody against one of them prior to incubation with the heterodimer. O.D. values were used to create a binding curve graph with a GraphPad Prism software.

The effect of the heterodimers comprising SIRPα and LILRB2 domain on Macrophages and PMN cells—To test the phagocytosis of cancer cells by granulocytes HT1080 expressing human CD47 or HT1080-HLA-G cells expressing human CD47 and human HLA-G were labelled with DiD and pre-incubated with 0, 1, 2 or 5 μg/mL DSP216 for 15 minutes at RT. Following, the cells were co-cultured in a effector to target (E:T) ratio 1:1 with granulocytes overnight at 37° C. and analyzed by Flow cytometer. MFI values were used to create a binding curve graph with a GraphPad Prism software.

Cytotoxicity assay—Killing of Target cells by NK cells (effector cells) was tested in a co-culture assay. Percentage of dead target cells was analyzed by flow cytometry analysis (FACS) following overnight incubation. Pre-labeled target cells [K562 WT, K562 overexpressing PVR (herein K562 PVR), K562 overexpressing PDL1 (herein K562 PDL1) or K562 overexpressing PVR/PDL1 (herein K562 PVR/PDL1) cells] were placed in 96-wells plates and incubated with primary NK cells at various effector-target (E:T) ratios in the presence of different concentrations of a TIGIT-PD1 heterodimer. Cells were analyzed by flow cytometry following ON incubation at 37° C. K562 WT target cells were used as a reference to the inhibition of the killing effect in the presence of PVR/PD-L1 ligands expressed on the target cells. Reference for the TIGIT-PD1 heterodimer treatment was the non-treated target cells.

Secretion of Granzyme B—Granzyme B is a serine protease most commonly found in the granules of NK cells and cytotoxic T cells. It is secreted by these cells along with the pore forming protein perforin to mediate apoptosis in target cells. Prelabeled target cells (K562 WT, K562 PVR, K562 PDL1 and K562 PVR/PDL1 overexpressing cells) were placed in 96-well plates and incubated with primary NK cells at various effector-target (E:T) ratios in the presence of different concentrations of a TIGIT-PD1 heterodimer. Following ON incubation at 37° C., the supernatant was collected and analyzed for Granzyme B levels using ELISA.

FcγRIIIa binding using Luciferase activity assay by a reporter gene system—FcγRIIIa activation by IgG1-Fc was detected using a reporter system of Jurkat NFAT CD16 overexpressing cells (Promega). In this system, binding to the FcγRIIIa induces a cascade of signal transaction which includes: increase in intracellular calcium levels and activation of the calcium-sensitive phosphatase, calcineurin, which rapidly de-phosphorylates NFAT proteins. De-phosphorylated NFAT translocates into the nucleus and induces luciferase expression and secretion. The level of Luciferase secretion was measured as a luminescence signal, produced by interaction of luciferase and added substrate (QUANTI-Luc). Binding of the IgG1-Fc arm of the TIGIT-PD1 heterodimer to FcγRIIIa (CD16) on Jurkat CD16 overexpressing cells was tested in a co-culture assay of K562 PVR/PDL-1 cells (Target cells) with Jurkat NFAT-CD16 overexpressing cells (Effector cells). To this end, K562 PVR/PD-L1 were cultured in 96-well plates and incubated in the presence of different concentrations of a TIGIT-PD1 heterodimer. Following 1 hour incubation at 37° C., Jurkat NFAT CD16 overexpressing cells were added to the target cells, in an E:T ratio of 2:1. Luciferase activity was analyzed by Luminometer following 6 hours incubation at 37° C. K562 WT target cells were used as a reference to the luciferase activity in the presence of PVR/PD-L1 ligands expressed on the target cells. Control for the TIGIT-PD1 heterodimer treatment was the non-treated target cells.

Simultaneous binding of TIGIT-PD1 heterodimer to its counterparts, as determined by ELISA—Flat bottom 96-wells plates were pre-coated with hrPDL1-Fc protein, followed by blocking and washing. Serially diluted purified TIGIT-PD1 heterodimer samples were added to the corresponding pre-coated wells. Following incubation and an additional washing step, a detection protein—human CD155 (PVR) conjugate to mouse IgG2a Fc, was added and allowed to bind to the captured protein. Followed an additional wash, peroxidase-conjugated AffiniPure Goat Anti-Mouse IgG was added and after incubation and wash detection was effected with a TMB substrate according to a standard ELISA protocol using a plate reader (Thermo Scientific, Multiscan FC) at 450 nm, with reference at 620 nm.

Simultaneous Binding of a TIGIT-PD1 heterodimer to NK cells and tumor cells—Simultaneous binding of TIGIT and PD1 to NK and tumor cells was tested in a co-culture cell system of NK primary cells and K562 PVR/PD-L1 cells in the presence of a TIGIT-PD1 heterodimer. The simultaneous binding of the two moieties to the different cell types leads to doublets formation that was detected by flow cytometry as a double positive stained cell-population. To allow detection of doublet cells formation a co-culture of primary NK cells pre-labeled with CPD dye was mixed with K562 PVR/PD-L1 cells pre-labeled with a CFSE dye, in a ratio of 2:1. Doublet cells formation was tested in the presence of different concentrations of a TIGIT-PD1 heterodimer, following 2 hours incubation at 4° C. In addition, the specificity of doublet cells formation was tested following 1 hour incubation with blocking Abs: Fc blocker, PVR Ab or PD-L1 Ab, prior to the addition of the heterodimer.

In vivo Syngeneic Model: the effect of a TIGIT-PD1 heterodimer in an AB12 Mouse Mesothelioma Cancer Model—7 weeks old female BALB/c mice were inoculated intraperitoneally (i.p.) with AB12 mouse malignant pleural mesothelioma (MPM) cells followed by treatment with a TIGIT-PD1 heterodimer or vehicle control. The effect of the heterodimer was evaluated by its influence on mice survival.

### Treatment Protocol:

10⁵ AB12 cells were inoculated i.p. on day 0 of the study.
   Two groups were assigned in the experiment:
Group 1—Following inoculation, starting from day 6, mice were treated i.p. with 200 μl of vehicle/mouse. Vehicle was administered every 3 days.
Group 2—Following inoculation, starting from day 6, mice were treated i.p. with 150 μg TIGIT-PD1 heterodimer in a volume of 200 μl. A total of 4 doses were given in intervals of 3 days.
   The animals were evaluated daily for morbidity and mortality.

In vivo NSG Model: the effect of a TIGIT-PD1 heterodimer in an A549 Human Lung Adenocarcinoma Cancer Model—11-13 weeks old NSG mice were inoculated subcutaneous (s.c.) with human lung adenocarcinoma cells followed by treatment with a TTGTT-PD1 heterodimer or vehicle control. The effect of the heterodimer was evaluated by assessing tumor volume and calculating tumor growth inhibition (TGI).

### Treatment Protocol:

5.0×10⁶ A549 cells were inoculated s.c. on day 0 of the study. On day 9, mice were irradiated and human PBMCs (5×10⁶) were injected i.v. followed by a second injection of human PBMCs (5×10⁶) on day 16.
   Two groups were assigned in the experiment:
Group 1—mice were injected i.p. with 200 μl/mouse PBS every other day (EOD) starting at day 9.
Group 2—mice were treated i.p. with 150 μg TIGIT-PD1 heterodimer in a volume of 200 μl/mice EOD starting from day 9. A total of 4 doses of were given.
   Tumor volumes were measured from the first treatment day (day 9) and EOD during the follow up period. All animals were sacrificed on day 18.

### Example 1

### Selection, Design, Production and Characterization of Heterodimers Comprising Two Fc Fusion Proteins

Several heterodimers comprising two proteins selected from SIRPα, PD1, TIGIT, LILRB2 and SIGLEC10, wherein each of the proteins is fused to an Fc domain of IgG1 or IgG4 using the “knob” into “hole” method (described e.g. in U.S. Pat. No. 8,216,805), were designed (See Table 1 hereinabove)

Structural analysis of heterodimers-proteins was effected in order to optimize the following parameters:
Folding—proper folding to allow binding to targets, minimize potential di-sulfide scrambling.
Integrity—no exposed proteolytic sites.
High expression in mammalian expression system; and
Low immunogenicity.

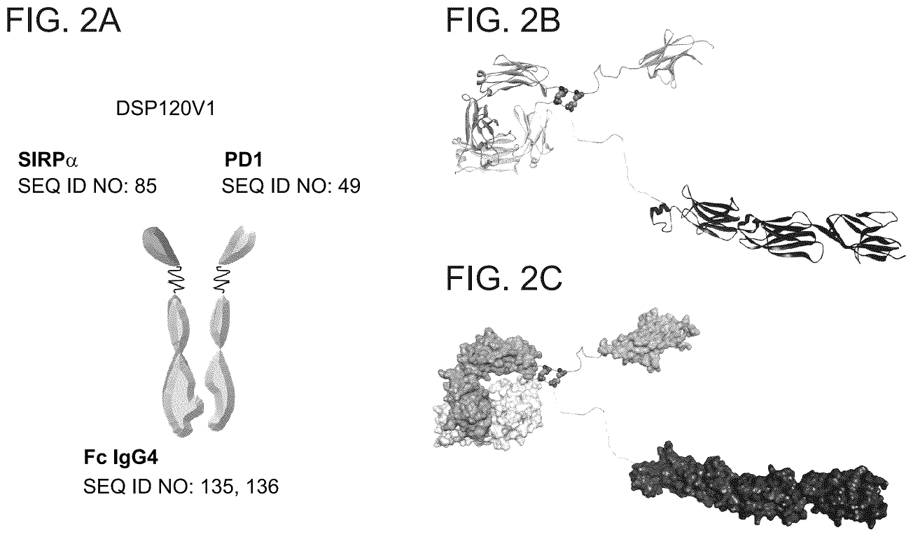

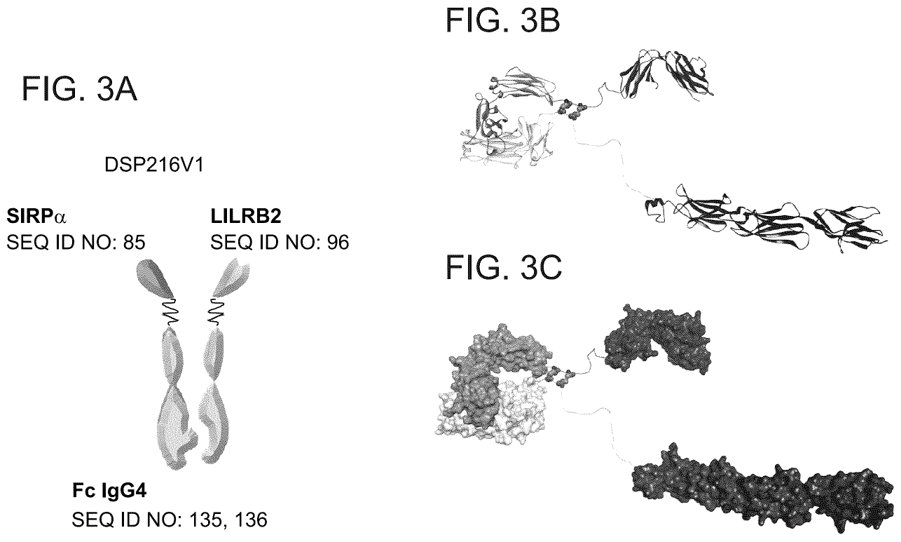

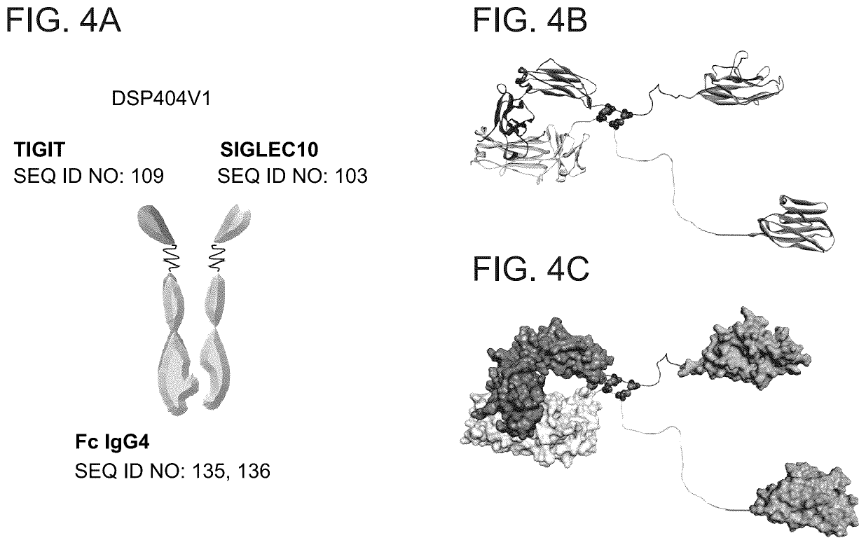

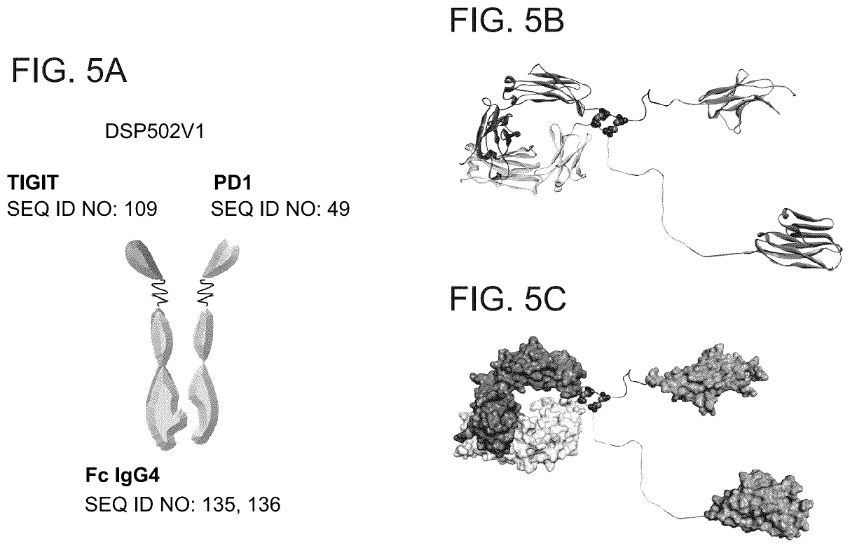

FIGS. 2A-5C present schematic drawings of the heterodimers referred to herein as “DSP120V1”, “DSP216V1”, “DSP404V1”, “DSP502V1” (see description and sequences in Table 1 hereinabove) and the 3D models generated for the domains and segments identified. This analysis predicted possible binding to the ligands and no interference between the different domains.

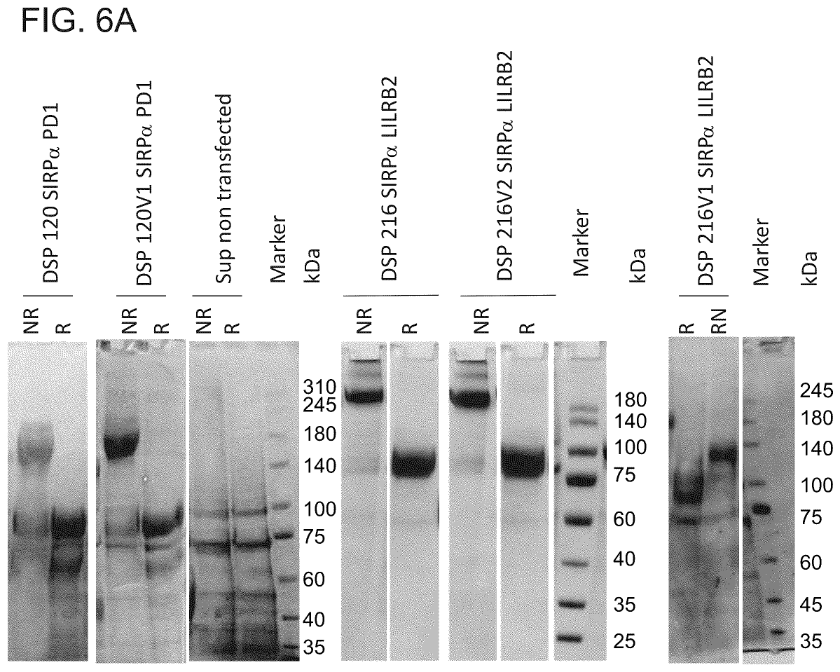

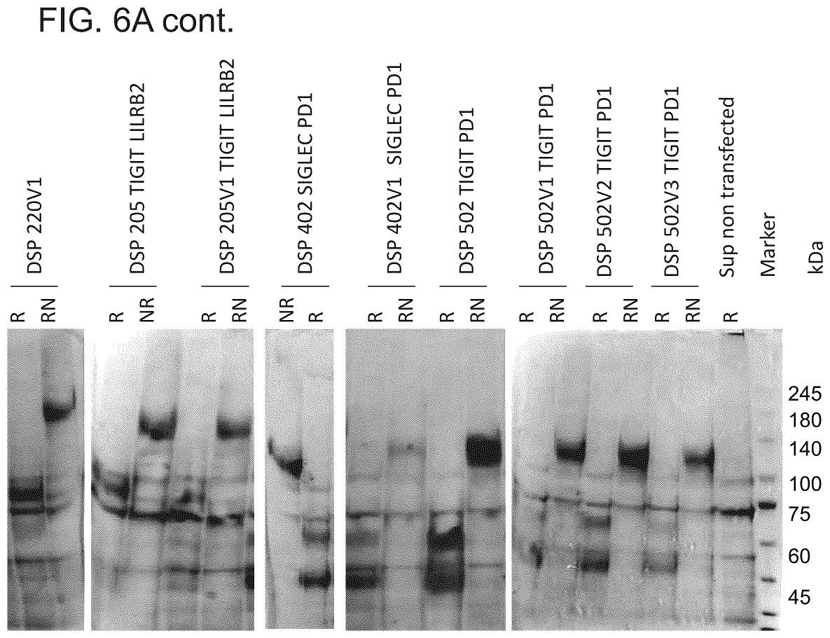

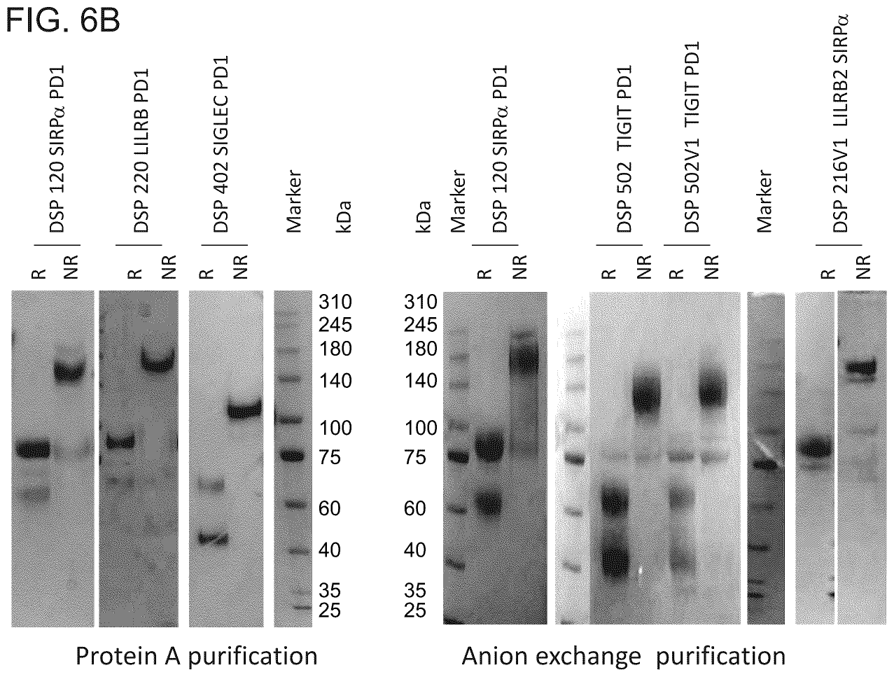

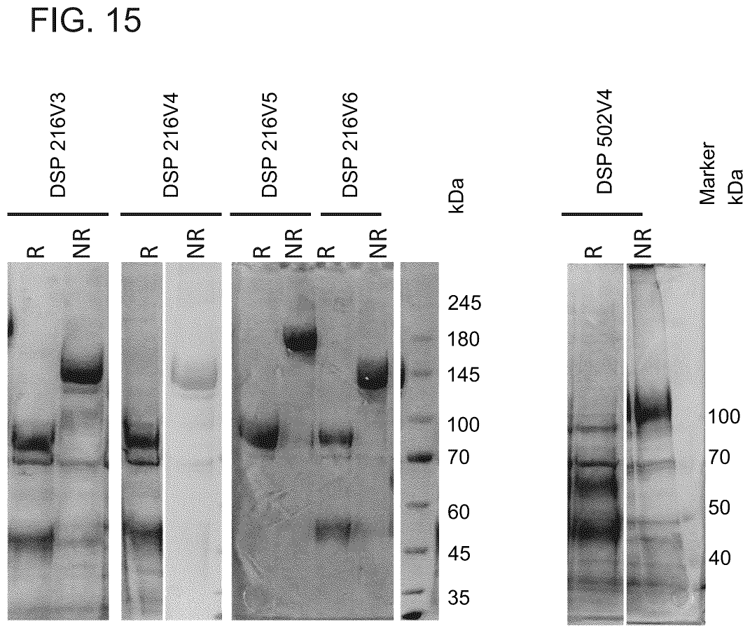

Following, several heterodimers were produced and analyzed. As demonstrated in FIGS. 6A-B and FIG. 15, a high proportion of protein of the expected heterodimer molecular weight form was observed under non-reducing conditions and the expression of the two subunits was confirmed in reducing conditions. Only a minor level of homodimers (dimers comprising two “knob” or two “hole” fragments) was detected by the SDS-PAGE.

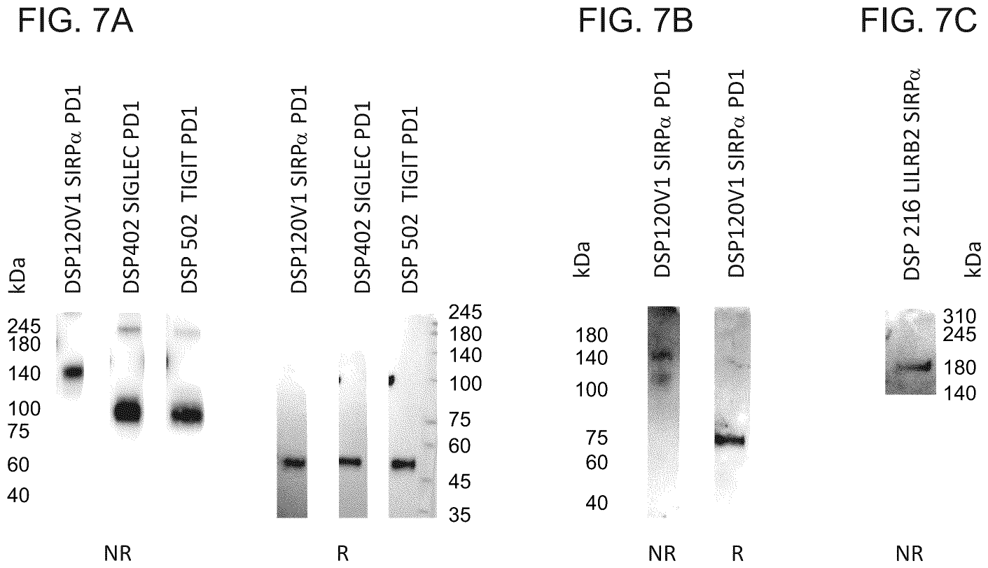

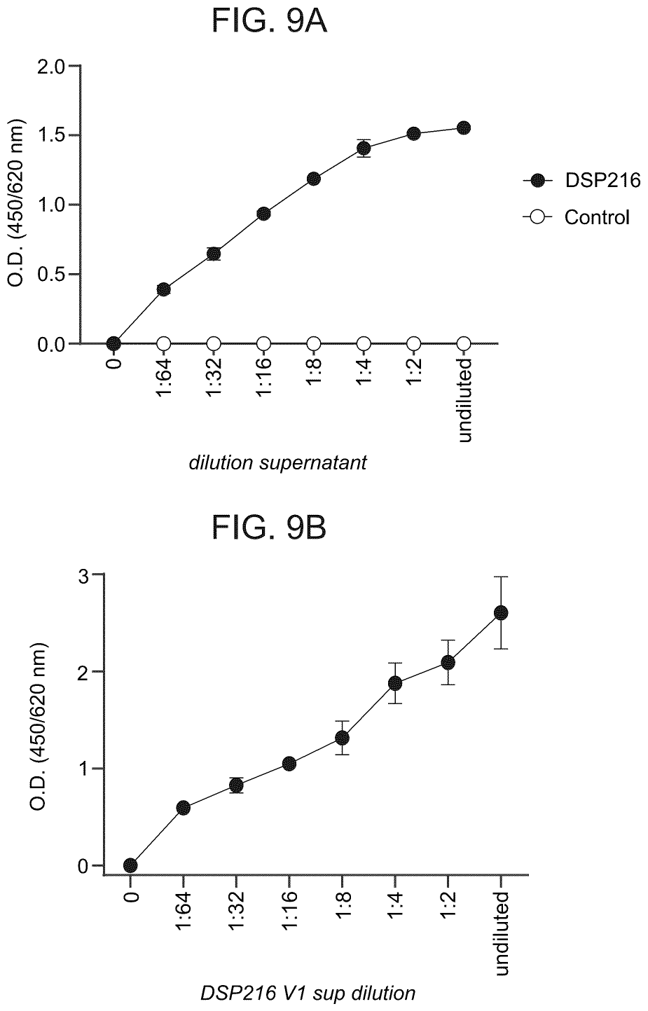

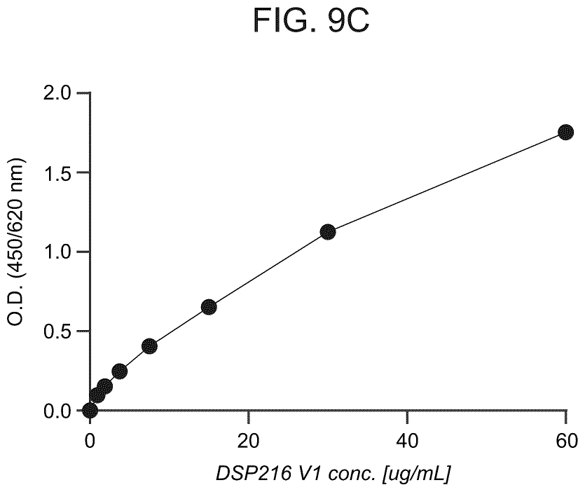

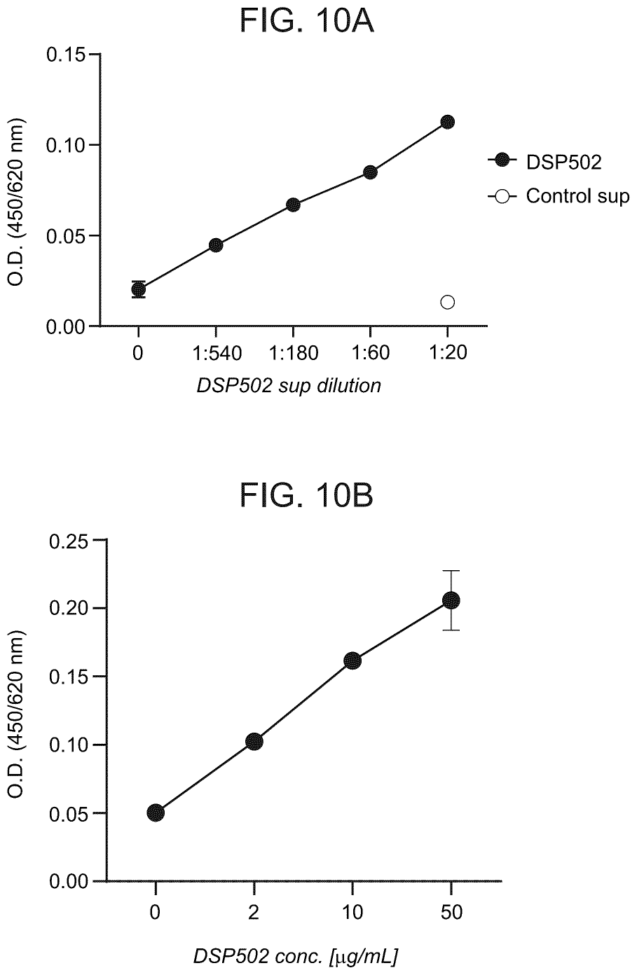

Further, the produced heterodimers contain all their designed domains; for example, DSP120V1 contains both PD1 and SIRPα domains (FIGS. 7A-B and 8A-B); DSP216 and DSP216V1 contain both LILRB2 and SIRPα domains (FIGS. 7C and 9A-C); DSP402 contains PD1 domain (FIG. 7A); DSP502 contains both PD1 and TIGIT domains (FIGS. 7A and 10A-B).

In the next step, the produced heterodimers are further analyzed according to Examples 2-8 hereinbelow according to their composition.

### Example 2

### The Heterodimers Bind their Counterpart Ligands/Receptors

### Binding Analysis of SIRPα-PD1 Heterodimers to CD47 and PDL1 Expressed on Cell's Surface

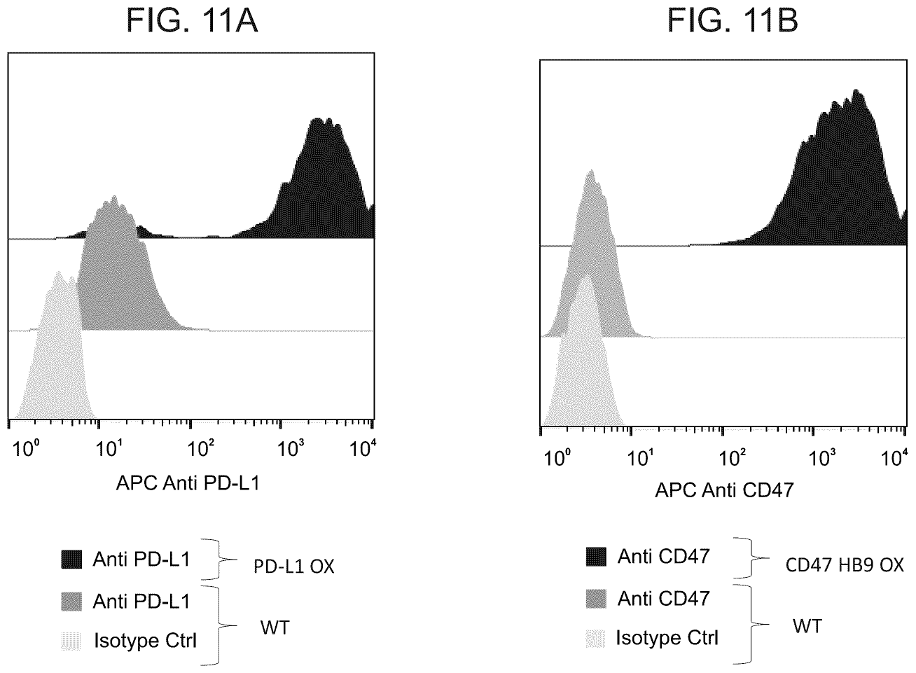

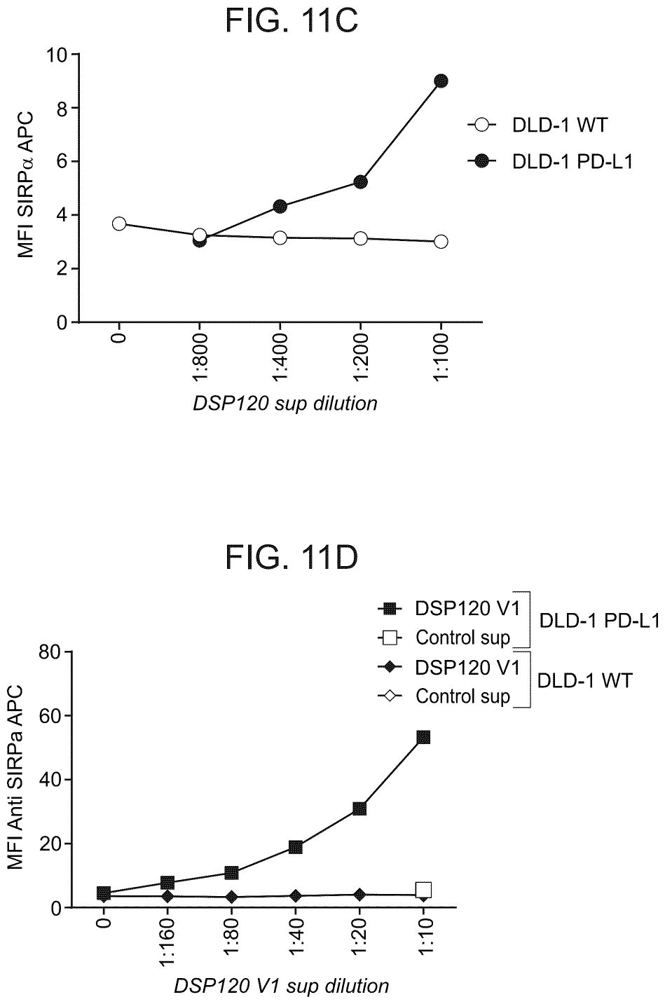

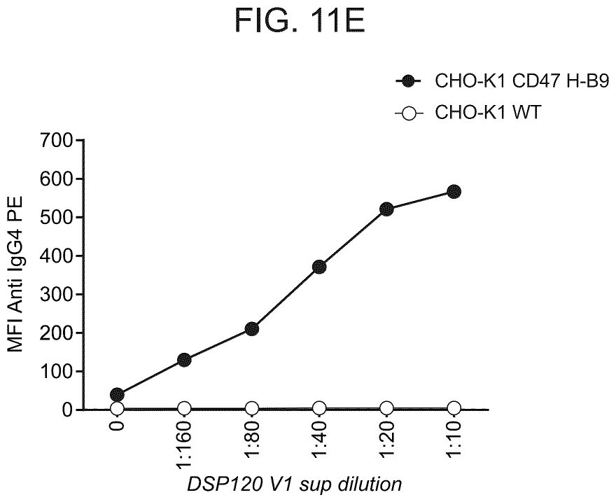

Binding of the PD1 domain of SIRPα-PD1 heterodimers referred to herein as “DSP120” and “DSP120V1” to human PDL1 expressed on cells was determined using a DLD1-PDL1 cell line overexpressing PDL1 (FIG. 11A) with anti-SIRPα as a detector antibody. DLD1-WT cells, which express low levels of endogenous PDL1 (FIG. 11A) served as a control. As shown in FIGS. 11C-D, DSP120 and DSP120V1 bound DLD1 PDL1 overexpressing cells in a dose dependent manner.

Binding of the SIRPα domain of the SIRPα-PD1 heterodimer DSP120 to human CD47 expressed on cells was determined using a CHO-K1 cell line overexpressing human CD47 (FIG. 11B) with a PE conjugated anti-IgG4 antibody as the detector antibody. CHO-K1 cells served as a control as they do not express human CD47 (FIG. 11B). As shown in FIG. 11E, DSP120 bound CHO-K1 CD47 overexpressing cells in a dose dependent manner.

### Binding Analysis of SIRPα-PD1 Heterodimers to Plate Bound CD47 or PDL1

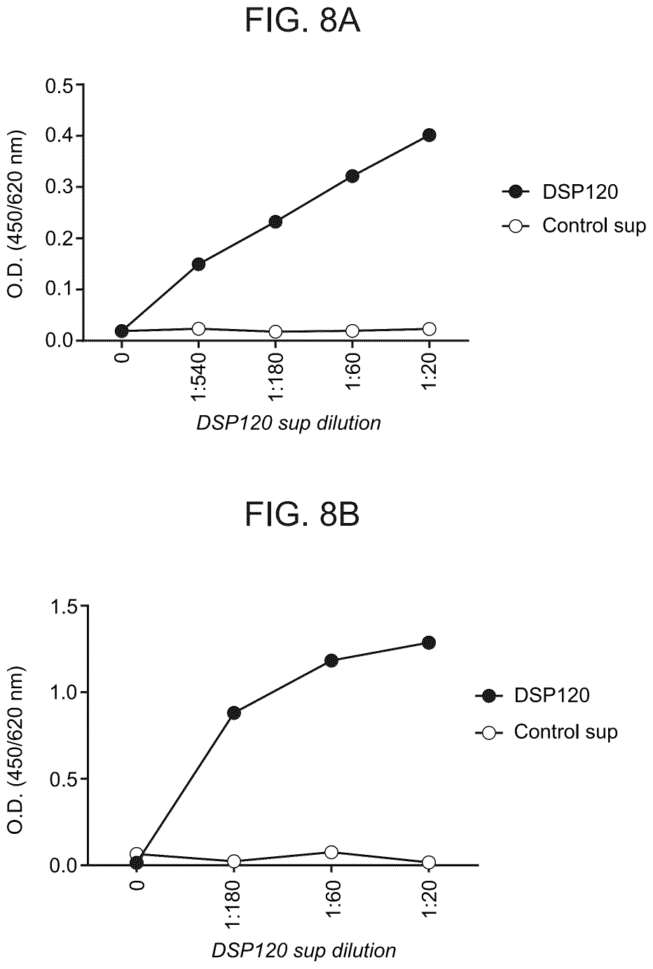

Binding of the PD1 and SIRPα domains of the SIRPα-PD1 heterodimer DSP120 to plate bound PDL1 or CD47 was determined following incubation using an anti-human PD-1 (For CD47 coated plate) or anti-human SIRPα antibody (for PDL1 coated plate), followed by incubation with a corresponding HRP conjugated secondary antibody. Detection was effected with a TMB substrate. FIG. 8A shows binding of DSP120 to CD47-coated plates in a concentration dependent manner and FIG. 8B demonstrates binding of DSP120 to PDL1-coated plates in a concentration dependent manner.

### Binding Analysis of SIRPα-LILRB2 Heterodimers to CD47 and HLA-G Expressed on Cell's Surface

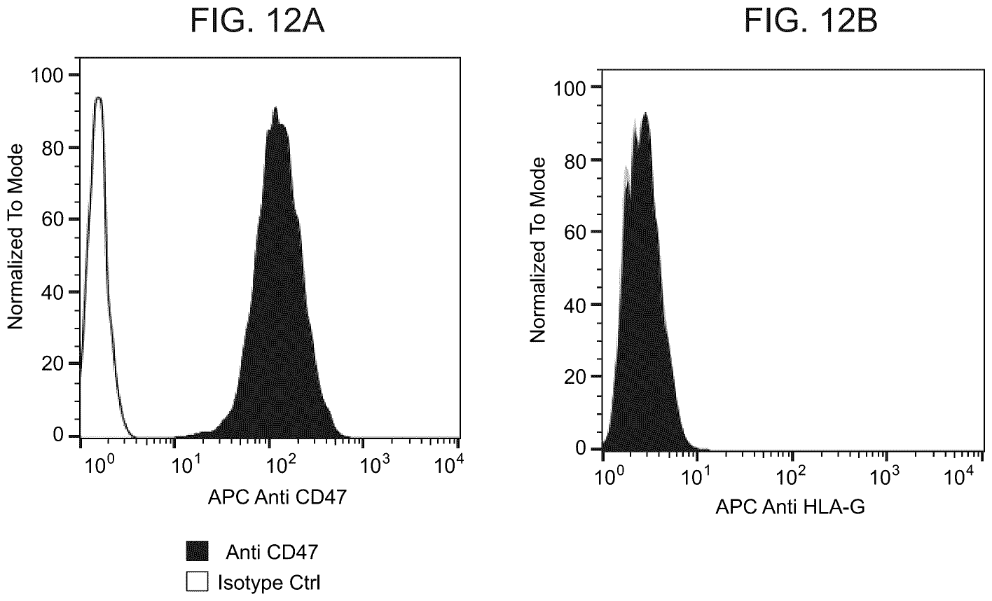

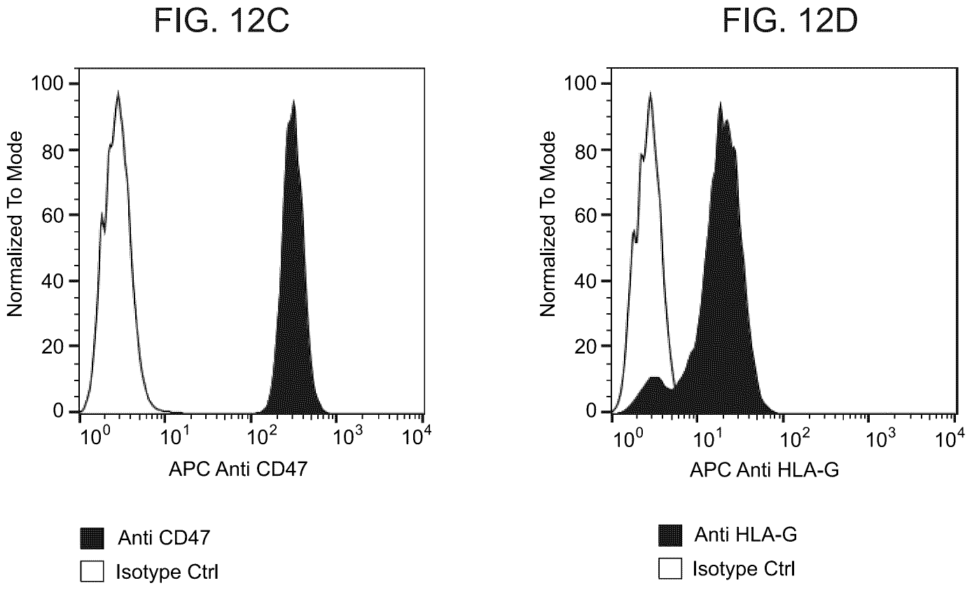

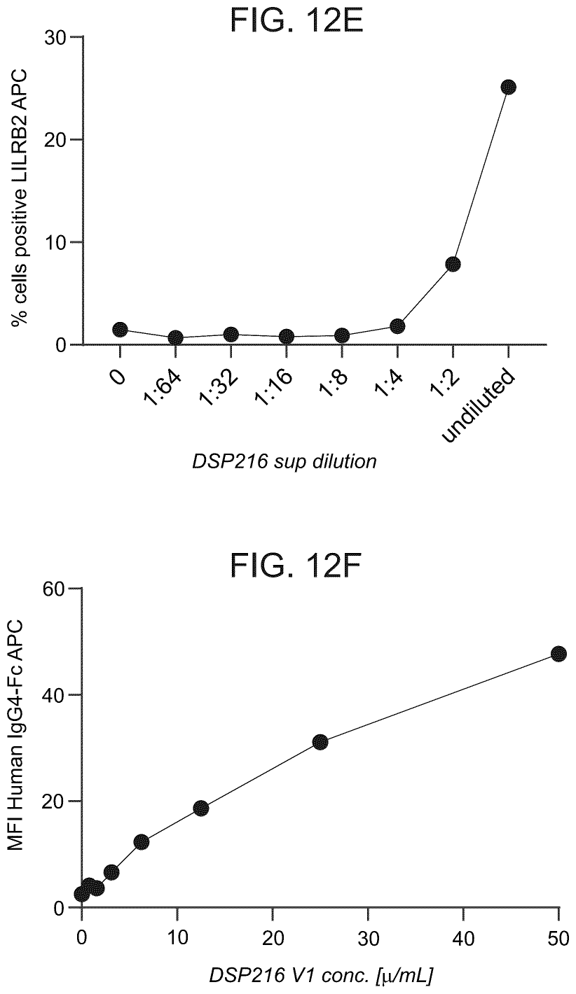

Binding of the SIRPα domain of the SIRPα-LILRB2 heterodimer referred to herein as “DSP216” to human CD47 expressed on cells was determined using a HT1080 cell line expressing human CD47 (FIG. 12A) and not expressing HLA-G (FIG. 12B) and an APC conjugated anti-LILRB2 as a detector antibody. As shown in FIG. 12E, DSP216 bound to CD47 expressing cells in a dose dependent manner.

Binding of the SIRPα and LILRB2 domains of the SIRPα-LILRB2 heterodimer referred to herein as “DSP216V1” to HLA-G and/or human CD47 expressed on cells was determined using a HT1080 cell line overexpressing HLA-G (FIG. 12D) and an anti-human IgG4 antibody. As shown in FIG. 12F, DSP216V1 bound to HLA-G and human CD47 expressing cells in a dose dependent manner.

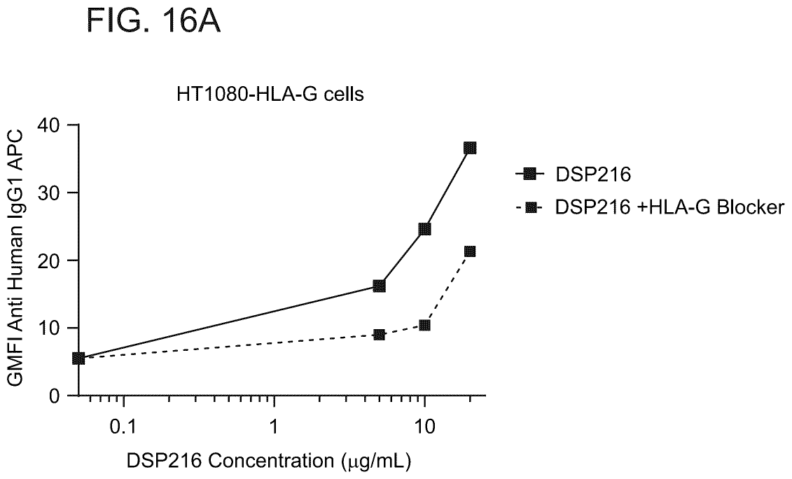

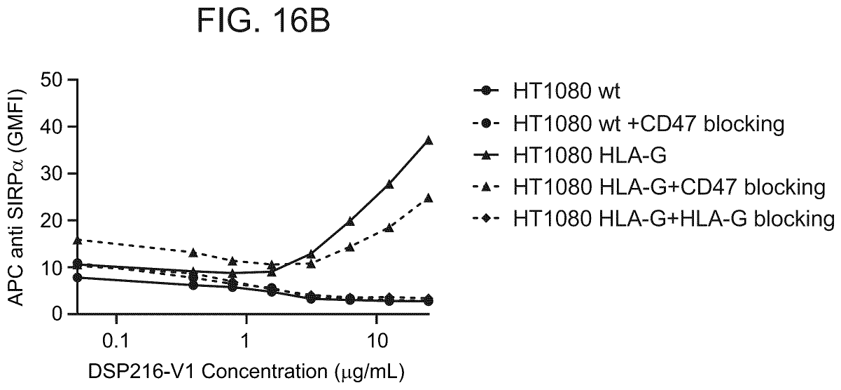

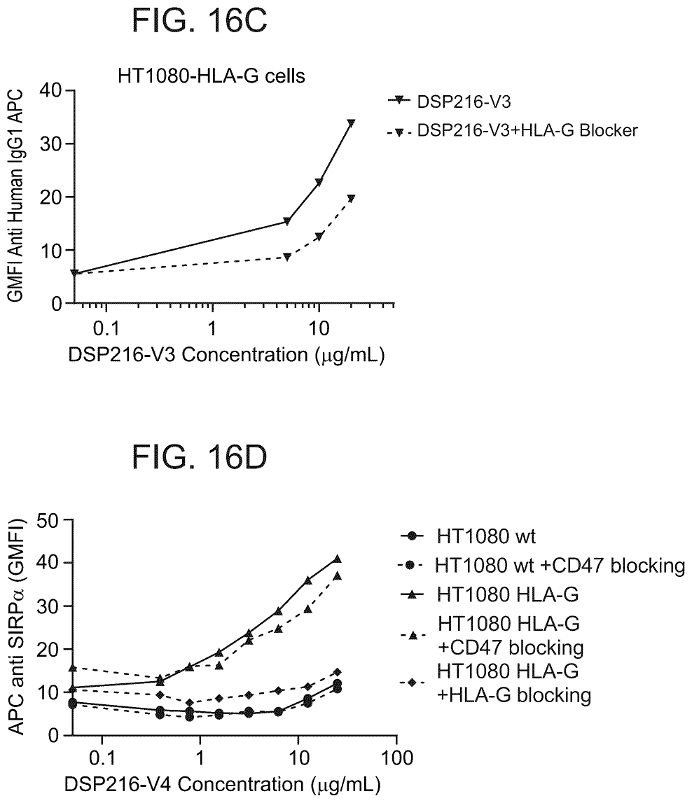

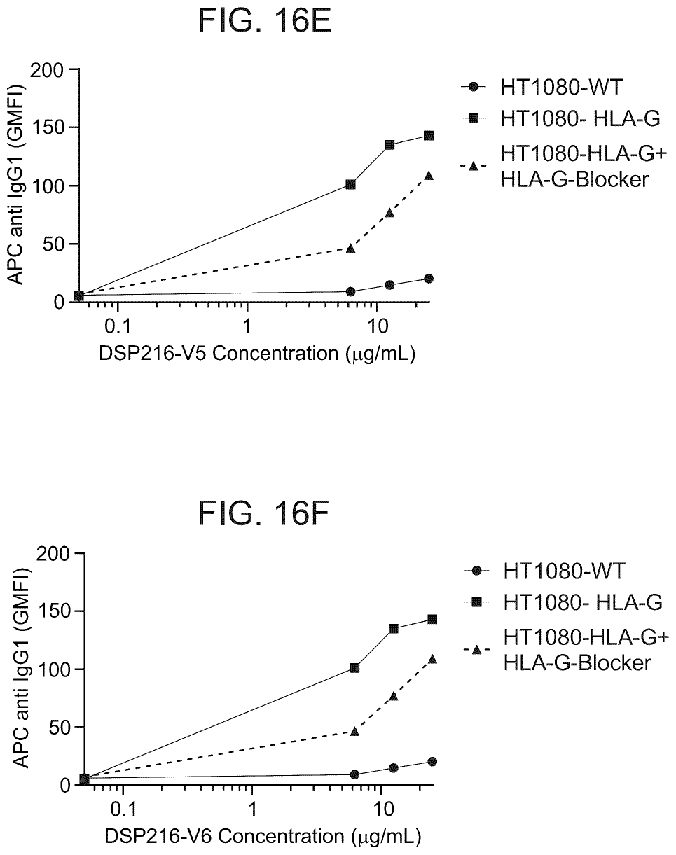

As HT1080-HLA-G express both HLA-G and CD47, the specific binding to human CD47 and HLA-G was further tested using blocking antibodies against each one of the ligands. As shown in FIG. 16A, specific dose dependent binding of DSP216 to HLA-G expressed on the HT1080-HLA-G cell line was confirmed using anti human HLA-G blocking antibody (an APC conjugated anti-human IgG1 antibody was used as a detector antibody). Similarly, the specific dose dependent binding, of DSP216V1 to human CD47 on the HT1080 cell line as well as to human CD47 and human HLA-G expressed on the HT1080-HLA-G cell line was confirmed using an anti-human CD47 and anti-human HLA-G blocking antibodies (FIG. 16B, an APC conjugated anti-SIRPα antibody was used as a detector antibody).

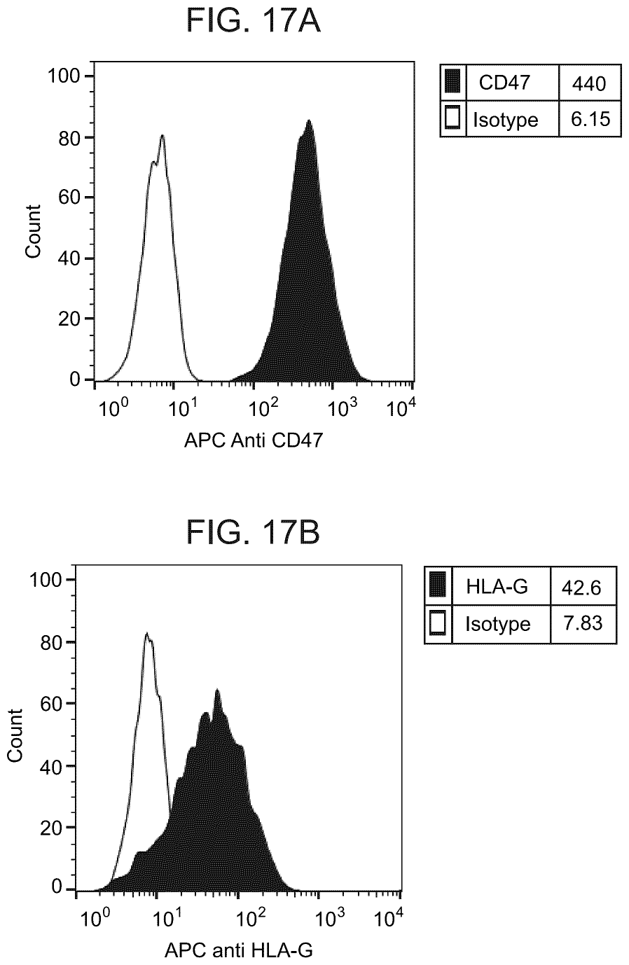

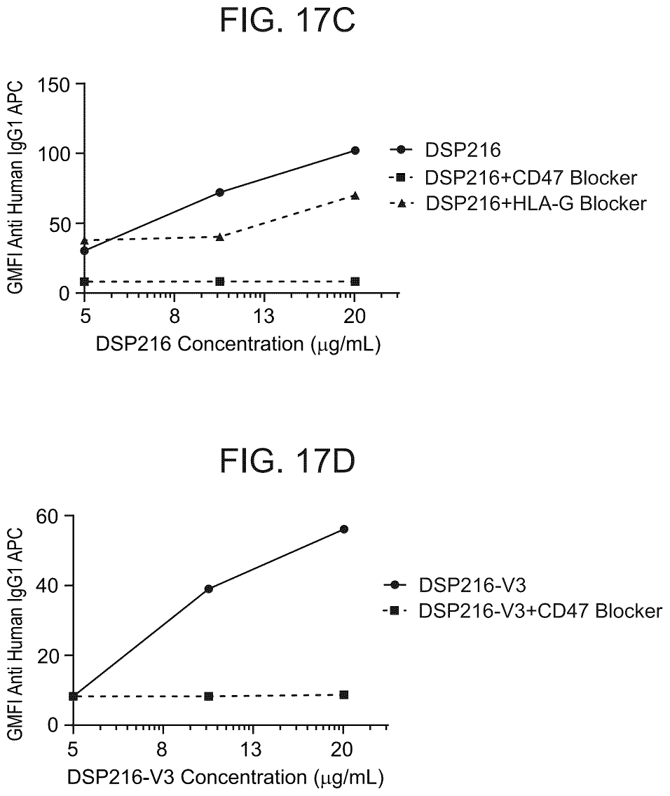

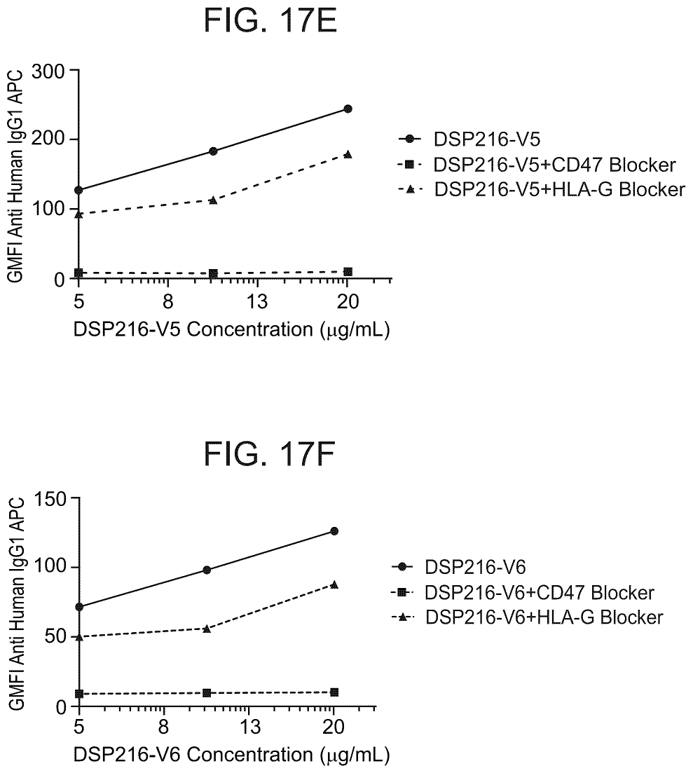

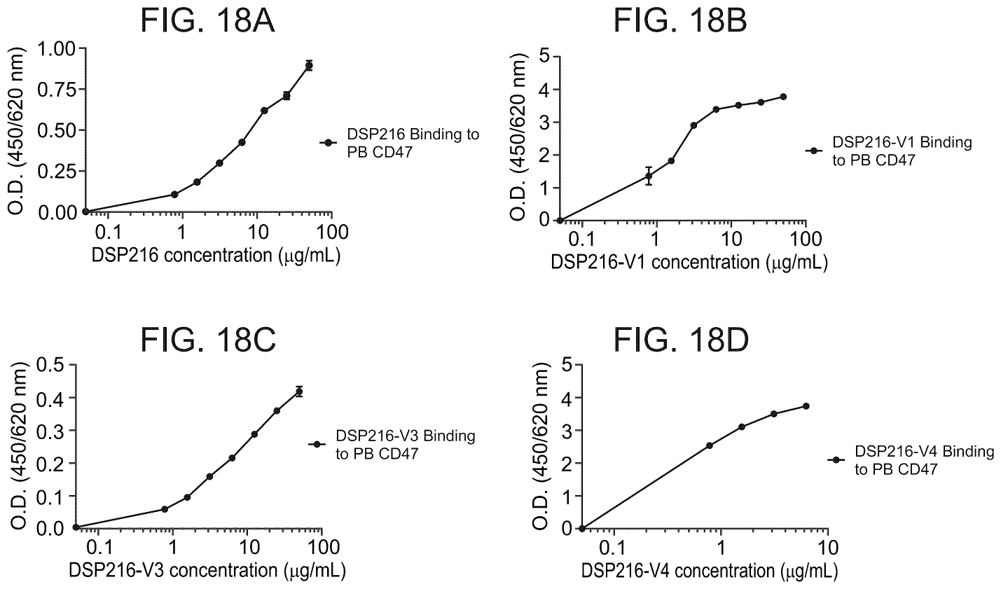

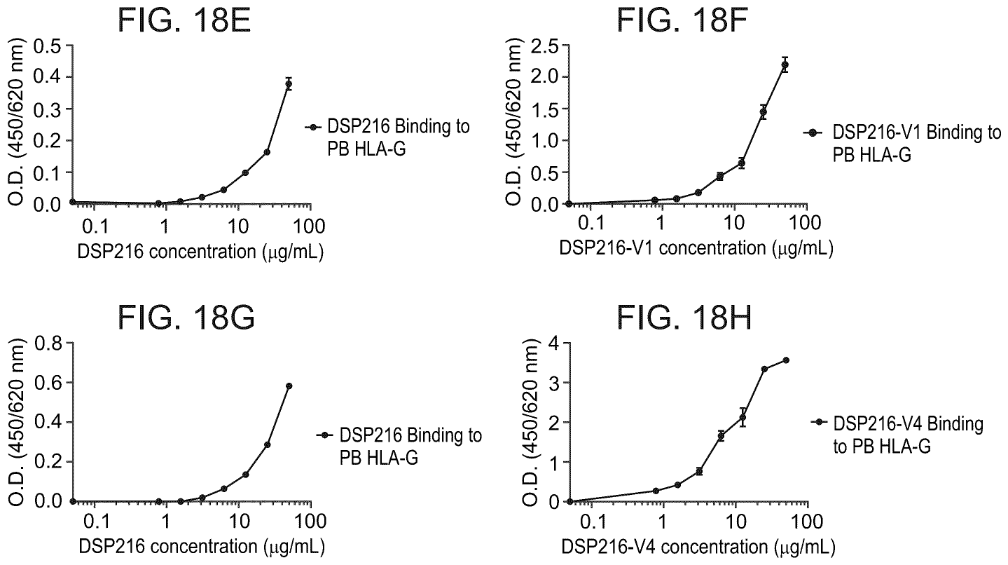

In the same manner, specific dose dependent binding of the LILRB2 domain to human HLA-G and the specific dose dependent binding of the SIRPα domain to CD47 of other SIRPα-LILRB2 heterodimers referred to herein as “DSP216V3”, “DSP216V4”, “DSP216V5” and “DSP216V6” were determined using a HT1080-HLA-G cell line overexpressing HLA-G or JEG3 cell line (both lines expressing both human CD47 and human HLA-G) using an anti-human HLA-G blocking antibody or an anti-human CD47 blocking antibody (FIGS. 16C-17F, an APC conjugated anti-human IgG1 or an APC conjugated anti-SIRPα antibody was used as a detector antibody).

### Binding Analysis of SIRPα-LILRB2 Heterodimers to Plate Bound Human CD47 or Human HLA-G

Binding of the SIRPα and LILRB2 domains of the SIRPα-LILRB2 heterodimers DSP216, DSP216V1, DSP216V3 and DSP216V4 to plate bound CD47 or HLA-G was determined following incubation using an anti-human SIRPα antibody (for HLA-G coated plate) followed by incubation with a corresponding HRP conjugated secondary antibody; or using an anti-human anti-IgG1-HRP or anti-human IgG4-HRP. Detection was effected with a TMB substrate. As shown in FIGS. 9A-C and 18A-H, all tested heterodimers bound both plate bound CD47 and plate bout HLA-G in a concentration dependent manner.

### Binding Analysis of SIGLEC-10-PD1 Heterodimers to CD24 and PDL1 Expressed on Cell's Surface

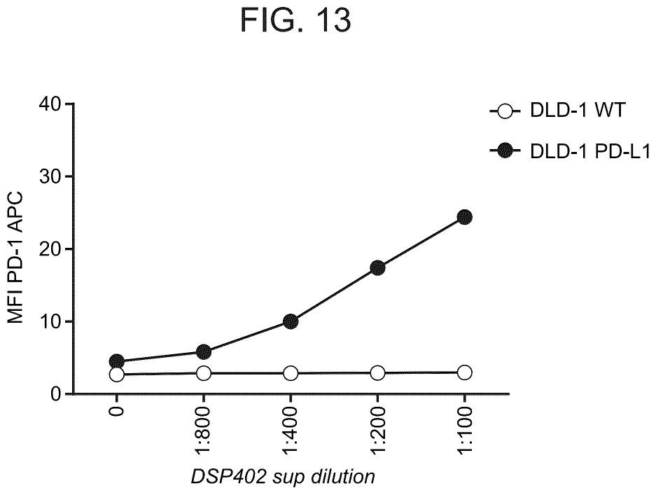

Binding of the PD1 domain of the SIGLEC-10-PD1 heterodimer referred to herein as “DSP402” to human PDL1 was determined using DLD1-PDL1 cell line overexpressing PDL1 and an APC conjugated anti-PD1 as a detector antibody (FIG. 11A). DLD1-WT cells, which express low levels of endogenous PDL1, served as a control (FIG. 11A). As shown in FIG. 13, DSP402 bound to DLD1 PDL1 overexpressing cells in a dose dependent manner.

Binding of the SIGLEC-10 domain to its receptor is tested using CD24 expressing cells.

### Binding Analysis of TIGIT-PD1 Heterodimers to PVR, PDL1 and FcγRIIIa (CD16) Expressed on Cell's Surface

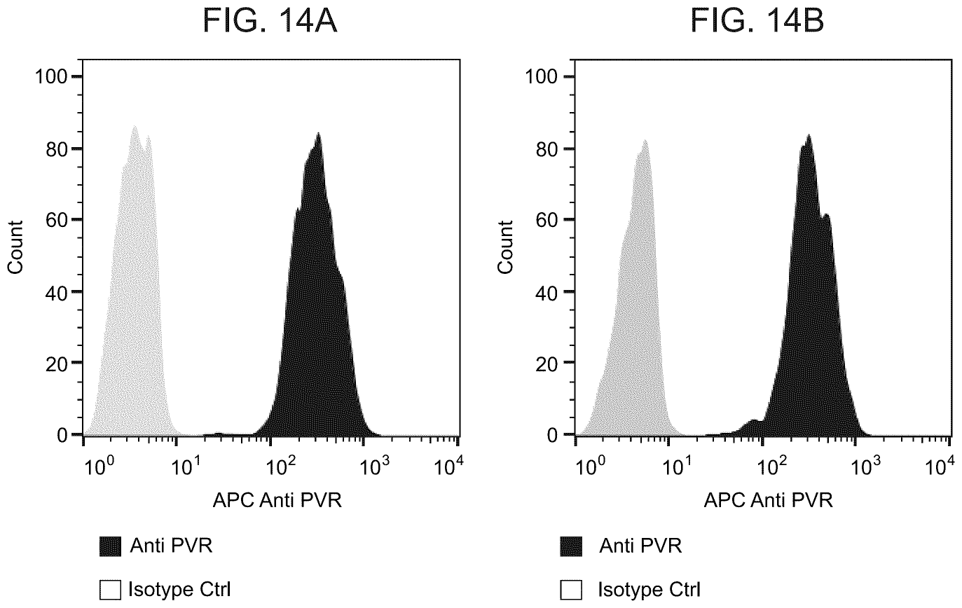

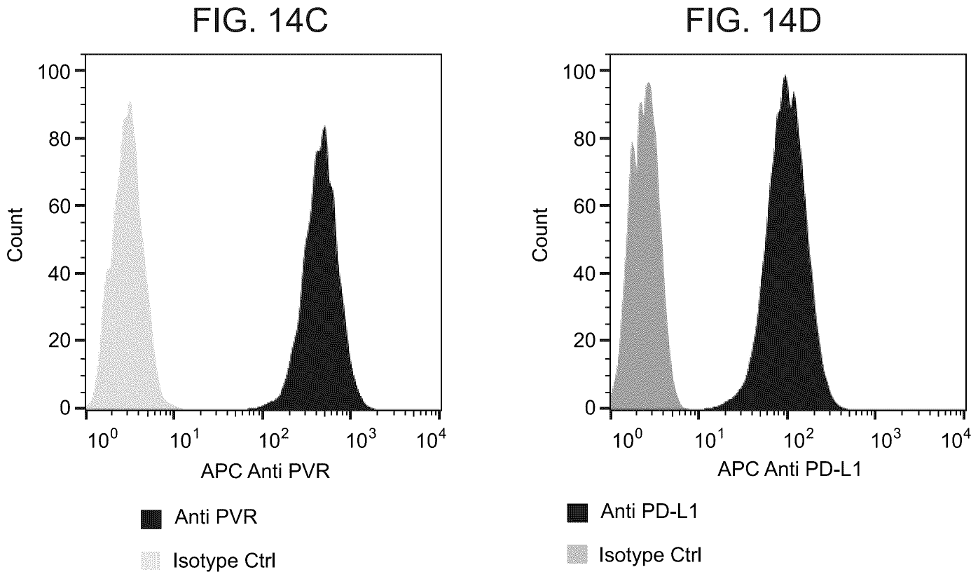

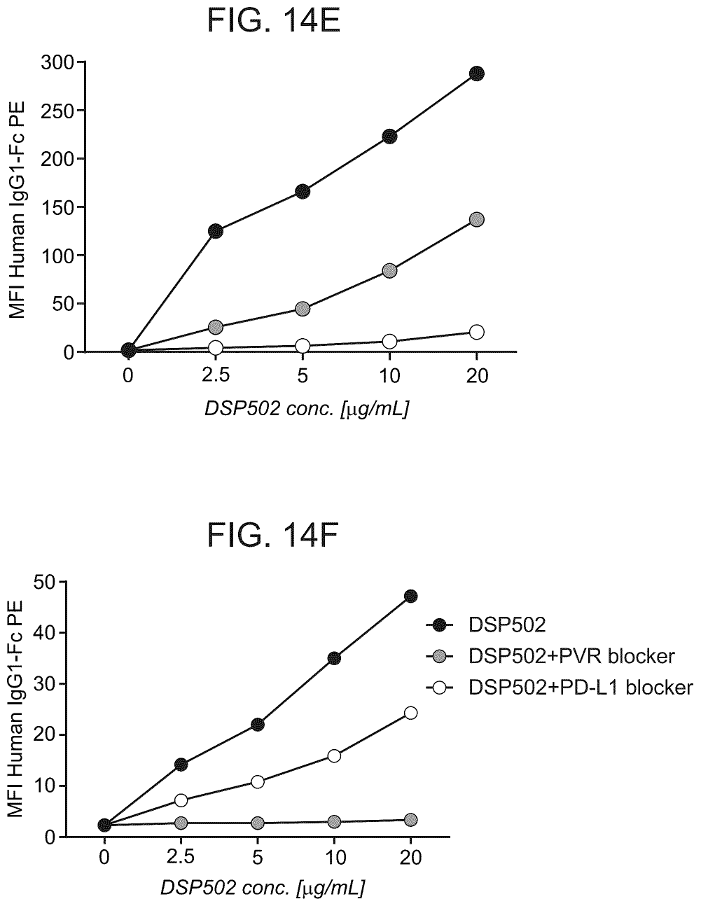

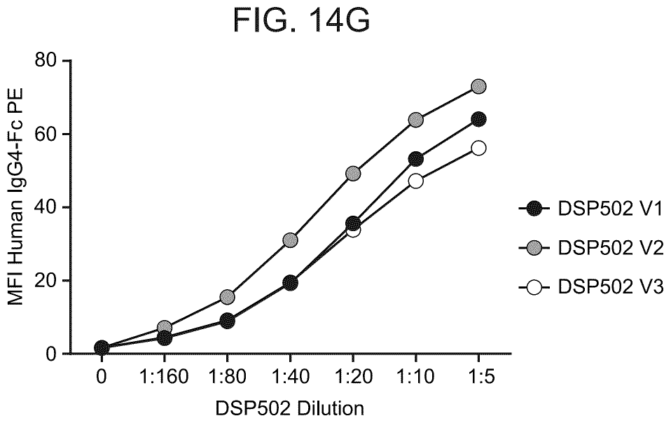

Binding of the PD1 domain of the TIGIT-PD1 heterodimer referred to herein as “DSP502” to human PDL1 was determined using a DLD1-PDL1 cell line overexpressing PDL1 (FIG. 11A), as well as a HT1080 cell line endogenously expressing PD-L1 (FIG. 14D). Binding of the TIGIT domain of DSP502 to human PVR was determined using DLD1-PDL1 and HT1080 cell lines, both expressing high levels of endogenic PVR (14B and 14C). PE conjugated anti-human IgG1 antibody was used as a detector antibody in both assays.

As shown in FIGS. 14E-F, DSP502 bound PDL1 expressing cells in a dose dependent manner and the binding was blocked by an anti-human PD-L1 blocker antibody.

FIGS. 14E-F also demonstrate dose dependent binding of the TIGIT domain of DSP502 to the PVR expressing cells, as indicated by blocking of DSP502 binding to these cells using an anti-human PVR blocking antibody.

FIG. 14G demonstrates dose dependent binding of the TIGIT domain of other TIGIT-PD1 heterodimers (referred to herein as “DSP502V1”, “DSP502V2” and “DSP502V3”) to the DLD-1 WT cells which express PVR and not PD1 (FIGS. 14A and 11A). PE conjugated anti-human IgG4 antibody was used as a detector antibody. The pattern of binding of these three proteins is similar.

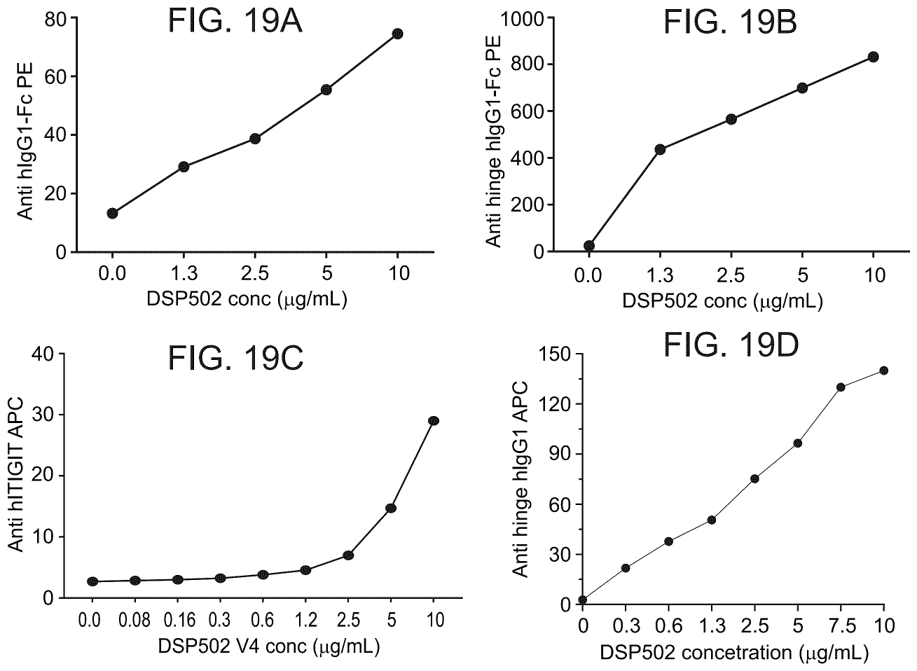

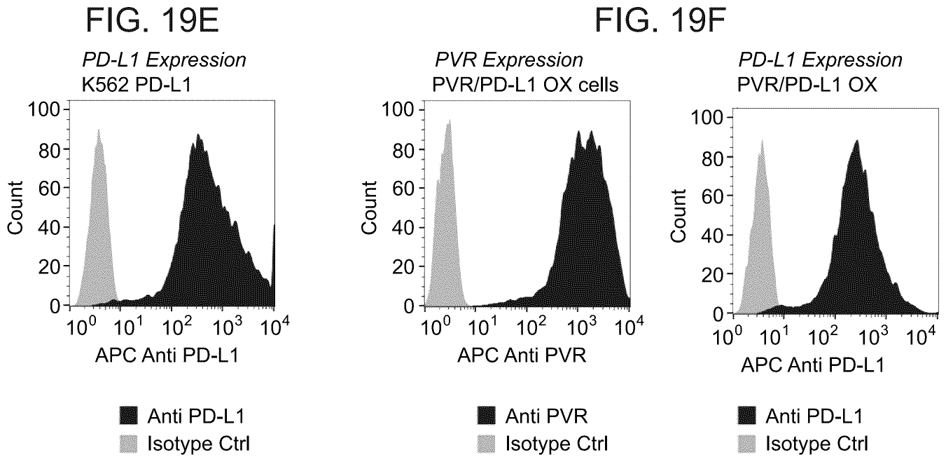

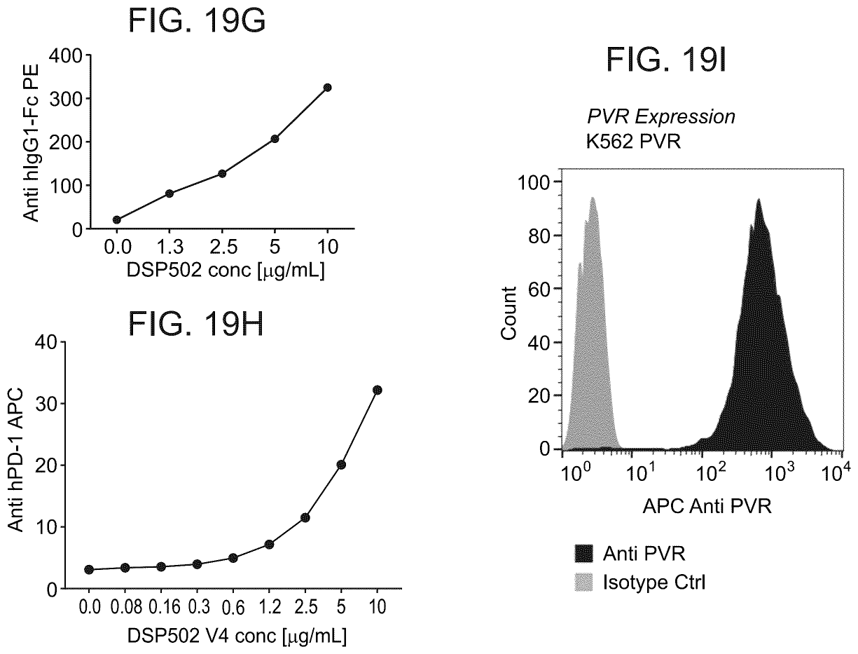

In addition, dose dependent binding of the PD1 and TIGIT domains of DSP502 and another TIGIT-PD1 heterodimer referred to herein as “DSP502V4” (which contains a LALA mutation on the Fc-IgG1 domains) to human PDL1 and human PVR, respectively, were determined using a K562 PD-L1 cell line overexpressing PD-L1, a K562 cell line overexpressing PVR, as well as K562 PVR/PD-L1 cell line overexpressing both PVR and PD-L1 (FIGS. 19A-I).

A PE conjugated anti-human IgG1 antibody or APC conjugated anti-TIGIT and anti-human IgG1 antibodies were used as a detector antibodies in the binding assays, as indicated in the Figures.

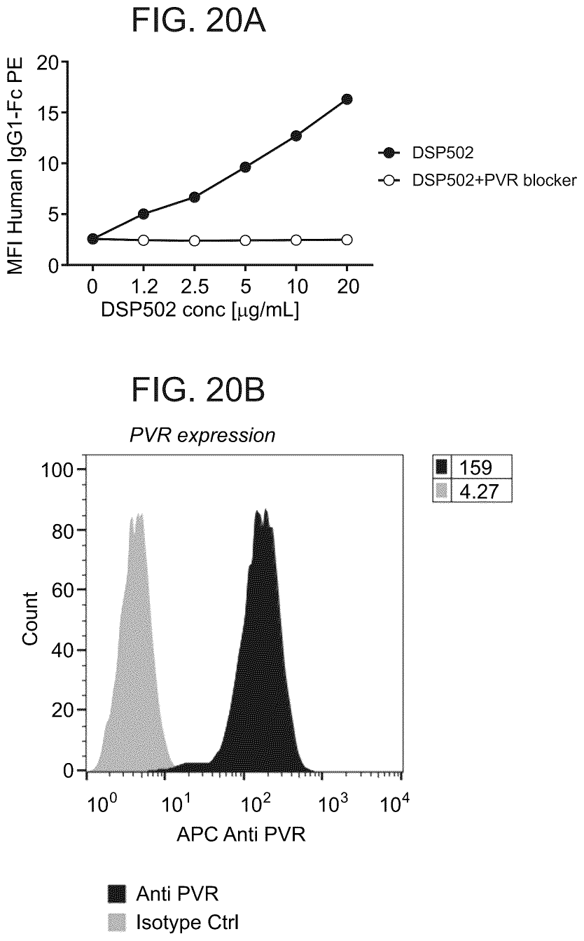

Dose dependent binding of the TIGIT domain of DSP502 to SKOV3 cell line, expressing high levels of endogenic PVR was also observed (FIGS. 20A-B, a PE conjugated anti-human IgG1 antibody was used as a detector antibody).

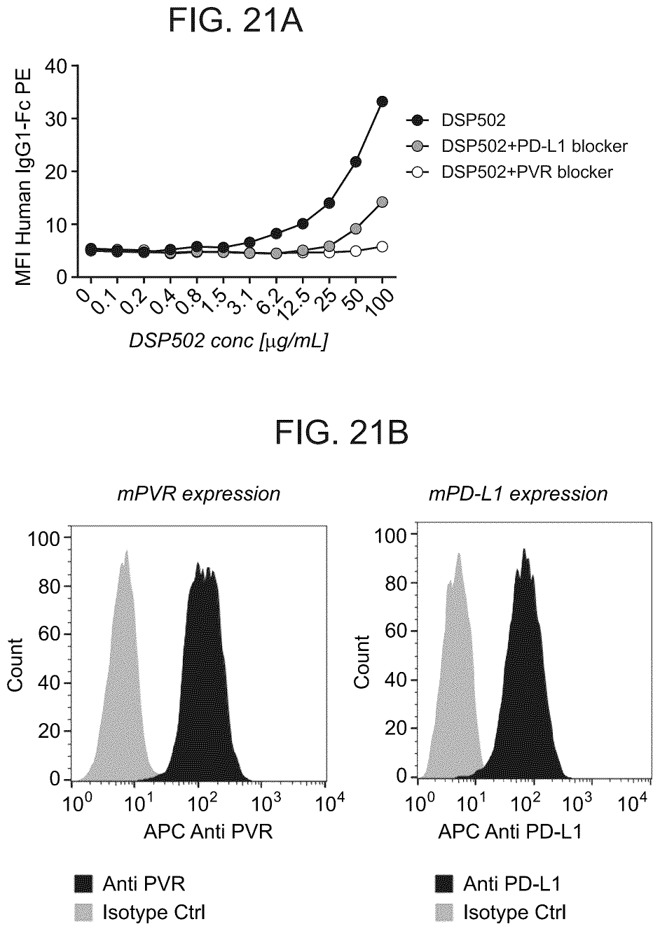

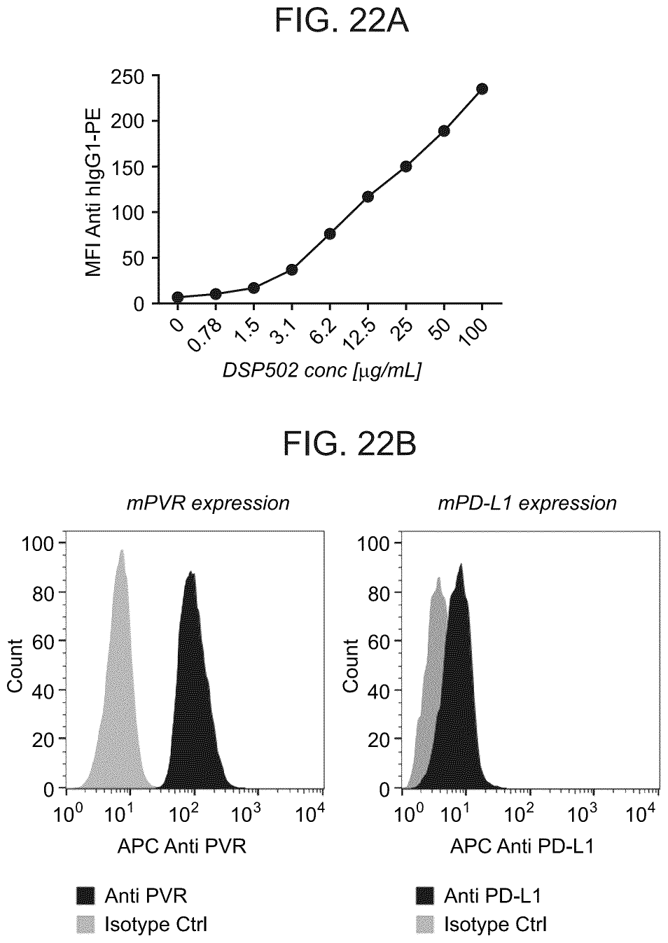

Dose dependent binding of the PD1 and TIGIT domains of DSP502 to mouse PDL1 and mouse PVR expressed on cells was also observed, using a AB12 cell line endogenously expressing PVR (FIGS. 22A-B, a PE conjugated anti-human IgG1 antibody was used as a detector antibody) or Renca, cell line endogenously expressing both PD-L1 and PVR (FIGS. 21A-B, a PE conjugated anti-human IgG1 antibody was used as a detector antibody). The specific binding of DSP502 to each of the ligands (PDL1 and PVR) expressed on Renca cells was demonstrated using anti-mPDL1 and anti-mPVR blocker-antibodies.

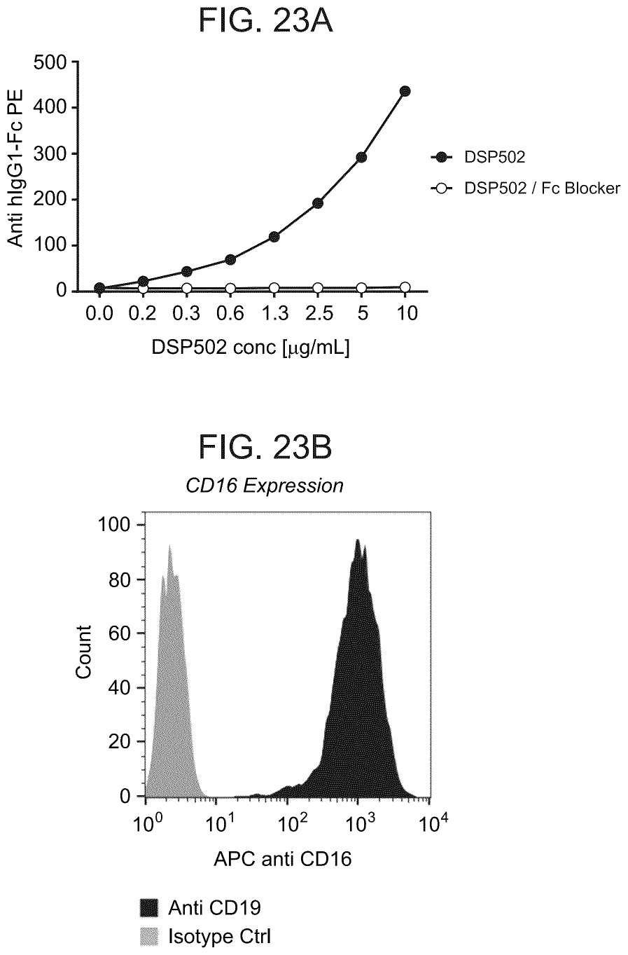

Further, binding of the Fc domains of DSP502 to the human Fc receptor CD16 was determined using a Jurkat NFAT cell line overexpressing CD16 (FIG. 23B). A PE conjugated anti-human IgG1 antibody was used as a detector antibody. As shown in FIG. 23A, DSP502 bound the cells in a dose dependent manner. The specificity of the binding to CD16 was demonstrated using an Fc blocker which completely blocked binding of the heterodimer to the cells.

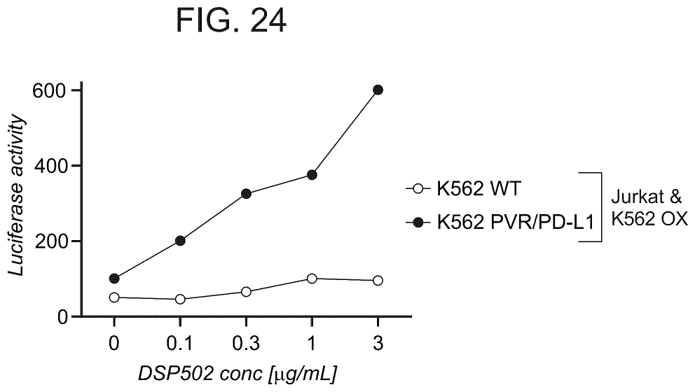

To further test the effect of binding of the heterodimer to the Fc receptor, a co-culture assay of K562 PVR/PDL-1 cells (Target cells) with Jurkat NFAT cells overexpressing CD16 (Effector cells), followed by analysis of the luciferase signal following 6 hours incubation. The Jurkat-NFAT-CD16 cells stably express the Lucia luciferase reporter gene under the control of an ISG54 promoter fused to NFAT elements. FcγRIIIa binding was measured as a bioluminescent signal produced by the luciferase upon the addition of the detection reagent. As shown in FIG. 24, co-culturing the cells in the presence of DSP502 increased luciferase secretion. Importantly, luciferase secretion was not detected when the Jurkat NFAT-CD16 cells were incubated as a single-culture assay (i.e. in the absence of K562 PVR/PDL-1 cells) with DSP502. Hence, binding of DSP502 to FcγRIIIa expressed on the Jurkat cells, when it is not anchored to PVR and/or PDL1, is not sufficient for initiation of signal transduction.

### Binding Analysis of TIGIT-PD1 Heterodimers to Plate Bound PVR

Binding of the TIGIT domain of the TIGIT-PD1 heterodimer DSP502 to plate bound PVR was determined following incubation using an anti-human PD1 antibody followed by incubation with a corresponding HRP conjugated secondary antibody. Detection was effected with a TMB substrate. As shown in FIGS. 10A-B, DSP502 bound plate bound PVR in a concentration dependent manner.

### Example 3A

### The Heterodimers Bind their Counterpart Ligands/Receptors Simultaneously, as Determined by ELISA

Binding of both sides of heterodimers to their counterparts, i.e., the binding of PD1 to PDL1, LILRB2 to HLA-G, SIRPα to CD47, TIGIT to PVR and SIGLEC-10 to CD24 is tested by a sandwich ELISA based assay. This assay is also used to compare the functional properties of different variants of the heterodimer proteins.

Flat bottom 96-wells plates are pre-coated with receptor/ligand of one arm by incubating with a recombinant counterpart protein such as CD47 protein, PDL1 protein, HLA-G protein, PVR or CD24, followed by blocking and washing. Serially diluted cell culture supernatant or purified protein samples containing the produced heterodimers are added to the corresponding pre-coated wells. Following an additional washing step, biotinylated or an unlabeled soluble receptor/ligand against the second arm of the heterodimer is added and allowed to bind to the captured protein. Thereafter streptavidin-HRP or HRP conjugated Ab against the second receptor/ligand is added and detection is effected with a TMB substrate according to standard ELISA protocol using a plate reader (Thermo Scientific, Multiscan FC) at 450 nm, with reference at 620 nm.

Alternatively, plates are coated with a mix of two proteins at equal-molar quantity and binding is detected with an IgG specific antibody.

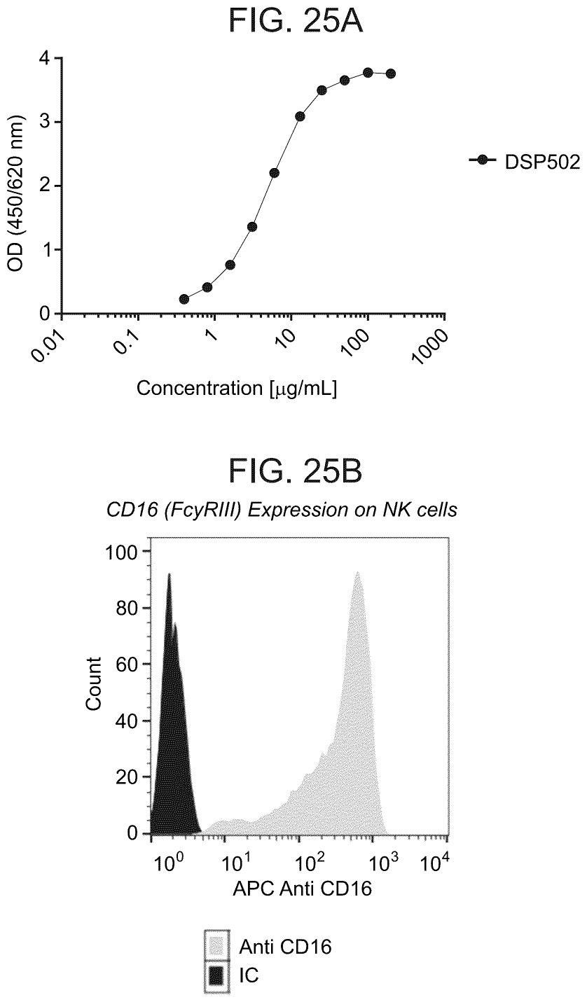

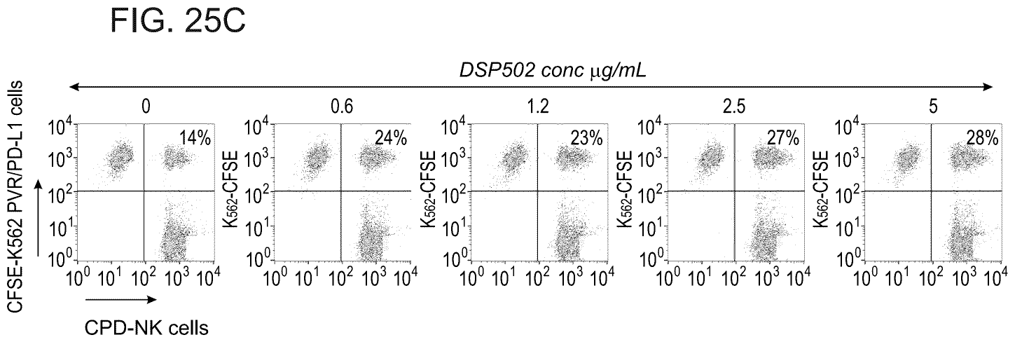

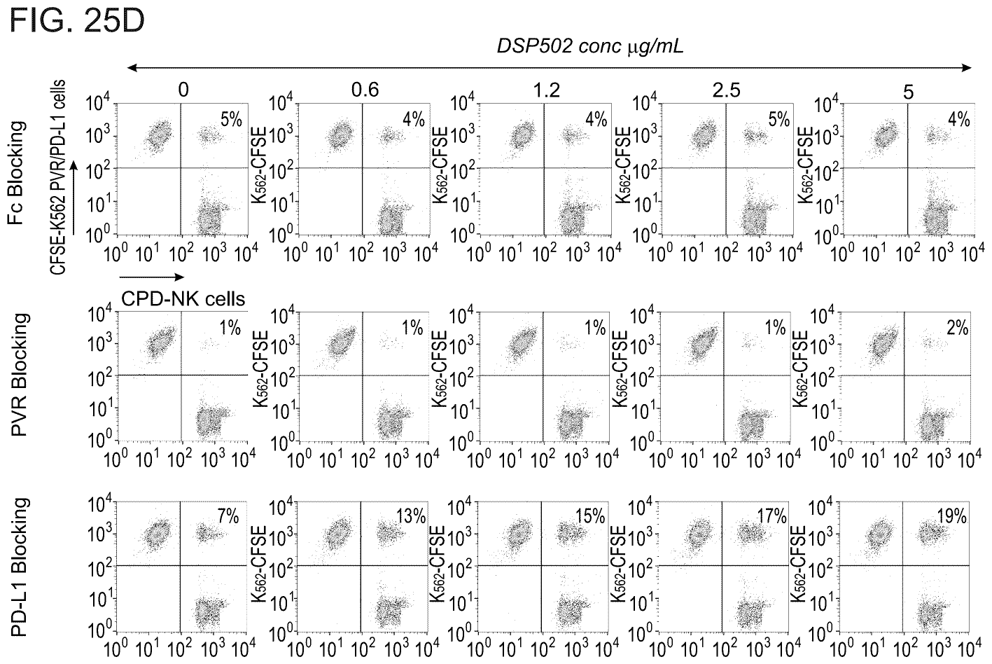

As shown in FIG. 25A, the TIGIG-PD1 heterodimer DSP502 bound both PVR and PDL1 simultaneously.

### Example 3B

### The Heterodimers Bind their Counterpart Ligands/Receptors Simultaneously, as Determined by Flow Cytometry

Simultaneously binding of the TIGIT-PD1 heterodimer DSP502 to all its counterparts (i.e. e.g., binding of IgG1-Fc to FcγRIIIa, TIGIT to PVR and PD1 to PDL1 was tested by flow cytometry, using NK primary cells expressing FcγRIIIa (FIG. 25B) and K562 PVR/PD-L1 expressing PVR and PDL1 (FIG. 19F). Specifically, NK cells pre-labeled with a CPD dye were co-cultured with K562 PVR/PD-L1 cells pre-labeled with a CFSE dye, in a ratio of 2: 1, respectively, in the presence of different concentrations of DSP502. The formation of doublets was analyzed using flow cytometry and appeared as double stained events. As shown in FIG. 25C, DSP502 mediated doublets formation, indicating binding of the DSP simultaneously to both cell types. Further, these doublets forming mediation-activity by DSP502 was blocked by specific blocker antibodies to PVR, PD-L1 or FcγRIII (FIG. 25D), indicating that the optimal conditions for doublets formation are when the three receptors are involved.

### Example 4

### The Effect of the Heterodimers on Blocking Ligand—Receptor Binding

The heterodimers are designed to block the interaction of endogenous ligand/receptor expressed on target cells with the native receptor/ligand.

Thus, for example, the PD1 part of the relevant heterodimer is designed to block the interaction of endogenous PD1 expressed on T cells with PDL1 expressed on tumor cells. To this end, effectiveness of the produced heterodimers as blockers of this interaction is evaluated. Plates are coated with a recombinant human PDL1. Following, plates are washed and incubated for 1 hour with different concentrations of the produced PD1 containing heterodimer (see e.g., Table 1 hereinabove) or the positive control anti-PD1 blocker antibody. Biotinylated PD1 is added followed by additional incubation, and the plate is then washed and blotted with Streptavidin-HRP and TMB substrate according to standard ELISA protocol. Plates are analyzed using a plate reader (Thermo Scientific, Multiscan FC) at 450 nm, with reference at 620 nm.

Similarly, the blocking activity of the relevant heterodimers is studied to evaluate their effectiveness to block PVR-TIGIT, SIGLEC10-CD24, LILRB2-HLA-G, and CD47-SIRPα binding.

### Example 5

### The In-Vivo Anti-Tumor Effect of the Heterodimers

Experimental Design:

Three different in-vivo mouse models are used for testing the efficacy of the produced heterodimers in treating cancer:
1. NSG mice inoculated with human stem cells or with human PBMCs or with immobilized human PBMCs and with human tumor cells expressing the target of the heterodimer.
2. Nude-SCID mice inoculated with human tumor cells.
3. Syngeneic mouse tumor models using the surrogate mouse protein of the tested heterodimer.

In all models, mice are inoculated with tumor cells intravenously (IV), intraperitoneally (IP), subcutaneously (SC) or orthotopically. Once the tumor is palpable (˜80 mm³), mice are treated IV, IP, SC or orthotopically, with different doses and different regimens of the produced heterodimer.

Mice are followed for weights and clinical signs. Tumors are measured few times a week by a caliper; and tumor volume is calculated according to the following equation: V=length×width²/2. Mice Weight is measured routinely. Tumor growth and survival are monitored through the whole experiment.

Infiltration and sub-typing of immune cells in the tumor is tested by resecting the tumor or draining lymph nodes, digestion and immune phenotyping using specific antibodies staining and flow cytometry analysis. Additionally, or alternatively, infiltration of immune cells or necrotic grade of tumors is determined by resecting the tumors, paraffin embedding and sectioning for immunohistochemistry staining with specific antibodies.

At sacrificing, mice organs are harvested and embedded into paraffin blocks for H&E and IHC staining.

Blood samples are taken from mice at different time points, according to common procedures, for the following tests: PK analysis, cytokines measurements in plasma, FACS profiling of blood cells sub-populations in circulation, hematology testing, serum chemistry testing, anti-drug-antibody (ADA) analysis and neutralizing antibodies analysis (Nab).

### Results:

### The Anti-Tumor Effect of a TIGIT-PD1 Heterodimer in NSG Mice Harboring Human NSCLC Tumors

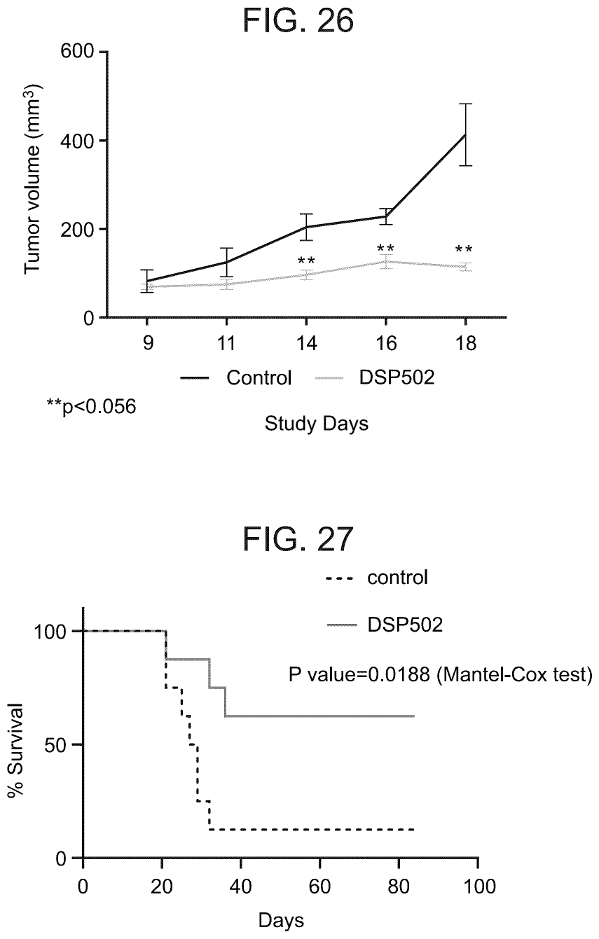

The anti-tumor in-vivo effect of the TIGIT-PD1 heterodimer DSP502 was evaluated in 11-13 weeks old male NSG mice inoculated with A549 cells, a human lung adenocarcinoma cell line. Tumor volumes in mice treated with DSP502 almost did not increase during 18 days following tumor inoculation, while the tumor volume in control mice reached a volume of 400 mm³, indicating that treatment with DSP502 resulted in a significant inhibition of tumor growth (FIG. 26).

### The Anti-Tumor Effect of a TIGIT-PD1 Heterodimer in Mice Harboring Mouse Mesothelioma Tumors

The anti-tumor in-vivo effect of the TIGIT-PD1 heterodimer DSP502 was evaluated in 7 weeks old female BALB/c mice inoculated with AB12 cells, a mouse malignant pleural mesothelioma (MPM) cell line. Treatment with DSP502 significantly prolonged mice survival, as compared to control mice (FIG. 27). For example, while 87.5% of the control treated mice died by day 33 following tumor inoculation, 62% of the DSP502 treated mice survived more than 80 days following tumor inoculation.

### Example 6

### The Effect of the LILRB2 Arm in the Relevant Heterodimers on M-CSF Dependent Macrophage Maturation

The LILRB2 arm of the heterodimers is designed to block the immunosuppressive signals induced by HLA-G expressed on tumor or immune cells towards the endogenous LILRB2 expressed on APCs such as macrophages and dendritic cells, by competing and blocking their interaction. M1-like macrophages show anti-tumor activity, while M2 macrophages have been reported to promote tumor progression. Blocking of LILRB2 with an antagonistic antibody during M-CSF dependent macrophage maturation was shown to lead to a rounder and tightly adherent M1-like (anti-tumor) phenotype with lower expression of CD14 and CD163. After stimulation of the generated macrophages with LPS, enhanced secretion of the pro-inflammatory cytokine TNFα and reduced secretion of anti-inflammatory IL-10 was detected.

To this end, the effect of the produced heterodimers comprising LILRB2 on M-CSF dependent macrophage maturation is evaluated using a flow cytometry-based detection of CD14 and CD163 and by measurement of TNFα and IL-10 release after stimulation of LPS pre-treated macrophages.

### Example 7

### The Effect of the Heterodimers Comprising a SIRPα or LILRB2 Domain on Macrophages and Polymorphonuclear Cells

The SIRPα part of the heterodimers of some embodiments is designed to block the “don't eat me” signal” induced by CD47 expressing tumor cells, towards the endogenous SIRPα expressed on APCs such as macrophages and granulocytes, by competing and blocking the interaction of CD47 on tumor cells with the endogenous SIRPα. This blockage of the “don't eat me” signal induces tumor cells phagocytosis.

The LILRB2 part of the heterodimer of some embodiments is designed in part to block the immunosuppressive signals induced by HLA-G expressed on tumor or immune cells towards the endogenous LILRB2 expressed on APCs such as macrophages and DCs, by competing and blocking the interaction of HLA-G on tumor and immune cells with the endogenous LILRB2. This blockage of the HLA-G “don't eat me signal” induces tumor cell phagocytosis and prevents the inhibitory HLA-G-LILRB2 signaling between immune cells, in turn enhancing phagocytosis.

The effect of the produced heterodimers comprising SIRPα on phagocytosis of tumor cells by human macrophages or polymorphonuclear cells (PMNs) and the effect of heterodimers comprising LILRB2 on phagocytosis of tumor cells by human macrophages or DCs are evaluated using a flow cytometry-based assay or fluorescent microscopy.

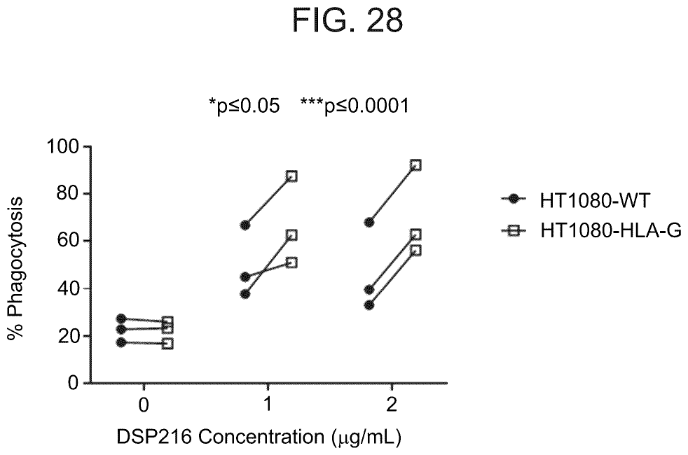

To this end, the effect of the SIRPα-LILRB2 heterodimer DSP216, on phagocytosis of tumor cells by human PMNs (granulocytes) was evaluated using a flow cytometry-based assay. Granulocytes from three different donors were incubated with 1, 2 or 5 μg/mL DSP216 and then co-cultured in a 1:1 E:T ratio with the tumor cell line HT1080 expressing human CD47 or HT1080-HLA-G expressing both human CD47 and human HLA-G (FIGS. 12A and 12D). As shown in FIG. 28, DSP216 treatment increased phagocytosis percentages of HT1080 cells and that of HT1080-HLA-G to a higher extent.

### Example 8

### NK Cells Cytotoxic Activity by the Heterodimers Comprising a TIGIT Domain

Natural killer (NK) cells induce direct cytotoxicity or secretion of cytokine/chemokine without recognizing a specific antigen as B and T cells. NK cytotoxicity plays an important role in immune response against infected cells, malignancy, and stressed cells, and involves in pathologic process in various diseases.

Numerous assays known in the art are used to determine the effect of the produced heterodimers on NK activation, including but not limited to:
Cytotoxicity assay—Killing of Target cells by NK cells (effector cells) in a co-culture assay. Percentage of killing is analyzed by flow cytometry analysis (FACS). Pre-labeled target cells (e.g. K562 PVR/PD-L1 cells or K562 WT cells) are placed in 96-wells plates and incubated at 37° C. with prelabeled primary NK cells at various effector-target (E:T) ratios. Optionally, NK cells are cultured with 1000 U/mL IL-2 for 48 hours before the assay. Cells are harvested following 4, 12 and/or 24 hours and assayed by flow cytometry. The numbers of target cells recovered from cultures without NK cells are used as a reference.
Cytotoxicity assay—Killing of Target cells by NK cells (effector cells) in a co-culture assay. Percentage of killing is determined by an Incucyte machine using labeled target cells and caspase sensitive florescent substrate.
Secretion of inflammatory cytokines: primary NK cells are stimulated with various target cells at various ratios for 24 hours. The levels of interferon γ (IFN-γ) and granulocyte-macrophage colony-stimulating factor (GM-CSF) in cell-free culture supernatants are determined with ELISA or Cytometric Bead Array (CBA).
Secretion of Granzyme B: primary NK cells are stimulated for 12 hours with various target cells at various ratios. The level of Granzyme B in cell-free culture supernatants is determined with ELISA.

### Results:

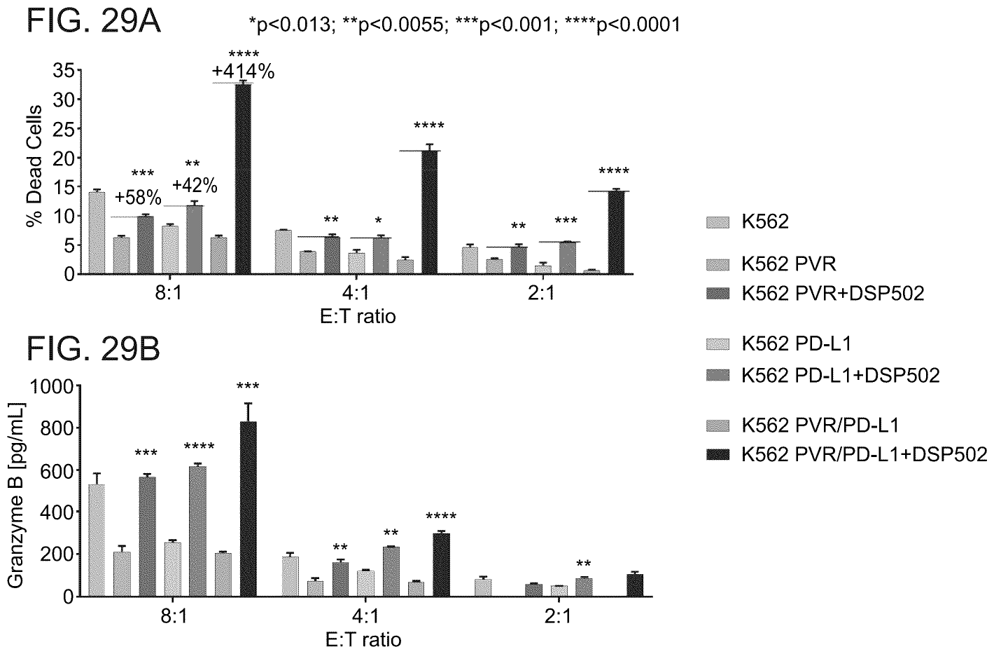

NK cells' killing of K562 WT target cells was higher compared to killing of K562 cells expressing PVR and/or PD-L1 (FIG. 29A). Addition of the TIGIT-PD1 heterodimer DSP502 to the co-culture comprising K562 WT cells as target cells did not increase NK cytotoxicity (data not shown). However, addition of DSP502 to the co-cultures comprising K562 cells expressing PVR and/or PD-L1 as target cells, significantly increased NK cytotoxicity, as compared to the non-treated cells, at all tested E:T ratios (FIG. 29A). The most significant cytotoxic effect was observed for the K562 cells expressing both PVR and PD-L1 treated with DSP502 heterodimer.

In line with the cytotoxicity assay described hereinabove, secretion of Granzyme B from NK cells co-cultured with K562 WT as target cells was higher compared to same with K562 cells expressing PVR and/or PD-L1 as target cells (FIG. 29B). Addition of DSP502 to the co-cultures comprising K562 cells expressing PVR and/or PD-L1 as target cells significantly increased Granzyme B secretion, as compared to the non-treated cells, at all tested E:T ratios (FIG. 29B). Same as with the cytotoxicity assay, the most significant increase in Granzyme B secretion was observed for the K562 cells expressing both PVR and PD-L1.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A heterodimer comprising two polypeptides selected from the group consisting of SIRPα, PD1, TIGIT, LILRB2 and SIGLEC10, wherein each of said two polypeptides is capable of binding a natural binding pair thereof, and wherein said heterodimer does not comprise an amino acid sequence of a type II membrane protein capable of binding a natural binding pair thereof.

2. The heterodimer of claim 1, wherein said heterodimer comprises a dimerizing moiety attached to said two polypeptides.

3. The heterodimer of claim 2, wherein said dimerizing moiety is an Fc domain of an antibody or a fragment thereof.

4. The heterodimer of claim 3, wherein said Fc domain is modified to alter it's binding to an Fc receptor, reduce an immune activating function thereof and/or improve half-life of said fusion.

5. (canceled)

6. The heterodimer of claim 1, wherein said heterodimer comprises said SIRPα polypeptide and said LILRB2 polypeptide.

7-8. (canceled)

9. The heterodimer of claim 1, wherein said heterodimer comprises said TIGIT polypeptide and said PD1 polypeptide.

10-14. (canceled)

15. The heterodimer of claim 1, wherein each of said polypeptides is a monomer in said heterodimer.

16. The heterodimer of claim 1, wherein said two polypeptides are comprised in a monomer of said heterodimer.

17. A composition comprising the heterodimer of claim 1, wherein said heterodimer is the predominant form of said two polypeptides in said composition.

18. A nucleic acid construct or system comprising at least one polynucleotide encoding the heterodimer of claim 1, and a regulatory element for directing expression of said polynucleotide in a host cell.

19. A host cell comprising the heterodimer of claim 1.

20. A method of producing a heterodimer, the method comprising introducing the nucleic acid construct or system of claim 18 to a host cell.

21. The method of claim 20, comprising isolating the heterodimer.

22. A method of treating a disease that can benefit from treatment with said heterodimer in a subject in need thereof, the method comprising administering to the subject a therapeutically effective amount of the heterodimer of claim 1, thereby treating the disease in the subject.

23. (canceled)

24. The method of claim 22, wherein said disease can benefit from activating immune cells.

25. The method of claim 22, wherein cells associated with said disease express said natural binding pair.

26. The method of claim 22, wherein said disease is cancer.

27. The method of claim 26, wherein said cancer is selected from the group consisting of lymphoma, leukemia, colon carcinoma, ovarian carcinoma, lung carcinoma, head and neck carcinoma and hepatocellular carcinoma.

28. (canceled)

29. A method of activating immune cells, the method comprising in-vitro activating immune cells in the presence of the heterodimer of claim 1.

30. The method of claim 29, wherein said activating is in the presence of cells expressing said natural binding pair.


SEQUENCE LISTING (164 sequences)

SEQ ID NO: 1 - protein, 585 aa, Artificial sequence
  SIRP Fc(IgG1 knob) 585AA first monomer of DSP120, DSP216, DSP403, DSP503
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - DNA, 1755 bp, Artificial sequence
  NA of #1
gaggaggagctgcaggtcatccagcccgataagtctgtgctggtggcagcaggagagaccgccacactgaggtgcaccgccacaagcctgatcccagtgggaccaatccagtggtttaggggagcaggccctggcagagagctgatctacaaccagaaggagggccacttcccaagagtgaccacagtgagcgacctgaccaagcggaacaatatggatttttccatcagaatcggcaatatcacacctgccgacgccggcacctactattgcgtgaagttcaggaagggctccccagacgatgtggagtttaagagcggagcaggcaccgagctgtccgtgcgggcaaagccttccgccccagtggtgtctggaccagcagccagagccaccccacagcacacagtgtccttcacctgtgagtctcacggctttagcccccgggacatcaccctgaagtggttcaagaacggcaatgagctgtctgactttcagaccaacgtggaccccgtgggcgagtctgtgagctattccatccactctacagccaaggtggtgctgacccgcgaggacgtgcacagccaggtcatctgcgaggtggcacacgtgaccctgcagggcgatcctctgaggggcacagccaatctgagcgagaccatcagagtgccccctacactggaggtgacccagcagcccgtgcgcgcagagaaccaagtgaatgtgacatgtcaggtgaggaagttctaccctcagcgcctgcagctgacctggctggagaacggcaacgtgagccggaccgagacagccagcaccgtgacagagaacaaggacggcacatataattggatgtcttggctgctggtgaacgtgagcgcccacagggacgatgtgaagctgacctgccaggtggagcacgacggacagccagccgtgtctaagagccacgatctgaaggtgagcgcccaccctaaggagcagggctccaacacagccgccgagaataccggcagcaacgagcggaatatctacggaggaggaggcagcggaggaggaggctccgagcctaagagctccgacaagacccacacatgcccaccatgtcctgcaccagagctgctgggaggaccttccgtgttcctgtttcctccaaagccaaaggatacactgatgatctccagaacaccagaggtgacctgcgtggtggtggacgtgtctcacgaggaccccgaggtgaagtttaactggtacgtggacggcgtggaggtgcacaatgccaagaccaagccaagggaggagcagtacaactccacatatcgcgtggtgtctgtgctgaccgtgctgcaccaggattggctgaacggcaaggagtataagtgtaaggtgagcaataaggccctgcccgcccctatcgagaagaccatctccaaggcaaagggacagcccagggagcctcaggtgtacacactgcccccttgccgcgacgagctgaccaagaaccaggtgtctctgtggtgtctggtgaagggcttctacccatctgacatcgccgtggagtgggagagcaatggccagcccgagaacaattacaagaccacaccacccgtgctggacagcgatggctccttctttctgtattccaagctgacagtggacaagtctcggtggcagcagggcaacgtgttttcctgttctgtgatgcacgaggccctgcacaatcactatacccagaagagcctgtccctgtctcccggcaag

SEQ ID NO: 3 - protein, 382 aa, Artificial sequence
  PD1 Fc (IgG1 hole) 382 AA second monomer of DSP120, DSP220, DSP402, DSP502
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 4 - DNA, 1146 bp, Artificial sequence
  NA of# 3
gattcacccgatagaccttggaacccacctaccttctcccccgccctgctggtggtgacagagggcgacaatgccaccttcacatgctcttttagcaacacctccgagtctttcgtgctgaattggtacaggatgagcccctccaaccagacagataagctggccgcatttccagaggaccgcagccagccaggacaggattcccggttcagagtgacccagctgcctaatggccgggactttcacatgtctgtggtgagagcccggagaaacgatagcggcacatacctgtgcggagccatctccctggcccctaaggcacagatcaaggagtccctgagggcagagctgagggtgaccgagaggagggcagaggtgccaacagcacacccttctccaagcccccggcctgcaggacagggaggaggaggctccggcggcggcggctctgagccaaagagctccgacaagacccacacatgcccaccatgtccagcaccagagctgctgggaggacctagcgtgttcctgtttcctccaaagccaaaggataccctgatgatctctaggaccccagaggtgacatgcgtggtggtggacgtgagccacgaggaccccgaggtgaagtttaattggtacgtggacggcgtggaggtgcacaacgccaagacaaagcctagggaggagcagtacaattctacctatcgcgtggtgagcgtgctgacagtgctgcaccaggattggctgaatggcaaggagtataagtgtaaggtgtccaacaaggccctgcctgccccaatcgagaagaccatctctaaggcaaagggacagccccgggagcctcaggtgtgcaccctgccccctagcagagacgagctgacaaagaatcaggtgtccctgtcttgtgccgtgaagggcttctaccccagcgacatcgcagtggagtgggagtccaacggacagcctgagaacaattataagaccacaccacccgtgctggactctgatggcagcttctttctggtgtccaagctgaccgtggacaagtctcggtggcagcagggcaacgtgtttagctgctccgtgatgcacgaagcactgcacaaccactacacccagaagtcactgtcactgtccccaggaaag

SEQ ID NO: 5 - protein, 582 aa, Artificial sequence
  SIRP alpha (IgG4 knob) 582 AA first monomer of DSP120V1, DSP216V1, DSP403V1, DSP503V1
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - DNA, 1746 bp, Artificial sequence
  NA of#5
gaggaggagctgcaggtcatccagcccgataagtctgtgctggtggcagcaggagagaccgccacactgagatgcaccgccacaagcctgatcccagtgggaccaatccagtggtttaggggagcaggacctggaagggagctgatctacaaccagaaggagggccacttcccaagggtgaccacagtgtccgacctgaccaagcggaacaatatggatttttctatcagaatcggcaatatcacacctgccgacgccggcacctactattgcgtgaagttcagaaagggcagcccagacgatgtggagtttaagtccggagcaggaaccgagctgtctgtgagagcaaagcctagcgccccagtggtgtccggaccagcagcaagggcaaccccacagcacacagtgtccttcacctgtgagtcccacggcttttctccacgcgatatcacactgaagtggttcaagaacggcaatgagctgagcgactttcagaccaacgtggatcccgtgggcgagtctgtgagctactccatccactctacagccaaggtggtgctgacccgggaggacgtgcacagccaggtcatctgcgaggtggcacacgtgaccctgcagggcgatcctctgagaggcacagccaatctgtccgagaccatcagggtgccccctacactggaggtgacccagcagcccgtgagggcagagaaccaagtgaatgtgacatgtcaggtgcggaagttctaccctcagagactgcagctgacctggctggagaacggcaatgtgagccgcaccgagacagcctccaccgtgacagagaacaaggacggcacatataattggatgagctggctgctggtgaacgtgtccgcccacagggacgatgtgaagctgacctgccaggtggagcacgacggacagccagccgtgtctaagagccacgatctgaaggtgtccgcccaccctaaggagcagggctctaacacagccgccgagaataccggcagcaacgagagaaatatctacggaggaggaggatccggaggaggaggatccgagtctaagtatggaccaccatgccctccatgtccagcacctgagtttgagggaggacctagcgtgttcctgtttccccctaagccaaaggacacactgatgatctccaggacaccagaggtgacctgcgtggtggtggacgtgtctcaggaggatcccgaggtgcagttcaactggtacgtggatggcgtggaggtgcacaatgccaagaccaagcctagggaggagcagtttaactctacataccgcgtggtgagcgtgctgaccgtgctgcaccaggattggctgaacggcaaggagtataagtgtaaggtgagcaataagggcctgccaagctccatcgagaagaccatctccaaggcaaagggacagccaagggagcctcaggtgtgcacactgccaccctctcaggaggagatgaccaagaaccaggtgagcctgtggtgtctggtgaagggcttctacccaagcgacatcgccgtggagtgggagtccaatggccagcccgagaacaattacaagaccacacctccagtgctggactctgatggcagcttctttctgtattctaggctgacagtggataagagccgctggcaggagggcaacgtgtttagctgttccgtgatgcacgaggccctgcacaatcactatacccagaagtctctgagcctgtccctgggcaag

SEQ ID NO: 7 - protein, 379 aa, Artificial sequence
  PD1 (IgG4 hole) 379 AA Second monomer of DSP120V1, DSP402V1, DSP502V2, DSP502V3
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 8 - DNA, 1137 bp, Artificial sequence
  NA of#7
gactctccagataggccttggaatccccctacctttagccccgccctgctggtggtgacagagggcgataacgccaccttcacatgctcttttagcaacacctccgagtctttcgtgctgaattggtacaggatgagcccttccaaccagacagacaagctggcagcatttcctgaggaccgcagccagccaggacaggattcccggttcagagtgacccagctgccaaatggcagggactttcacatgtctgtggtgcgcgcccggagaaacgatagcggcacatacctgtgcggagcaatctccctggcaccaaaggcacagatcaaggagtccctgagggcagagctgagggtgaccgagaggagggccgaggtgccaacagcacacccatctcctagcccaaggccagcaggacagggaggaggaggctctggaggaggaggatccgagtctaagtacggaccaccatgccctccatgtcctgcaccagagttcgagggaggaccatccgtgttcctgtttccacctaagcctaaggacaccctgatgatctccagaacccccgaggtgacatgcgtggtggtggacgtgtctcaggaggatcctgaggtgcagttcaattggtacgtggatggcgtggaggtgcacaacgccaagacaaagccccgggaggagcagtttaattctacctacagagtggtgagcgtgctgacagtgctgcaccaggattggctgaatggcaaggagtataagtgtaaggtgagcaacaagggcctgcctagctccatcgagaagaccatctccaaggccaagggccagccaagagagccccaggtgtacaccctgccacccagccagtgcgagatgacaaagaatcaggtgagcctgtcctgtgccgtgaagggcttctaccctagcgacatcgcagtggagtgggagtccaacggacagccagagaacaattataagaccacacctccagtgctggactccgatggctctttctttctggtgtcccggctgaccgtggataagagccggtggcaggagggcaacgtgttcagctgcagcgtgatgcacgaggccctgcacaaccactatacacagaagtccctgtctctgagcctgggcaag

SEQ ID NO: 9 - protein, 354 aa, Artificial sequence
  TIGIT Fc (IgG1 Knob) 354 AA first monomer of DSP205, DSP404, DSP502
MTGTIETTGNISAEKGGSIALQCHLSSTTAQVTQVNWEQQDQLLAISNADLGWHISPSFKDRVAPGPGLGLTLQSLTVNDTGEYFCIYHTYPDGTYTGRIFLEVLESSVAEHGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 1062 bp, Artificial sequence
  NA of#9
atgaccggcacaatcgagacaacaggcaacatctctgccgagaagggaggcagcatcgccctgcagtgccacctgagcagcaccacagcccaggtgacccaggtgaactgggagcagcaggaccagctgctggccatctctaatgccgatctgggctggcacatcagcccatcctttaaggatagggtggcaccaggaccaggcctgggcctgaccctgcagagcctgaccgtgaatgacacaggcgagtacttctgtatctaccacacatatcccgatggcacctatacaggcagaatctttctggaggtgctggagtctagcgtggccgagcacggaggaggaggcagcggaggaggaggctccgagcctaagtcctctgacaagacccacacatgccccccttgtcctgcaccagagctgctgggcggaccttccgtgttcctgtttccacccaagccaaaggataccctgatgatctccaggacccctgaggtgacatgcgtggtggtggacgtgtctcacgaggaccccgaggtgaagttcaactggtacgtggacggcgtggaggtgcacaatgccaagacaaagcctcgggaggagcagtacaactccacctatagagtggtgtctgtgctgacagtgctgcaccaggattggctgaacggcaaggagtataagtgtaaggtgagcaataaggccctgcccgcccctatcgagaaaaccatcagcaaggcaaagggacagccaagggagccacaggtgtacaccctgcctccatgccgcgacgagctgacaaagaaccaggtgagcctgtggtgtctggtgaagggcttctatccatctgacatcgccgtggagtgggagagcaatggccagcccgagaacaattacaagaccacaccccctgtgctggactccgatggctctttctttctgtatagcaagctgaccgtggacaagtccagatggcagcagggcaacgtgttttcttgcagcgtgatgcacgaggccctgcacaatcactacacacagaagtccctgtctctgagccccggcaag

SEQ ID NO: 11 - protein, 645 aa, Artificial sequence
  LILRB Fc (IgG1 hole) 645 AA second  monomer of DSP205, DSP216, DSP412
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 12 - DNA, 1935 bp, Artificial Sequence
  NA of #11
cagaccggcacaatcccaaagcccaccctgtgggccgagcctgattccgtgatcacccagggctctccagtgacactgtcctgccagggctctctggaggcccaggagtaccggctgtatagagagaagaagtctgccagctggatcacccggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagcggaggaggaggctccggaggaggaggctctggcggcggcggcagcgagcctaagagctccgacaagacccacacatgcccaccttgtccagcacctgagctgctgggaggaccatccgtgttcctgtttccacccaagcctaaggataccctgatgatctctcgcacccctgaggtgacatgcgtggtggtggacgtgagccacgaggaccccgaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtacaagtgtaaggtgtccaataaggccctgccagcccccatcgagaagaccatctctaaggcaaagggacagcccagggagcctcaggtgtgcaccctgcctccaagccgcgacgagctgacaaagaaccaggtgtctctgagctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctaggtggcagcagggcaacgtgttttcctgttctgtgatgcacgaggccctgcacaatcactacacacagaagagcctgtccctgtctcccggcaag

SEQ ID NO: 13 - protein, 351 aa, Artificial sequence
  TIGIT Fc (IgG4 knob) 351 AA first monomer of DSP205V1
MTGTIETTGNISAEKGGSIALQCHLSSTTAQVTQVNWEQQDQLLAISNADLGWHISPSFKDRVAPGPGLGLTLQSLTVNDTGEYFCIYHTYPDGTYTGRIFLEVLESSVAEHGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - DNA, 1053 bp, Artificial sequence
  NA of#13
atgaccggcacaatcgagacaacaggcaacatctctgccgagaagggaggcagcatcgccctgcagtgccacctgagcagcaccacagcccaggtgacccaggtgaactgggagcagcaggaccagctgctggccatctccaatgccgatctgggctggcacatcagcccctcctttaaggatagggtggcacctggaccaggcctgggcctgaccctgcagagcctgaccgtgaatgacacaggcgagtacttctgtatctaccacacatatcctgatggcacctatacaggcagaatctttctggaggtgctggagtctagcgtggccgagcacggaggaggaggctccggaggaggaggctctgagagcaagtacggaccaccttgcccaccatgtccagcacctgagttcgagggaggacctagcgtgttcctgtttcctccaaagccaaaggacaccctgatgatcagcaggacccctgaggtgacatgcgtggtggtggacgtgtcccaggaggaccccgaggtgcagttcaactggtatgtggatggcgtggaggtgcacaatgccaagacaaagcccagggaggagcagtttaactccacctaccgcgtggtgtctgtgctgacagtgctgcaccaggactggctgaacggcaaggagtataagtgtaaggtgtctaataagggcctgccctcctctatcgagaaaaccatcagcaaggccaagggccagccaagagagccacaggtgtgcaccctgccaccttcccaggaggagatgacaaagaaccaggtgtctctgtggtgtctggtgaagggcttctacccatctgacatcgccgtggagtgggagagcaatggccagcccgagaacaattacaagaccacaccacccgtgctggacagcgatggctccttctttctgtatagccggctgaccgtggataagtccagatggcaggagggcaacgtgttttcctgctctgtgatgcacgaggccctgcacaatcactatacacagaagagcctgtccctgtctctgggcaag

SEQ ID NO: 15 - protein, 642 aa, Artificial sequence
  LILRB Fc (IgG4 hole) 642 AA second monomer of DSP205V1, DSP216V1, DSP412V1
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 16 - DNA, 1926 bp, Artificial sequence
  NA of#15
cagaccggcacaatccctaagccaaccctgtgggccgagcctgatagcgtgatcacccagggctccccagtgacactgagctgccagggctccctggaggcacaggagtaccggctgtatagagagaagaagtctgccagctggatcacccggatcagacctgagctggtgaagaacggccagttccacatcccatctatcacctgggagcacacaggccggtacggatgccagtactatagccgggccagatggtctgagctgagcgaccccctggtgctggtcatgaccggagcctatcccaagcctacactgtctgcccagccaagcccagtggtgacctctggcggcagagtgacactgcagtgtgagagccaggtggccttcggcggctttatcctgtgcaaggagggcgaggaggagcacccacagtgtctgaatagccagccacacgccaggggcagctcccgcgccatcttcagcgtgggacccgtgagcccaaaccggagatggtcccaccgctgctacggctatgacctgaacagcccttacgtgtggtctagcccaagcgatctgctggagctgctggtgcccggcgtgagcaagaagccttccctgtctgtgcagcctggaccagtggtggcacctggagagtccctgaccctgcagtgcgtgagcgacgtgggctacgatcggtttgtgctgtataaggagggagagagggatctgaggcagctgccaggcagacagccacaggccggcctgtcccaggccaacttcaccctgggcccagtgagccggtcctatggcggccagtacagatgctatggcgcccacaatctgtcctctgagtgttccgccccaagcgaccccctggacatcctgatcaccggccagatcaggggcacaccctttatcagcgtgcagccaggacctaccgtggcctccggcgagaacgtgacactgctgtgccagagctggcgccagttccacacctttctgctgacaaaggcaggagcagcagacgcaccactgaggctgcgctccatccacgagtaccccaagtatcaggccgagttcccaatgtccccagtgacctctgcccacgcaggcacatacaggtgttatggcagcctgaacagcgacccctacctgctgtctcaccctagcgagccactggagctggtggtgtctggaggaggaggcagcggcggaggaggctccggaggcggcggctctgagagcaagtatggaccaccttgcccaccatgtccagcaccagagttcgagggaggaccaagcgtgttcctgtttcctccaaagcctaaggacaccctgatgatctcccgcacccctgaggtgacatgcgtggtggtggacgtgtctcaggaggaccccgaggtgcagtttaactggtacgtggatggcgtggaggtgcacaatgccaagaccaagccccgggaggagcagttcaactctacctacagagtggtgagcgtgctgacagtgctgcaccaggactggctgaacggcaaggagtataagtgtaaggtgagcaataagggcctgcctagctccatcgagaaaaccatcagcaaggcaaagggacagcccagggagcctcaggtgtataccctgcccccttcccagtgcgagatgacaaagaaccaggtgtccctgtcttgtgccgtgaagggcttttacccatccgacatcgccgtggagtgggagtctaatggccagcccgagaacaattataagaccacaccacccgtgctggactccgatggctctttctttctggtgagcaggctgaccgtggataagtcccgctggcaggagggcaacgtgttcagctgctccgtgatgcacgaggccctgcacaatcactacacacagaagtctctgagcctgtccctgggcaag

SEQ ID NO: 17 - protein, 585 aa, Artificial sequence
  SIRP alpha Fc (IgG1 knob as IgG4) 585AA first monomer of DSP216V2
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - DNA, 1755 bp, Artificial sequence
  NA of#17
gaggaggagctgcaggtcatccagcccgataagtctgtgctggtggcagcaggagagaccgccacactgaggtgcaccgccacaagcctgatcccagtgggaccaatccagtggtttaggggagcaggccctggcagagagctgatctacaaccagaaggagggccacttcccaagagtgaccacagtgagcgacctgaccaagcggaacaatatggatttttccatcagaatcggcaatatcacacctgccgacgccggcacctactattgcgtgaagttcaggaagggctccccagacgatgtggagtttaagagcggagcaggcaccgagctgtccgtgcgggcaaagccttccgccccagtggtgtctggaccagcagccagagccaccccacagcacacagtgtccttcacctgtgagtctcacggctttagcccccgggacatcaccctgaagtggttcaagaacggcaatgagctgtctgactttcagaccaacgtggaccccgtgggcgagtctgtgagctattccatccactctacagccaaggtggtgctgacccgcgaggacgtgcacagccaggtcatctgcgaggtggcacacgtgaccctgcagggcgatcctctgaggggcacagccaatctgagcgagaccatcagagtgccccctacactggaggtgacccagcagcccgtgcgcgcagagaaccaagtgaatgtgacatgtcaggtgaggaagttctaccctcagcgcctgcagctgacctggctggagaacggcaacgtgagccggaccgagacagccagcaccgtgacagagaacaaggacggcacatataattggatgtcttggctgctggtgaacgtgagcgcccacagggacgatgtgaagctgacctgccaggtggagcacgacggacagccagccgtgtctaagagccacgatctgaaggtgagcgcccaccctaaggagcagggctccaacacagccgccgagaataccggcagcaacgagcggaatatctacggaggaggaggcagcggaggaggaggctccgagcctaagagctccgacaagacccacacatgcccaccatgtcctgcaccagagctgctgggaggaccttccgtgttcctgtttcctccaaagccaaaggatacactgatgatctccagaacaccagaggtgacctgcgtggtggtggacgtgtctcacgaggaccccgaggtgaagtttaactggtacgtggacggcgtggaggtgcacaatgccaagaccaagccaagggaggagcagtacaactccacatatcgcgtggtgtctgtgctgaccgtgctgcaccaggattggctgaacggcaaggagtataagtgtaaggtgagcaataaggccctgcccgcccctatcgagaagaccatctccaaggcaaagggacagcccagggagcctcaggtgtgcacactgcccccttcccgcgacgagctgaccaagaaccaggtgtctctgtggtgtctggtgaagggcttctacccatctgacatcgccgtggagtgggagagcaatggccagcccgagaacaattacaagaccacaccacccgtgctggacagcgatggctccttctttctgtattccaagctgacagtggacaagtctcggtggcagcagggcaacgtgttttcctgttctgtgatgcacgaggccctgcacaatcactatacccagaagagcctgtccctgtctcccggcaag

SEQ ID NO: 19 - protein, 645 aa, Artificial sequence
  LILRB2 FC (IgG1 hole as IgG4) 645 AA second monomer of DSP216V2
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 20 - DNA, 1935 bp, Artificial sequence
  NA of#19
cagaccggcacaatcccaaagcccaccctgtgggccgagcctgattccgtgatcacccagggctctccagtgacactgtcctgccagggctctctggaggcccaggagtaccggctgtatagagagaagaagtctgccagctggatcacccggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagcggaggaggaggctccggaggaggaggctctggcggcggcggcagcgagcctaagagctccgacaagacccacacatgcccaccttgtccagcacctgagctgctgggaggaccatccgtgttcctgtttccacccaagcctaaggataccctgatgatctctcgcacccctgaggtgacatgcgtggtggtggacgtgagccacgaggaccccgaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtacaagtgtaaggtgtccaataaggccctgccagcccccatcgagaagaccatctctaaggcaaagggacagcccagggagcctcaggtgtataccctgcctccaagccgctgcgagctgacaaagaaccaggtgtctctgagctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctaggtggcagcagggcaacgtgttttcctgttctgtgatgcacgaggccctgcacaatcactacacacagaagagcctgtccctgtctcccggcaag

SEQ ID NO: 21 - protein, 645 aa, Artificial sequence
  LILRB2 Fc (IgG1 knob) 645 AA first monomer of DSP220
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 642 aa, Artificial sequence
  LILRB2 Fc IgG4 knob 642 AA first monomer of DSP220V1
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - DNA, 1926 bp, Artificial sequence
  NA of#22
cagaccggcacaatccccaagcctaccctgtgggccgagccagatagcgtgatcacccagggctcccccgtgacactgtcttgccagggcagcctggaggcacaggagtaccggctgtatagagagaagaagagcgcctcctggatcacccggatcagacccgagctggtgaagaacggccagtttcacatcccttccatcacctgggagcacacaggccggtacggatgccagtactattctcgggccagatggagcgagctgtccgaccccctggtgctggtcatgaccggcgcctatccaaagcccacactgtccgcccagccttctccagtggtgacctctggcggcagagtgacactgcagtgtgagagccaggtggccttcggcggctttatcctgtgcaaggagggcgaggaggagcacccccagtgtctgaatagccagcctcacgcccggggcagctccagagccatcttctctgtgggccctgtgagcccaaaccggagatggtcccacaggtgctacggctatgacctgaacagcccatacgtgtggtctagcccctctgatctgctggagctgctggtgcctggcgtgagcaagaagccatctctgagcgtgcagccaggccctgtggtggcacctggcgagtctctgaccctgcagtgcgtgagcgacgtgggctacgatcggttcgtgctgtataaggagggagagagggatctgaggcagctgccaggcagacagcctcaggcaggactgtcccaggcaaactttacactgggccccgtgagccggagctacggcggacagtaccgctgctatggagcacacaatctgtcctctgagtgttctgcccccagcgaccccctggacatcctgatcaccggccagatcaggggcacaccattcatcagcgtgcagccaggaccaaccgtggcctccggcgagaacgtgacactgctgtgccagagctggcgccagttccacacctttctgctgacaaaggcaggagcagcagacgcacctctgaggctgcgctccatccacgagtacccaaagtatcaggccgagtttccaatgagccccgtgacctccgcccacgcaggcacatacagatgctatggcagcctgaacagcgacccctacctgctgagccacccttccgagccactggagctggtggtgtccggcggcggcggctctggcggaggaggcagcggaggaggaggatccgagtctaagtacggaccaccatgccctccatgtcctgcaccagagttcgagggaggaccatccgtgttcctgtttccacctaagcctaaggacaccctgatgatctccagaacccccgaggtgacatgcgtggtggtggacgtgtctcaggaggatcctgaggtgcagttcaattggtacgtggatggcgtggaggtgcacaacgccaagacaaagccccgggaggagcagtttaatagcacctacagagtggtgtccgtgctgacagtgctgcaccaggattggctgaatggcaaggagtataagtgtaaggtgagcaacaagggcctgcctagctccatcgagaagaccatctccaaggccaagggccagccaagagagccacaggtgtgcaccctgccaccaagccaggaggagatgacaaagaatcaggtgtccctgtggtgtctggtgaagggcttctacccttccgacatcgccgtggagtgggagtctaacggccagccagagaacaattacaagaccacacctccagtgctggactctgatggcagcttctttctgtattctcggctgaccgtggataagagcagatggcaggagggcaacgtgttcagctgctccgtgatgcacgaggccctgcacaaccactatacacagaagtctctgagcctgtccctgggcaag

SEQ ID NO: 24 - protein, 375 aa, Artificial sequence
  SIGLEC10 Fc (IgG1 knob) 375 AA first monomer of DSP402, DSP412
DGRFWIRVQESVMVPEGLSISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 367 aa, Artificial sequence
  SIGLEC10 Fc (IgG4) 367 AA first monomer of DSP402V1, DSP412V1
DGRFWIRVQESVMVPEGLSISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - DNA, 1101 bp, Artificial sequence
  NA of#25
gatggccggttttggatcagagtgcaggagtccgtgatggtgcctgagggcctgtctatcagcgtgccatgctccttctcttaccccagacaggactggaccggctctacacccgcctacggctattggtttaaggccgtgaccgagacaacaaagggcgcccctgtggccacaaaccaccagagcagagaggtggagatgtccacccggggcagattccagctgacaggcgaccccgccaagggcaattgtagcctggtcatcagggacgcccagatgcaggatgagtctcagtacttctttagggtggagcgcggcagctacgtgcgctataactttatgaatgatggcttctttctgaaggtgaccgccctgacacagaagggaggaggaggctccggcggaggaggcagcgagtccaagtatggaccaccttgcccaccatgtcctgcaccagagttcgagggaggacctagcgtgttcctgtttcctccaaagccaaaggacaccctgatgatcagcaggactcctgaggtgacatgcgtggtggtggacgtgtcccaggaggaccccgaggtgcagttcaactggtatgtggatggcgtggaggtgcacaatgccaagacaaagccacgggaggagcagtttaactctacctacagagtggtgagcgtgctgacagtgctgcaccaggattggctgaacggcaaggagtataagtgtaaggtgtctaataagggcctgcccagctccatcgagaaaaccatcagcaaggcaaagggacagccccgggagcctcaggtgtgcaccctgcccccttcccaggaggagatgacaaagaaccaggtgtctctgtggtgtctggtgaagggcttctacccaagcgacatcgccgtggagtgggagtccaatggccagcccgagaacaattacaagaccacaccacccgtgctggactccgatggctctttctttctgtattccaggctgaccgtggataagtctcgctggcaggagggcaacgtgttttcttgcagcgtgatgcacgaggccctgcacaatcactatacacagaagtccctgtctctgagcctgggcaag

SEQ ID NO: 27 - protein, 232 aa, Artificial sequence
  hIgG1 Fc linker (knob V1 only in DSP216V2) 232 aa
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 232 aa, Artificial sequence
  hIgG1 Fc linker (hole V1 only in DSP216V2) 232 aa
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 375 aa, Artificial sequence
  SIGLEC10 Fc (IgG1 hole) 475 AA first monomer of DSP404, DSP403
DGRFWIRVQESVMVPEGLSISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 30 - protein, 369 aa, Artificial sequence
  SIGLEC10 Fc (IgG4 hole) second monomer of DSP404V1, DSP403V1
MTGTIETTGNISAEKGGSIALQCHLSSTTAQVTQVNWEQQDQLLAISNADLGWHISPSFKDRVAPGPGLGLTLQSLTVNDTGEYFCIYHTYPDGTYTGRIFLEVLESSVAEHGGGGSGGGGSEPKSSDKTHTCPPCPAPEESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 31 - protein, 351 aa, Artificial sequence
  TIGIT No I42D Fc (IgG4 knob) 351 AA first monomer of DSP502V2
MTGTIETTGNISAEKGGSIILQCHLSSTTAQVTQVNWEQQDQLLAISNADLGWHISPSFKDRVAPGPGLGLTLQSLTVNDTGEYFCIYHTYPDGTYTGRIFLEVLESSVAEHGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - DNA, 1053 bp, Artificial sequence
  NA of#31
atgaccggcacaatcgagacaacaggcaacatctctgccgagaagggaggcagcatcatcctgcagtgccacctgagcagcaccacagcccaggtgacccaggtgaactgggagcagcaggaccagctgctggccatctccaatgccgatctgggctggcacatcagcccctcctttaaggatagggtggcacctggaccaggcctgggcctgaccctgcagagcctgaccgtgaatgacacaggcgagtacttctgtatctaccacacatatcctgatggcacctatacaggcagaatctttctggaggtgctggagtctagcgtggccgagcacggaggaggaggctccggaggaggaggctctgagagcaagtacggaccaccttgcccaccatgtccagcacctgagttcgagggaggacctagcgtgttcctgtttcctccaaagccaaaggacaccctgatgatcagcaggacccctgaggtgacatgcgtggtggtggacgtgtcccaggaggaccccgaggtgcagttcaactggtatgtggatggcgtggaggtgcacaatgccaagacaaagcccagggaggagcagtttaactccacctaccgcgtggtgtctgtgctgacagtgctgcaccaggactggctgaacggcaaggagtataagtgtaaggtgtctaataagggcctgccctcctctatcgagaaaaccatcagcaaggccaagggccagccaagagagccacaggtgtgcaccctgccaccttcccaggaggagatgacaaagaaccaggtgtctctgtggtgtctggtgaagggcttctacccatctgacatcgccgtggagtgggagagcaatggccagcccgagaacaattacaagaccacaccacccgtgctggacagcgatggctccttctttctgtatagccggctgaccgtggataagtccagatggcaggagggcaacgtgttttcctgctctgtgatgcacgaggccctgcacaatcactatacacagaagagcctgtccctgtctctgggcaag

SEQ ID NO: 33 - protein, 355 aa, Artificial sequence
  TIGIT WT Fc (IgG4 knob) 355AA first monomer of DSP502V3
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - DNA, 1065 bp, Artificial Sequence
  NA of #33
atgatgaccggcactattgaaactaccggcaacatctctgccgagaagggcggcagcatcatcctccagtgccacctgagcagcaccacagcccaggtgacacaggtgaactgggagcagcaggaccagctgctggccatctgtaatgccgatctgggctggcacatcagcccttccttcaaggacagggtggcccctggcccaggcctgggcctgaccctccagagcctgaccgtgaatgacacaggcgagtacttctgcatctaccacacatatccagatggcacctatacaggccggatctttctggaggtgctggagtctagcgtggcagagcacggcgccagaggcggaggaggcagcggaggaggaggctctgagagcaagtacggccctccttgcccaccatgtccagcacctgagtttgagggcggcccttccgtgttcctgtttcctccaaagccaaaggacaccctgatgatcagcaggaccccagaggtgacatgcgtggtggtggacgtgtcccaggaggaccccgaggtgcagttcaactggtatgtggatggcgtggaggtgcacaatgccaagacaaagcccagggaggagcagtttaactccacctaccgcgtggtgtctgtgctgacagtgctgcaccaggattggctgaacggcaaggagtataagtgtaaggtgtctaataagggcctgccttcctctatcgagaaaaccatcagcaaggcaaagggacagccacgcgagccacaggtgtgcaccctgcccccttcccaggaggagatgacaaagaaccaggtgtctctgtggtgtctggtgaagggcttctacccctctgacatcgccgtggagtgggagagcaatggccagcctgagaacaattacaagaccacaccacccgtgctggacagcgatggctccttctttctgtatagccggctgaccgtggataagtccagatggcaggagggcaacgtgttcagctgctccgtgatgcacgaagcactgcacaatcattacactcagaagtccctgtccctgtcactgggcaag

SEQ ID NO: 35 - protein, 354 aa, Artificial sequence
  TIGIT Fc (IgG1 Hole) 354 AA second monomer of DSP503
MTGTIETTGNISAEKGGSIALQCHLSSTTAQVTQVNWEQQDQLLAISNADLGWHISPSFKDRVAPGPGLGLTLQSLTVNDTGEYFCIYHTYPDGTYTGRIFLEVLESSVAEHGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 351 aa, Artificial sequence
  TIGIT Fc (IgG4 Hole) 351 AA second monomer of DSP503V1
MTGTIETTGNISAEKGGSIALQCHLSSTTAQVTQVNWEQQDQLLAISNADLGWHISPSFKDRVAPGPGLGLTLQSLTVNDTGEYFCIYHTYPDGTYTGRIFLEVLESSVAEHGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 37 - protein, 288 aa, Artificial sequence
  aa sequence of full length PD1
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 38 - DNA, 867 bp, Artificial Sequence
  na sequence of full length PD1
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctga

SEQ ID NO: 39 - protein, 150 aa, Artificial sequence
  PD1 ECD Full with CYS93>Ser substitution
PGWFLDSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQFQTLV

SEQ ID NO: 40 - DNA, 450 bp, Artificial sequence
  na sequence encoding SEQ ID NO39
cccggctggtttctggactctccagacagaccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactctcggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcaccttctccaagacctgctggccagtttcagacactcgtg

SEQ ID NO: 41 - protein, 167 aa, Artificial sequence
  PD1 ECM KNOWN FRAGMENT
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQ

SEQ ID NO: 42 - protein, 122 aa, Artificial sequence
  PD1 ECM KNOWN FRAGMENT
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTER

SEQ ID NO: 43 - protein, 150 aa, Artificial sequence [sequence omitted]

SEQ ID NO: 44 - DNA, 450 bp, Artificial sequence
  na pf SEQ ID NO 43
ccaggatggttcttagactctccagataggccttggaatccccctacctttagccccgccctgctggtggtgacagagggcgataacgccaccttcacatgctcttttagcaacacctccgagtctttcgtgctgaattggtacaggatgagcccttccaaccagacagacaagctggcagcatttcctgaggaccgctcccagccaggccaggattgccggttcagagtgacccagctgccaaatggcagggactttcacatgagcgtggtgcgcgcccggagaaacgattccggcacatacctgtgcggagcaatctctctggcaccaaaggcacagatcaaggagtccctgagggcagagctgagggtgaccgagaggagggccgaggtgccaacagcacacccatctcctagcccaaggccagcaggacagttccaaaccctggtg

SEQ ID NO: 45 - protein, 150 aa, Artificial sequence; identical to SEQ ID NO: 39
  150 aa ORIGINAL PD1 DOMAIN with CYS93>Ser substitution

SEQ ID NO: 46 - DNA, 450 bp, Artificial sequence
  na of SEQ ID NO  45
ccaggatggttcttagactctccagataggccttggaatccccctacctttagccccgccctgctggtggtgacagagggcgataacgccaccttcacatgctcttttagcaacacctccgagtctttcgtgctgaattggtacaggatgagcccttccaaccagacagacaagctggcagcatttcctgaggaccgctcccagccaggccaggattctcggttcagagtgacccagctgccaaatggcagggactttcacatgagcgtggtgcgcgcccggagaaacgattccggcacatacctgtgcggagcaatctctctggcaccaaaggcacagatcaaggagtccctgagggcagagctgagggtgaccgagaggagggccgaggtgccaacagcacacccatctcctagcccaaggccagcaggacagttccaaaccctggtg

SEQ ID NO: 47 - protein, 140 aa, Artificial sequence
  140 aa -5-5 in PD1 DOMAIN
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQ

SEQ ID NO: 48 - DNA, 419 bp, Artificial sequence
  na of SEQ ID NO 47
gactctccagataggccttggaatccccctacctttagccccgccctgctggtggtgacagagggcgataacgccaccttcacatgctcttttagcaacacctccgagtctttcgtgctgaattggtacaggatgagcccttccaaccagacagacaagctggcagcatttcctgaggaccgctcccagccaggccaggattgccggttcagagtgacccagctgccaaatggcagggactttcacatgagcgtggtgcgcgcccggagaaacgattccggcacatacctgtgcggagcaatctctctggcaccaaaggcacagatcaaggagtccctgagggcagagctgagggtgaccgagaggagggccgaggtgccaacagcacacccatctcctagcccaaggccagcaggaca

SEQ ID NO: 49 - protein, 140 aa, Artificial sequence
  140 aa -5-5 in PD1 DOMAIN with CYS93>Ser substitution
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQ

SEQ ID NO: 50 - DNA, 419 bp, Artificial sequence
  na of SEQ ID NO 49
gactctccagataggccttggaatccccctacctttagccccgccctgctggtggtgacagagggcgataacgccaccttcacatgctcttttagcaacacctccgagtctttcgtgctgaattggtacaggatgagcccttccaaccagacagacaagctggcagcatttcctgaggaccgctcccagccaggccaggatctccggttcagagtgacccagctgccaaatggcagggactttcacatgagcgtggtgcgcgcccggagaaacgattccggcacatacctgtgcggagcaatctctctggcaccaaaggcacagatcaaggagtccctgagggcagagctgagggtgaccgagaggagggccgaggtgccaacagcacacccatctcctagcccaaggccagcaggaca

SEQ ID NO: 51 - protein, 232 aa, Artificial sequence
  hIgG1 Fc linker (knob) 232 aa
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 232 aa, Artificial sequence
  hIgG1 Fc linker (hole) 232 aa
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 53 - protein, 128 aa, Artificial sequence
  128 aa PD1 segment
PWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSP

SEQ ID NO: 54 - DNA, 384 bp, Artificial sequence
  NA OF #53
ccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactgccggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcacct

SEQ ID NO: 55 - protein, 135 aa, Artificial sequence
  135 aa PD1 segment
PWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQ

SEQ ID NO: 56 - DNA, 405 bp, Artificial sequence
  NA OF #55
ccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactgccggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcaccttctccaagacctgccggccag

SEQ ID NO: 57 - protein, 133 aa, Artificial sequence
  133 aa PD1 segment
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSP

SEQ ID NO: 58 - DNA, 399 bp, Artificial sequence
  NA OF #57
gactcccctgacagaccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactgccggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcacct

SEQ ID NO: 59 - protein, 135 aa, Artificial sequence
  PD1 -10-5 (with CYS93>Ser substitution)
PWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQ

SEQ ID NO: 60 - DNA, 405 bp, Artificial sequence
  NA OF #59
ccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactctcggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcaccttctccaagacctgctggccag

SEQ ID NO: 61 - protein, 128 aa, Artificial sequence
  PD1 -10-12 (with CYS93>Ser substitution)
PWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSP

SEQ ID NO: 62 - DNA, 384 bp, Artificial sequence
  NA OF #61
ccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactctcggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcacct

SEQ ID NO: 63 - protein, 133 aa, Artificial sequence
  PD1 -5-12 (New, from V20) (with CYS93>Ser substitution)
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSP

SEQ ID NO: 64 - DNA, 399 bp, Artificial sequence
  NA OF #63
gactctccagacagaccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactctcggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcacct

SEQ ID NO: 65 - protein, 127 aa, Artificial sequence
  PD1 -11-12
WNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSP

SEQ ID NO: 66 - DNA, 381 bp, Artificial sequence
  NA OF #65
tggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactgtcggttcagagtgacccagctgcctaacggcagagacttccacatgtccgtcgtgcgggccagaagaaacgactctggcacctatctgtgcggcgccatctctctggctcccaaggctcagatcaaagagtctctgcgggccgagctgagagtgacagaaagacgagctgaggtgcccaccgctcatccctcacct

SEQ ID NO: 67 - protein, 127 aa, Artificial sequence
  PD1 -11-12 (New, from V18) (with CYS93>Ser substitution)
WNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSP

SEQ ID NO: 68 - DNA, 381 bp, Artificial sequence
  NA OF #67
tggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactctcggttcagagtgacccagctgcctaacggcagagacttccacatgtccgtcgtgcgggccagaagaaacgactctggcacctatctgtgcggcgccatctctctggctcccaaggctcagatcaaagagtctctgcgggccgagctgagagtgacagaaagacgagctgaggtgcccaccgctcatccctcacct

SEQ ID NO: 69 - protein, 134 aa, Artificial sequence
  PD1 -11-5
WNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQ

SEQ ID NO: 70 - DNA, 402 bp, Artificial sequence
  NA OF #69
tggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactgtcggttcagagtgacccagctgcctaacggcagagacttccacatgtccgtcgtgcgggccagaagaaacgactctggcacctatctgtgcggcgccatctctctggctcccaaggctcagatcaaagagtctctgcgggccgagctgagagtgacagaaagacgagctgaggtgcccaccgctcatccctcaccttctccaagacctgctggccag

SEQ ID NO: 71 - protein, 134 aa, Artificial sequence
  PD1 -11-5 (New, from V19) (with CYS93>Ser substitution)
WNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQ

SEQ ID NO: 72 - DNA, 402 bp, Artificial sequence
  NA OF #71
tggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactctcggttcagagtgacccagctgcctaacggcagagacttccacatgtccgtcgtgcgggccagaagaaacgactctggcacctatctgtgcggcgccatctctctggctcccaaggctcagatcaaagagtctctgcgggccgagctgagagtgacagaaagacgagctgaggtgcccaccgctcatccctcaccttctccaagacctgctggccag

SEQ ID NO: 73 - protein, 138 aa, Artificial sequence
  PD1 -5-7
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPA

SEQ ID NO: 74 - DNA, 414 bp, Artificial sequence
  NA OF #73
gactctccagacagaccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactgtcggttcagagtgacccagctgcctaacggcagagacttccacatgtccgtcgtgcgggccagaagaaacgactctggcacctatctgtgcggcgccatctctctggctcccaaggctcagatcaaagagtctctgcgggccgagctgagagtgacagaaagacgagctgaggtgcccaccgctcatccctcaccttctccaagacctgct

SEQ ID NO: 75 - protein, 138 aa, Artificial sequence
  PD1 -5-7 (New, from V21) (with CYS93>Ser substitution)
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPA

SEQ ID NO: 76 - DNA, 414 bp, Artificial sequence
  NA OF #75
gactctccagacagaccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactctcggttcagagtgacccagctgcctaacggcagagacttccacatgtccgtcgtgcgggccagaagaaacgactctggcacctatctgtgcggcgccatctctctggctcccaaggctcagatcaaagagtctctgcgggccgagctgagagtgacagaaagacgagctgaggtgcccaccgctcatccctcaccttctccaagacctgct

SEQ ID NO: 77 - protein, 136 aa, Artificial sequence
  PD1 -5-9 from (with out CYS93>Ser substitution)
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPR

SEQ ID NO: 78 - DNA, 408 bp, Artificial sequence
  NA OF #77
gactctccagacagaccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactgtcggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcaccttctccaaga

SEQ ID NO: 79 - protein, 145 aa, Artificial sequence
  PD1 -0-5 from (with out CYS93>Ser substitution)
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQ

SEQ ID NO: 80 - DNA, 435 bp, Artificial sequence
  NA OF #79
cccggctggtttctggactctccagacagaccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactgtcggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcaccttctccaagacctgctggccag

SEQ ID NO: 81 - protein, 143 aa, Artificial sequence
  PD1 -0-7 from (with out CYS93>Ser substitution) 143 aa
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPA

SEQ ID NO: 82 - DNA, 429 bp, Artificial sequence
  NA OF #81
cccggctggtttctggactctccagacagaccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactgtcggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcaccttctccaagacctgct

SEQ ID NO: 83 - protein, 504 aa, Artificial sequence
  aa sequence of full length SIRP alpha
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 84 - DNA, 1512 bp, Artificial Sequence
  NA OF #83
atggagcccgccggcccggcccccggccgcctcgggccgctgctctgcctgctgctcgccgcgtcctgcgcctggtcaggagtggcgggtgaggaggagctgcaggtgattcagcctgacaagtccgtatcagttgcagctggagagtcggccattctgcactgcactgtgacctccctgatccctgtggggcccatccagtggttcagaggagctggaccagcccgggaattaatctacaatcaaaaagaaggccacttcccccgggtaacaactgtttcagagtccacaaagagagaaaacatggacttttccatcagcatcagtaacatcaccccagcagatgccggcacctactactgtgtgaagttccggaaagggagccctgacacggagtttaagtctggagcaggcactgagctgtctgtgcgtgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtga

SEQ ID NO: 85 - protein, 343 aa, Artificial sequence
  ORIGINAL SIRPa DOMAIN (343AA)
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 86 - DNA, 1029 bp, Artificial Sequence
  NA OF #85
gaggaggagctgcaggtgattcagcctgacaagtccgtatcagttgcagctggagagtcggccattctgcactgcactgtgacctccctgatccctgtggggcccatccagtggttcagaggagctggaccagcccgggaattaatctacaatcaaaaagaaggccacttcccccgggtaacaactgtttcagagtccacaaagagagaaaacatggacttttccatcagcatcagtaacatcaccccagcagatgccggcacctactactgtgtgaagttccggaaagggagccctgacacggagtttaagtctggagcaggcactgagctgtctgtgcgtgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatatt

SEQ ID NO: 87 - protein, 116 aa, Artificial sequence
  116 aa SIRPa segment
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRA

SEQ ID NO: 88 - DNA, 348 bp, Artificial sequence
  NA OF #87
gaagaggaactgcaagtgatccagcctgacaagtccgtgctggtggctgctggcgaaaccgccacactgagatgtaccgccacctctctgatccctgtgggccctatccagtggtttagaggcgctggacctggcagagagctgatctacaaccagaaagagggccactttcctagagtgaccaccgtgtccgacctgaccaagcggaacaacatggacttctccatccggatcggcaacatcacccctgctgatgccggcacctactactgcgtgaagttccggaagggctcccctgacgacgtcgagtttaaatccggcgctggcaccgaactgtccgtgcgagct

SEQ ID NO: 89 - protein, 343 aa, Artificial sequence
  343 amino acids sequence of SIRPa with 4 point mutations
EEEIQVIQPDKSVLVAAGETATLRCTITSLIPVGPIQWFRGAGPGRVLIYNQKEGHFPRVTTVSDLTKRNNMDFSIRIGNITPADAGTYYCIKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 90 - DNA, 1029 bp, Artificial sequence
  NA OF #89
gaagaggaaatccaagtgatccagcctgacaagtccgtgctggtggctgctggcgaaaccgccacactgagatgtaccatcacctctctgatccctgtgggccctatccagtggtttagaggcgctggacctggcagagtgctgatctacaaccagaaagagggccactttcctagagtgaccaccgtgtccgacctgaccaagcggaacaacatggacttctccatccggatcggcaacatcacccctgctgatgccggcacctactactgcatcaagttccggaagggctcccctgacgacgtcgagtttaaatccggcgctggcaccgaactgtccgtgcgagctaaaccttctgctcccgtggtgtctggccctgccgctagagctacacctcagcacaccgtgtcttttacctgcgagtcccacggcttcagccctagagacatcaccctgaagtggttcaagaacggcaacgagctgtccgacttccagaccaacgtggaccctgtgggagagtccgtgtcctactccatccactctaccgccaaggtggtgctgacccgagaggacgtgcacagccaagtgatctgtgaagtggcccacgtgaccctccagggcgatcctttgagaggcaccgccaacctgtccgagacaatcagagtgcctcctacactggaagtgacccagcagcctgtgcgggccgagaatcaagtgaacgtgacctgccaagtgcggaagttctaccctcagagactgcagctgacctggctggaaaacggcaatgtgtccagaaccgagacagcctccaccgtgaccgagaacaaggatggcacctacaattggatgtcctggctgctcgtgaacgtgtccgctcacagagatgacgtgaagctgacatgccaggtggaacacgatggccagcctgccgtgtctaagtcccacgacctgaaagtgtctgctcaccccaaagagcagggctccaataccgccgctgagaacaccggctccaacgagagaaacatctac

SEQ ID NO: 91 - protein, 116 aa, Artificial sequence
  116 amino acids sequence of SIRPa with 4 point mutations
EEEIQVIQPDKSVLVAAGETATLRCTITSLIPVGPIQWFRGAGPGRVLIYNQKEGHFPRVTTVSDLTKRNNMDFSIRIGNITPADAGTYYCIKFRKGSPDDVEFKSGAGTELSVRA

SEQ ID NO: 92 - DNA, 348 bp, Artificial sequence
  NA OF #91
gaagaggaaatccaagtgatccagcctgacaagtccgtgctggtggctgctggcgaaaccgccacactgagatgtaccatcacctctctgatccctgtgggccctatccagtggtttagaggcgctggacctggcagagtgctgatctacaaccagaaagagggccactttcctagagtgaccaccgtgtccgacctgaccaagcggaacaacatggacttctccatccggatcggcaacatcacccctgctgatgccggcacctactactgcatcaagttccggaagggctcccctgacgacgtcgagtttaaatccggcgctggcaccgaactgtccgtgcgagct

SEQ ID NO: 93 - protein, 117 aa, Artificial sequence
  aa SIRPa segment - addition K at the C-ter of SEQ ID 87  (117 aa SIRPa segment)
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAK

SEQ ID NO: 94 - DNA, 351 bp, Artificial sequence
  NA OF #93
gaagaggaactgcaagtgatccagcctgacaagtccgtgctggtggctgctggcgaaaccgccacactgagatgtaccgccacctctctgatccctgtgggccctatccagtggtttagaggcgctggacctggcagagagctgatctacaaccagaaagagggccactttcctagagtgaccaccgtgtccgacctgaccaagcggaacaacatggacttctccatccggatcggcaacatcacccctgctgatgccggcacctactactgcgtgaagttccggaagggctcccctgacgacgtcgagtttaaatccggcgctggcaccgaactgtccgtgcgagctaag

SEQ ID NO: 95 - protein, 440 aa, Artificial Sequence
  LILRB2 Full ECD AA SEQUENCE
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGV

SEQ ID NO: 96 - protein, 398 aa, Artificial sequence
  LILRB2 D1-4, AA 22-419 (Q8N423-1 UniParc) AA SEQUENCE
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVS

SEQ ID NO: 97 - DNA, 1194 bp, Artificial sequence
  LILRB2 D1-4, AA 22-419 (Q8N423-1 UniParc) NA SEQUENCE
cagaccggcacaatccccaagcctaccctgtgggccgagccagatagcgtgatcacccagggctcccccgtgacactgtcttgccagggcagcctggaggcacaggagtaccggctgtatagagagaagaagagcgcctcctggatcacccggatcagacccgagctggtgaagaacggccagtttcacatcccttccatcacctgggagcacacaggccggtacggatgccagtactattctcgggccagatggagcgagctgtccgaccccctggtgctggtcatgaccggcgcctatccaaagcccacactgtccgcccagccttctccagtggtgacctctggcggcagagtgacactgcagtgtgagagccaggtggccttcggcggctttatcctgtgcaaggagggcgaggaggagcacccccagtgtctgaatagccagcctcacgcccggggcagctccagagccatcttctctgtgggccctgtgagcccaaaccggagatggtcccacaggtgctacggctatgacctgaacagcccatacgtgtggtctagcccctctgatctgctggagctgctggtgcctggcgtgagcaagaagccatctctgagcgtgcagccaggccctgtggtggcacctggcgagtctctgaccctgcagtgcgtgagcgacgtgggctacgatcggttcgtgctgtataaggagggagagagggatctgaggcagctgccaggcagacagcctcaggcaggactgtcccaggcaaactttacactgggccccgtgagccggagctacggcggacagtaccgctgctatggagcacacaatctgtcctctgagtgttctgcccccagcgaccccctggacatcctgatcaccggccagatcaggggcacaccattcatcagcgtgcagccaggaccaaccgtggcctccggcgagaacgtgacactgctgtgccagagctggcgccagttccacacctttctgctgacaaaggcaggagcagcagacgcacctctgaggctgcgctccatccacgagtacccaaagtatcaggccgagtttccaatgagccccgtgacctccgcccacgcaggcacatacagatgctatggcagcctgaacagcgacccctacctgctgagccacccttccgagccactggagctggtggtgtcc

SEQ ID NO: 98 - protein, 198 aa, Artificial sequence
  LILRB2 D1-2, AA 22-219 (Q8N423-1 UniParc) AA SEQUENCE
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVP

SEQ ID NO: 99 - DNA, 594 bp, Artificial sequence
  LILRB2 D1-2, AA 22-219 (Q8N423-1 UniParc) NA SEQUENCE
cagaccggcacaatccccaagcctaccctgtgggccgagccagatagcgtgatcacccagggctcccccgtgacactgtcttgccagggcagcctggaggcacaggagtaccggctgtatagagagaagaagagcgcctcctggatcacccggatcagacccgagctggtgaagaacggccagtttcacatcccttccatcacctgggagcacacaggccggtacggatgccagtactattctcgggccagatggagcgagctgtccgaccccctggtgctggtcatgaccggcgcctatccaaagcccacactgtccgcccagccttctccagtggtgacctctggcggcagagtgacactgcagtgtgagagccaggtggccttcggcggctttatcctgtgcaaggagggcgaggaggagcacccccagtgtctgaatagccagcctcacgcccggggcagctccagagccatcttctctgtgggccctgtgagcccaaaccggagatggtcccacaggtgctacggctatgacctgaacagcccatacgtgtggtctagcccctctgatctgctggagctgctggtgcct

SEQ ID NO: 100 - protein, 697 aa, Artificial sequence
  Siglec 10 full length (Q96LC7-1 Uniprot) AA SEQUENCE
MLLPLLLSSLLGGSQAMDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYSPKLLGPSCSWEAEGLHCSCSSQASPAPSLRWWLGEELLEGNSSQDSFEVTPSSAGPWANSSLSLHGGLSSGLRLRCEAWNVHGAQSGSILQLPDKKGLISTAFSNGAFLGIGITALLFLCLALIIMKILPKRRTQTETPRPRFSRHSTILDYINVVPTAGPLAQKRNQKATPNSPRTPLPPGAPSPESKKNQKKQYQLPSFPEPKSSTQAPESQESQEELHYATLNFPGVRPRPEARMPKGTQADYAEVKFQ

SEQ ID NO: 101 - protein, 128 aa, Artificial sequence
  Siglec 10, AA 18-145 NO MUTATION (Q96LC7-1 Uniprot) AA SEQUENCE
DGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQK

SEQ ID NO: 102 - DNA, 186 bp, Artificial Sequence
  Siglec 10, AA 18-145 NO MUTATION (Q96LC7-1 Uniprot) NA SEQUENCE
gatggccggttttggatcagagtgcaggagtccgtgatggtgcctgagggcctgtgcatcagcgtgccatgctccttctcttaccccagacaggactggaccggctctacacccgcctacggctattggtttaaggccgtgaccgagacaacaaagggcgcccctgtggccacaaaccaccagagc

SEQ ID NO: 103 - protein, 128 aa, Artificial sequence
  Siglec 10, AA 18-145 plus C36S (Q96LC7-1 Uniprot) AA SEQUENCE
DGRFWIRVQESVMVPEGLSISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQK

SEQ ID NO: 104 - DNA, 384 bp, Artificial sequence
  Siglec 10, AA 18-145 plus C36S (Q96LC7-1 Uniprot) NA SEQUENCE
gatggccggttttggatcagagtgcaggagtccgtgatggtgcctgagggcctgtctatcagcgtgccatgctccttctcttaccccagacaggactggaccggctctacacccgcctacggctattggtttaaggccgtgaccgagacaacaaagggcgcccctgtggccacaaaccaccagagcagagaggtggagatgtccacccggggcagattccagctgacaggcgaccccgccaagggcaattgtagcctggtcatcagggacgcccagatgcaggatgagtctcagtacttctttagggtggagcgcggcagctacgtgcgctataactttatgaatgatggcttctttctgaaggtgaccgccctgacacagaag

SEQ ID NO: 105 - protein, 534 aa, Artificial sequence
  Siglec 10 full ECD, AA 17-550 (Q96LC7-1 Uniprot) AA SEQUENCE
MDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYSPKLLGPSCSWEAEGLHCSCSSQASPAPSLRWWLGEELLEGNSSQDSFEVTPSSAGPWANSSLSLHGGLSSGLRLRCEAWNVHGAQSGSILQLPDKKGLISTAFSN

SEQ ID NO: 106 - protein, 244 aa, Artificial sequence
  TIGIT full length (Q495A1 Uniprot ID) AA SEQUENCE
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 107 - protein, 112 aa, Artificial sequence
  TIGIT, AA 22-134 NO MUTATIONS (Q495A1 Uniprot ID) AA SEQUENCE
MTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEH

SEQ ID NO: 108 - DNA, 336 bp, Artificial sequence
  TIGIT, ECD AA 22-134 NO MUTATIONS (Q495A1 Uniprot ID) NA SEQUENCE
atgaccggcacaatcgagacaacaggcaacatctctgccgagaagggaggcagcatcatcctgcagtgccacctgagcagcaccacagcccaggtgacccaggtgaactgggagcagcaggaccagctgctggccatctgcaatgccgatctgggctggcacatcagcccctcctttaaggatagggtggcacctggaccaggcctgggcctgaccctgcagagcctgaccgtgaatgacacaggcgagtacttctgtatctaccacacatatcctgatggcacctatacaggcagaatctttctggaggtgctggagtctagcgtggccgagcac

SEQ ID NO: 109 - protein, 112 aa, Artificial sequence
  TIGIT, AA 22-134 plus I42A C69S (Q495A1 Uniprot ID) AA SEQUENCE
MTGTIETTGNISAEKGGSIALQCHLSSTTAQVTQVNWEQQDQLLAISNADLGWHISPSFKDRVAPGPGLGLTLQSLTVNDTGEYFCIYHTYPDGTYTGRIFLEVLESSVAEH

SEQ ID NO: 110 - DNA, 336 bp, Artificial sequence
  TIGIT, AA 22-134 plus I42A C69S (Q495A1 Uniprot ID) NA SEQUENCE
atgaccggcacaatcgagacaacaggcaacatctctgccgagaagggaggcagcatcgccctgcagtgccacctgagcagcaccacagcccaggtgacccaggtgaactgggagcagcaggaccagctgctggccatctccaatgccgatctgggctggcacatcagcccctcctttaaggatagggtggcacctggaccaggcctgggcctgaccctgcagagcctgaccgtgaatgacacaggcgagtacttctgtatctaccacacatatcctgatggcacctatacaggcagaatctttctggaggtgctggagtctagcgtggccgagcac

SEQ ID NO: 111 - protein, 112 aa, Artificial sequence
  TIGIT, AA 22-134 C69S (Q495A1 Uniprot ID in DSP 502V2) 112 AA, SEQUENCE
MTGTIETTGNISAEKGGSIILQCHLSSTTAQVTQVNWEQQDQLLAISNADLGWHISPSFKDRVAPGPGLGLTLQSLTVNDTGEYFCIYHTYPDGTYTGRIFLEVLESSVAEH

SEQ ID NO: 112 - DNA, 336 bp, Artificial sequence
  TIGIT, AA 22-134 C69S (Q495A1 Uniprot ID in DSP 502V2)336 NA, SEQUENCE
atgaccggcacaatcgagacaacaggcaacatctctgccgagaagggaggcagcatcatcctgcagtgccacctgagcagcaccacagcccaggtgacccaggtgaactgggagcagcaggaccagctgctggccatctccaatgccgatctgggctggcacatcagcccctcctttaaggatagggtggcacctggaccaggcctgggcctgaccctgcagagcctgaccgtgaatgacacaggcgagtacttctgtatctaccacacatatcctgatggcacctatacaggcagaatctttctggaggtgctggagtctagcgtggccgagcac

SEQ ID NO: 113 - protein, 116 aa, Artificial sequence
  TIGIT, ECD AA 22-137 (Q495A1 Uniprot ID in DSP 502V3) 116 AA, SEQUENCE
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGAR

SEQ ID NO: 114 - DNA, 348 bp, Artificial sequence
  TIGIT, ECD AA 22-137 (Q495A1 Uniprot ID in DSP 502V3) 348 NA, SEQUENCE
atgatgaccggcactattgaaactaccggcaacatctctgccgagaagggcggcagcatcatcctccagtgccacctgagcagcaccacagcccaggtgacacaggtgaactgggagcagcaggaccagctgctggccatctgtaatgccgatctgggctggcacatcagcccttccttcaaggacagggtggcccctggcccaggcctgggcctgaccctccagagcctgaccgtgaatgacacaggcgagtacttctgcatctaccacacatatccagatggcacctatacaggccggatctttctggaggtgctggagtctagcgtggcagagcacggcgccaga

SEQ ID NO: 115 - protein, 120 aa, Artificial sequence
  TIGIT full ECD AA 22-141 (Q495A1 Uniprot ID) 120 AA, SEQUENCE
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 116 - protein, 21 aa, Artificial sequence
  artificial signal peptide
MESPAQLLFLLLLWLPDGVHA

SEQ ID NO: 117 - protein, 15 aa, Artificial sequence
  linker amino acid sequence
GGGGSGGGGSGGGGS

SEQ ID NO: 118 - protein, 20 aa, Artificial sequence
  linker amino acid sequence
GGGGSGGGGSGGGGSGGGGS

SEQ ID NO: 119 - protein, 8 aa, Artificial sequence
  linker amino acid sequence
GGGGGGGG

SEQ ID NO: 120 - protein, 6 aa, Artificial sequence
  linker amino acid sequence
GGGGGG

SEQ ID NO: 121 - protein, 14 aa, Artificial sequence
  linker amino acid sequence
GGGGSGGGGSGGGG

SEQ ID NO: 122 - protein, 9 aa, Artificial sequence
  linker amino acid sequence
GGGGSGGGG

SEQ ID NO: 123 - protein, 20 aa, Artificial sequence
  linker amino acid sequence
  REPEAT (6)..(10): may be absent
  REPEAT (11)..(15): may be absent
  REPEAT (16)..(20): may be absent
GGGGSGGGGSGGGGSGGGGS

SEQ ID NO: 124 - protein, 5 aa, Artificial sequence
  linker amino acid sequence
GGGGS

SEQ ID NO: 125 - protein, 10 aa, Artificial sequence
  linker amino acid sequence
GGGGSGGGGS

SEQ ID NO: 126 - protein, 15 aa, Artificial sequence
  linker amino acid sequence
  REPEAT (6)..(10): may be absent
  REPEAT (11)..(15): may be absent
EAAAKEAAAKEAAAK

SEQ ID NO: 127 - protein, 27 aa, Artificial sequence
  linker amino acid sequence
  REPEAT (12)..(16): may be absent
  REPEAT (17)..(21): may be absent
  REPEAT (22)..(26): may be absent
AEAAAKEAAAKEAAAKEAAAKEAAAKA

SEQ ID NO: 128 - protein, 12 aa, Artificial sequence
  linker amino acid sequence
AEAAAKEAAAKA

SEQ ID NO: 129 - protein, 46 aa, Artificial sequence
  linker amino acid sequence
AEAAAKEAAAKEAAAKEAAAKALEAEAAAKEAAAKEAAAKEAAAKA

SEQ ID NO: 130 - protein, 5 aa, Artificial sequence
  linker amino acid sequence
PAPAP

SEQ ID NO: 131 - protein, 18 aa, Artificial sequence
  linker amino acid sequence
KESGSVSSEQLAQFRSLD

SEQ ID NO: 132 - protein, 14 aa, Artificial sequence
  linker amino acid sequence
EGKSSGSGSESKST

SEQ ID NO: 133 - protein, 12 aa, Artificial sequence
  linker amino acid sequence
GSAGSAAGSGEF

SEQ ID NO: 134 - protein, 229 aa, Artificial sequence
  hIgG4 Fc linker 229 aa
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 135 - protein, 229 aa, Artificial sequence
  hIgG4 Fc linker (knob) 229 aa
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 136 - protein, 229 aa, Artificial sequence
  hIgG4 Fc linker (hole) 229 aa
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 137 - protein, 232 aa, Artificial sequence
  hIgG1 Fc linker, (AA 99-330 fromP01857-1 plus C220S (kabat) substitution 232 amino acids.
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 359 aa, Artificial sequence
  short SIRPa- Fc (IgG1 knob)359 AA, first monomer of DSP216V3
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - DNA, 1077 bp, Artificial sequence
  NA of #138
gaggaggagctgcaggtcatccagcccgataagtctgtgctggtggcagcaggagagaccgccacactgaggtgcaccgccacaagcctgatcccagtgggaccaatccagtggtttaggggagcaggccctggcagagagctgatctacaaccagaaggagggccacttcccaagagtgaccacagtgagcgacctgaccaagcggaacaatatggatttttccatcagaatcggcaatatcacacctgccgacgccggcacctactattgcgtgaagttcaggaagggctccccagacgatgtggagtttaagagcggagcaggcaccgagctgtccgtgcgggcaaagggaggaggaggcagcggaggaggaggctccgagcctaagagctccgacaagacccacacatgcccaccatgtcctgcaccagagctgctgggaggaccttccgtgttcctgtttcctccaaagccaaaggatacactgatgatctccagaacaccagaggtgacctgcgtggtggtggacgtgtctcacgaggaccccgaggtgaagtttaactggtacgtggacggcgtggaggtgcacaatgccaagaccaagccaagggaggagcagtacaactccacatatcgcgtggtgtctgtgctgaccgtgctgcaccaggattggctgaacggcaaggagtataagtgtaaggtgagcaataaggccctgcccgcccctatcgagaagaccatctccaaggcaaagggacagcccagggagcctcaggtgtacacactgcccccttgccgcgacgagctgaccaagaaccaggtgtctctgtggtgtctggtgaagggcttctacccatctgacatcgccgtggagtgggagagcaatggccagcccgagaacaattacaagaccacaccacccgtgctggacagcgatggctccttctttctgtattccaagctgacagtggacaagtctcggtggcagcagggcaacgtgttttcctgttctgtgatgcacgaggccctgcacaatcactatacccagaagagcctgtccctgtctcccggcaag

SEQ ID NO: 140 - protein, 356 aa, Artificial sequence
  short SIRPa- Fc (IgG4 knob)356 AA, first monomer of DSP216V4
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 141 - DNA, 1068 bp, Artificial sequence
  NA of #140
gaggaggagctgcaggtcatccagcccgataagtctgtgctggtggcagcaggagagaccgccacactgaggtgcaccgccacaagcctgatcccagtgggaccaatccagtggtttaggggagcaggccctggcagagagctgatctacaaccagaaggagggccacttcccaagagtgaccacagtgagcgacctgaccaagcggaacaatatggatttttccatcagaatcggcaatatcacacctgccgacgccggcacctactattgcgtgaagttcaggaagggctccccagacgatgtggagtttaagagcggagcaggcaccgagctgtccgtgcgggcaaagggaggaggaggatccggaggaggaggatccgagtctaagtatggaccaccatgccctccatgtccagcacctgagtttgagggaggacctagcgtgttcctgtttccccctaagccaaaggacacactgatgatctccaggacaccagaggtgacctgcgtggtggtggacgtgtctcaggaggatcccgaggtgcagttcaactggtacgtggatggcgtggaggtgcacaatgccaagaccaagcctagggaggagcagtttaactctacataccgcgtggtgagcgtgctgaccgtgctgcaccaggattggctgaacggcaaggagtataagtgtaaggtgagcaataagggcctgccaagctccatcgagaagaccatctccaaggcaaagggacagccaagggagcctcaggtgtgcacactgccaccctctcaggaggagatgaccaagaaccaggtgagcctgtggtgtctggtgaagggcttctacccaagcgacatcgccgtggagtgggagtccaatggccagcccgagaacaattacaagaccacacctccagtgctggactctgatggcagcttctttctgtattctaggctgacagtggataagagccgctggcaggagggcaacgtgtttagctgttccgtgatgcacgaggccctgcacaatcactatacccagaagtctctgagcctgtccctgggcaag

SEQ ID NO: 142 - protein, 585 aa, Artificial sequence
  SIRPa- Fc (IgG1 knob LALA)358 AA, first monomer of DSP216V5
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - DNA, 1755 bp, Artificial sequence
  NA of #142
gaggaggagctgcaggtcatccagcccgataagtctgtgctggtggcagcaggagagaccgccacactgaggtgcaccgccacaagcctgatcccagtgggaccaatccagtggtttaggggagcaggccctggcagagagctgatctacaaccagaaggagggccacttcccaagagtgaccacagtgagcgacctgaccaagcggaacaatatggatttttccatcagaatcggcaatatcacacctgccgacgccggcacctactattgcgtgaagttcaggaagggctccccagacgatgtggagtttaagagcggagcaggcaccgagctgtccgtgcgggcaaagccttccgccccagtggtgtctggaccagcagccagagccaccccacagcacacagtgtccttcacctgtgagtctcacggctttagcccccgggacatcaccctgaagtggttcaagaacggcaatgagctgtctgactttcagaccaacgtggaccccgtgggcgagtctgtgagctattccatccactctacagccaaggtggtgctgacccgcgaggacgtgcacagccaggtcatctgcgaggtggcacacgtgaccctgcagggcgatcctctgaggggcacagccaatctgagcgagaccatcagagtgccccctacactggaggtgacccagcagcccgtgcgcgcagagaaccaagtgaatgtgacatgtcaggtgaggaagttctaccctcagcgcctgcagctgacctggctggagaacggcaacgtgagccggaccgagacagccagcaccgtgacagagaacaaggacggcacatataattggatgtcttggctgctggtgaacgtgagcgcccacagggacgatgtgaagctgacctgccaggtggagcacgacggacagccagccgtgtctaagagccacgatctgaaggtgagcgcccaccctaaggagcagggctccaacacagccgccgagaataccggcagcaacgagcggaatatctacggaggaggaggcagcggaggaggaggctccgagcctaagagctccgacaagacccacacatgcccaccatgtcctgcaccagaggcagcaggaggaccttccgtgttcctgtttcctccaaagccaaaggatacactgatgatctccagaacaccagaggtgacctgcgtggtggtggacgtgtctcacgaggaccccgaggtgaagtttaactggtacgtggacggcgtggaggtgcacaatgccaagaccaagccaagggaggagcagtacaactccacatatcgcgtggtgtctgtgctgaccgtgctgcaccaggattggctgaacggcaaggagtataagtgtaaggtgagcaataaggccctgcccgcccctatcgagaagaccatctccaaggcaaagggacagcccagggagcctcaggtgtacacactgcccccttgccgcgacgagctgaccaagaaccaggtgtctctgtggtgtctggtgaagggcttctacccatctgacatcgccgtggagtgggagagcaatggccagcccgagaacaattacaagaccacaccacccgtgctggacagcgatggctccttctttctgtattccaagctgacagtggacaagtctcggtggcagcagggcaacgtgttttcctgttctgtgatgcacgaggccctgcacaatcactatacccagaagagcctgtccctgtctcccggcaag

SEQ ID NO: 144 - protein, 359 aa, Artificial sequence
  short SIRPa- Fc (IgG1 knob LALA )359 AA, first monomer of DSP216V6
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - DNA, 1077 bp, Artificial sequence
  NA of #144
gaggaggagctgcaggtcatccagcccgataagtctgtgctggtggcagcaggagagaccgccacactgaggtgcaccgccacaagcctgatcccagtgggaccaatccagtggtttaggggagcaggccctggcagagagctgatctacaaccagaaggagggccacttcccaagagtgaccacagtgagcgacctgaccaagcggaacaatatggatttttccatcagaatcggcaatatcacacctgccgacgccggcacctactattgcgtgaagttcaggaagggctccccagacgatgtggagtttaagagcggagcaggcaccgagctgtccgtgcgggcaaagggaggaggaggcagcggaggaggaggctccgagcctaagagctccgacaagacccacacatgcccaccatgtcctgcaccagaggcagcaggaggaccttccgtgttcctgtttcctccaaagccaaaggatacactgatgatctccagaacaccagaggtgacctgcgtggtggtggacgtgtctcacgaggaccccgaggtgaagtttaactggtacgtggacggcgtggaggtgcacaatgccaagaccaagccaagggaggagcagtacaactccacatatcgcgtggtgtctgtgctgaccgtgctgcaccaggattggctgaacggcaaggagtataagtgtaaggtgagcaataaggccctgcccgcccctatcgagaagaccatctccaaggcaaagggacagcccagggagcctcaggtgtacacactgcccccttgccgcgacgagctgaccaagaaccaggtgtctctgtggtgtctggtgaagggcttctacccatctgacatcgccgtggagtgggagagcaatggccagcccgagaacaattacaagaccacaccacccgtgctggacagcgatggctccttctttctgtattccaagctgacagtggacaagtctcggtggcagcagggcaacgtgttttcctgttctgtgatgcacgaggccctgcacaatcactatacccagaagagcctgtccctgtctcccggcaag

SEQ ID NO: 146 - protein, 354 aa, Artificial sequence
  TIGIT- Fc (IgG1 knob LALA )354 AA, first monomer of DSP502V4
MTGTIETTGNISAEKGGSIALQCHLSSTTAQVTQVNWEQQDQLLAISNADLGWHISPSFKDRVAPGPGLGLTLQSLTVNDTGEYFCIYHTYPDGTYTGRIFLEVLESSVAEHGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - DNA, 1062 bp, Artificial sequence
  NA of #146
atgaccggcacaatcgagacaacaggcaacatctctgccgagaagggaggcagcatcgccctgcagtgccacctgagcagcaccacagcccaggtgacccaggtgaactgggagcagcaggaccagctgctggccatctctaatgccgatctgggctggcacatcagcccatcctttaaggatagggtggcaccaggaccaggcctgggcctgaccctgcagagcctgaccgtgaatgacacaggcgagtacttctgtatctaccacacatatcccgatggcacctatacaggcagaatctttctggaggtgctggagtctagcgtggccgagcacggaggaggaggcagcggaggaggaggctccgagcctaagtcctctgacaagacccacacatgccccccttgtcctgcaccagaggcagcaggcggaccttccgtgttcctgtttccacccaagccaaaggataccctgatgatctccaggacccctgaggtgacatgcgtggtggtggacgtgtctcacgaggaccccgaggtgaagttcaactggtacgtggacggcgtggaggtgcacaatgccaagacaaagcctcgggaggagcagtacaactccacctatagagtggtgtctgtgctgacagtgctgcaccaggattggctgaacggcaaggagtataagtgtaaggtgagcaataaggccctgcccgcccctatcgagaaaaccatcagcaaggcaaagggacagccaagggagccacaggtgtacaccctgcctccatgccgcgacgagctgacaaagaaccaggtgagcctgtggtgtctggtgaagggcttctatccatctgacatcgccgtggagtgggagagcaatggccagcccgagaacaattacaagaccacaccccctgtgctggactccgatggctctttctttctgtatagcaagctgaccgtggacaagtccagatggcagcagggcaacgtgttttcttgcagcgtgatgcacgaggccctgcacaatcactacacacagaagtccctgtctctgagccccggcaag

SEQ ID NO: 148 - protein, 382 aa, Artificial sequence
  PD1- Fc (IgG1 hole LALA )382 AA, second monomer of DSP502V4
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 149 - DNA, 1146 bp, Artificial sequence
  NA of #148
gattcacccgatagaccttggaacccacctaccttctcccccgccctgctggtggtgacagagggcgacaatgccaccttcacatgctcttttagcaacacctccgagtctttcgtgctgaattggtacaggatgagcccctccaaccagacagataagctggccgcatttccagaggaccgcagccagccaggacaggattcccggttcagagtgacccagctgcctaatggccgggactttcacatgtctgtggtgagagcccggagaaacgatagcggcacatacctgtgcggagccatctccctggcccctaaggcacagatcaaggagtccctgagggcagagctgagggtgaccgagaggagggcagaggtgccaacagcacacccttctccaagcccccggcctgcaggacagggaggaggaggctccggcggcggcggctctgagccaaagagctccgacaagacccacacatgcccaccatgtccagcaccagaggcagcaggaggacctagcgtgttcctgtttcctccaaagccaaaggataccctgatgatctctaggaccccagaggtgacatgcgtggtggtggacgtgagccacgaggaccccgaggtgaagtttaattggtacgtggacggcgtggaggtgcacaacgccaagacaaagcctagggaggagcagtacaattctacctatcgcgtggtgagcgtgctgacagtgctgcaccaggattggctgaatggcaaggagtataagtgtaaggtgtccaacaaggccctgcctgccccaatcgagaagaccatctctaaggcaaagggacagccccgggagcctcaggtgtgcaccctgccccctagcagagacgagctgacaaagaatcaggtgtccctgtcttgtgccgtgaagggcttctaccccagcgacatcgcagtggagtgggagtccaacggacagcctgagaacaattataagaccacaccacccgtgctggactctgatggcagcttctttctggtgtccaagctgaccgtggacaagtctcggtggcagcagggcaacgtgtttagctgctccgtgatgcacgaagcactgcacaaccactacacccagaagtcactgtcactgtccccaggaaag

SEQ ID NO: 150 - protein, 645 aa, Artificial sequence
  LILRB2- Fc (IgG1 hole LALA )382 AA, second monomer of DSP216V5 and  DSP216V6
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 151 - DNA, 2019 bp, Artificial sequence
  NA of #150
tctagagccaccatggagtccccagcacagctgctgttcctgctgctgctgtggctgcctgacggagtgcacgcacagaccggcacaatcccaaagcccaccctgtgggccgagcctgattccgtgatcacccagggctctccagtgacactgtcctgccagggctctctggaggcccaggagtaccggctgtatagagagaagaagtctgccagctggatcacccggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagcggaggaggaggctccggaggaggaggctctggcggcggcggcagcgagcctaagagctccgacaagacccacacatgcccaccttgtccagcacctgaggcagcaggaggaccatccgtgttcctgtttccacccaagcctaaggataccctgatgatctctcgcacccctgaggtgacatgcgtggtggtggacgtgagccacgaggaccccgaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtacaagtgtaaggtgtccaataaggccctgccagcccccatcgagaagaccatctctaaggcaaagggacagcccagggagcctcaggtgtgcaccctgcctccaagccgcgacgagctgacaaagaaccaggtgtctctgagctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctaggtggcagcagggcaacgtgttttcctgttctgtgatgcacgaggccctgcacaatcactacacacagaagagcctgtccctgtctcccggcaagtgagatatc

SEQ ID NO: 152 - protein, 232 aa, Artificial sequence
  IgG1 Fc Hole LALA, Fc (AA 99-330) C220S L234A, L235A plus x93Holex94 T366S and L368A Y407V) and Y349C (the numbers from KABAT) used in DSP216V5, DSP216V6 and DSP502V4
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 153 - DNA, 696 bp, Artificial sequence
  NA of #152
gagcctaagagctccgacaagacccacacatgcccaccttgtccagcacctgaggcagcaggaggaccatccgtgttcctgtttccacccaagcctaaggataccctgatgatctctcgcacccctgaggtgacatgcgtggtggtggacgtgagccacgaggaccccgaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtacaagtgtaaggtgtccaataaggccctgccagcccccatcgagaagaccatctctaaggcaaagggacagcccagggagcctcaggtgtgcaccctgcctccaagccgcgacgagctgacaaagaaccaggtgtctctgagctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctaggtggcagcagggcaacgtgttttcctgttctgtgatgcacgaggccctgcacaatcactacacacagaagagcctgtccctgtctcccggcaag

SEQ ID NO: 154 - protein, 232 aa, Artificial sequence
  IgG1 Fc x93knobx94 LALA (Fc IgG1 (AA 99-330) P01857-1 C220S (kabat) plus x93Knobx94 T366W (kabat) and S354C (kabat) plus L234A, L235A (kabat), used in DSP216v5 and DSP502V4
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - DNA, 698 bp, Artificial sequence
  NA of #154
  misc_feature (1)..(1): n is a, c, g, or t
nagagcctaagagctccgacaagacccacacatgcccaccatgtcctgcaccagaggcagcaggaggaccttccgtgttcctgtttcctccaaagccaaaggatacactgatgatctccagaacaccagaggtgacctgcgtggtggtggacgtgtctcacgaggaccccgaggtgaagtttaactggtacgtggacggcgtggaggtgcacaatgccaagaccaagccaagggaggagcagtacaactccacatatcgcgtggtgtctgtgctgaccgtgctgcaccaggattggctgaacggcaaggagtataagtgtaaggtgagcaataaggccctgcccgcccctatcgagaagaccatctccaaggcaaagggacagcccagggagcctcaggtgtacacactgcccccttgccgcgacgagctgaccaagaaccaggtgtctctgtggtgtctggtgaagggcttctacccatctgacatcgccgtggagtgggagagcaatggccagcccgagaacaattacaagaccacaccacccgtgctggacagcgatggctccttctttctgtattccaagctgacagtggacaagtctcggtggcagcagggcaacgtgttttcctgttctgtgatgcacgaggccctgcacaatcactatacccagaagagcctgtccctgtctcccggcaag

SEQ ID NO: 156 - protein, 232 aa, Artificial sequence
  hIgG1 Fc
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 229 aa, Artificial sequence
  hIgG4-SPLE- Fc knob
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 158 - protein, 229 aa, Artificial sequence
  hIgG4 Fc knob
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 159 - protein, 229 aa, Artificial sequence
  hIgG4 Fc hole
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 160 - protein, 232 aa, Artificial sequence
  hIgG1 Fc knob only
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 232 aa, Artificial sequence
  hIgG1 Fc hole only
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 162 - protein, 232 aa, Artificial sequence
  hIgG1 C220S Fc Hole
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 229 aa, Artificial sequence; identical to SEQ ID NO: 157
  hIgG4-SPLE- Fc knob NO Y349C

SEQ ID NO: 164 - protein, 229 aa, Artificial sequence
  hIgG4-SPLE- Fc Hole NO E356C
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK